(19) United States
(12) Patent Application Publication
(10) Pub. No.: US 2009/0054427 A1
(43) Pub. Date: Feb. 26, 2009

(54) AMINOBENZYL-SUBSTITUTED CYCLIC SULFONES USEFUL AS BACE INHIBITORS

(76) Inventors: Emmanuelle Briard, Huningue (FR); Rainer Martin Lueoend, Therwil (CH); Rainer Machauer, Freiburg (DE); Henrik Moebitz, Freiburg (DE); Olivier Rogel, Hesingue (FR); Jean-Michel Rondeau, Rixheim (FR); Heinrich Rueeger, Flueh (CH); Marina Tintelnot-Blomley, Maulburg (DE); Siem Jacob Veenstra, Lorrach (DE)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/196,461

(22) Filed: Aug. 22, 2008

(30) Foreign Application Priority Data
Aug. 23, 2007 (EP) 07114881.1
Aug. 23, 2007 (GB) 07114881.1
Jul. 10, 2008 (EP) 08160123.9
Jul. 10, 2008 (GB) 08160123.9

(51) Int. Cl. A61K 31/5377; C07D 335/02; A61K 31/382; A61P 25/00; C07D 413/08

(52) U.S. Cl. 514/231.5; 549/28; 514/432; 544/145

(57) ABSTRACT

The invention relates to novel heterocyclic compounds of the formula

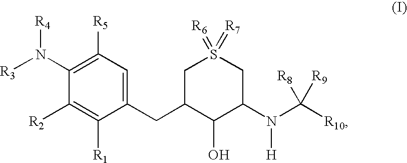

in which all of the variables are as defined in the specification, in free form or in salt form, to their preparation, to their use as medicaments and to medicaments comprising them.

The present invention relates to novel heterocyclic compounds, to their preparation, to their use as medicaments and to medicaments comprising them.

More particularly, the invention relates to a compound of the formula

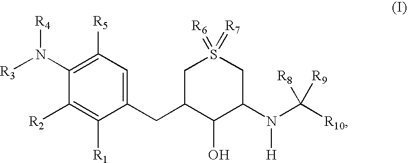

in which
R₁ is hydrogen; halogen; or (C₁₋₈)alkyl;
R₂ is hydrogen; halogen; (C₁₋₈)alkyl; halogen-(C₁₋₈)alkyl; (C₁₋₈)alkoxy; or halogen-(C₁₋₈)alkoxy;
either
R₃ is hydrogen; and
R₄ is hydrogen; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
or
R₃ is halogen-(C₁₋₈)alkyl; hydroxy-(C₁₋₈)alkyl; (C₁₋₈)alkoxy-(C₁₋₈)alkyl; formyl; (C₁₋₈)-alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or an aryl-(C₁₋₈)alkyl group, which aryl-(C₁₋₈)alkyl group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)-alkoxy; and
R₄ is hydrogen; (C₁₋₈)alkyl; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;

R₅ is hydrogen; halogen; (C₁₋₈)alkyl; halogen-(C₁₋₈)alkyl; (C₂₋₈)alkenyl; (C₃₋₈)cycloalkyl-(C₂₋₈)alkenyl; halogen-(C₂₋₈)alkenyl; (C₁₋₈)alkoxy; halogen-(C₁₋₈)alkoxy; (C₁₋₈)alkoxy-(C₁₋₈)alkyl; halogen-(C₁₋₈)alkoxy-(C₁₋₈)alkyl; (C₁₋₈)alkoxy-(C₁₋₈)alkoxy-(C₁₋₈)alkyl; halogen-(C₁₋₈)alkoxy-(C₁₋₈)alkoxy-(C₁₋₈)alkyl; formyl; (C₁₋₈)alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or a (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group, which (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)alkoxy;
either
R₆ is absent; and
R₇ is absent;
or
R₆ is oxo; and
R₇ is absent;
or
R₆ is oxo; and
R₇ is oxo; imino; (C₁₋₈)alkylimino; benzylimino; formylimino; or (C₁₋₈)alkylcarbonyl-imino;
either
R₈ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl; and
R₉ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl;
or
R₈ and R₉, taken together, complete, together with the carbon atom, to which they are attached, a (C₃₋₈)cycloalkylidene moiety, in which (C₃₋₈)cycloalkylidene moiety 1 of its —CH₂— ring members can be replaced with —O—; and
R₁₀ is an aryl or heteroaryl group, which aryl or heteroaryl group is optionally mono-, di-, tri- or tetra-substituted by substituents independently selected from the group, consisting of halogen, hydroxy, (C₁₋₈)alkyl, halogen-(C₁₋₈)alkyl, hydroxy-(C₁₋₈)alkyl, hydroxy-(C₁₋₈)alkyl substituted by halogen, (C₁₋₈)alkoxy-(C₁₋₈)alkyl, halogen-(C₁₋₈)alkoxy-(C₁₋₈)alkyl, cyano-(C₁₋₈)alkyl, (C₁₋₈)alkoxy, halogen-(C₁₋₈)alkoxy, a heteroaryl group, which heteroaryl group is optionally substituted by 1 to 4 substituents independently selected from the group, consisting of halogen, (C₁₋₈)alkyl and halogen-(C₁₋₈)alkyl, and a (C₃₋₈)cycloalkyl group, in which (C₃₋₈)cycloalkyl group 1 of its —CH₂— ring members can be replaced with —O—, and which (C₃₋₈)cycloalkyl group, in which 1 of its —CH₂— ring members is optionally replaced with —O—, is optionally substituted by 1 to 4 substituents independently selected from the group, consisting of halogen, (C₁₋₈)alkyl and halogen-(C₁₋₈)alkyl, in free form or in salt form.

E.g. on account of one or more than one asymmetrical carbon atom, which may be present in a compound of the formula I, a corresponding compound of the formula I may exist in pure optically active form or in the form of a mixture of optical isomers, e.g. in the form of a race-mic mixture. All of such pure optical isomers and all of their mixtures, including the racemic mixtures, are part of the present invention.

A compound of the formula I may exist in free form or in salt form, e.g. a basic compound in acid addition salt form or an acidic compound in the form of a salt with a base. All of such free compounds and salts are part of the present invention.

A compound of the formula I may exist in tautomeric form. All of such tautomers are part of the present invention.

The present invention includes all pharmaceutically acceptable isotope-labeled compounds of the formula I, wherein one or more than one atom is/are replaced by one or more than one atom having the same atomic number as, but an atomic mass different from, the one(s) usually found in nature. Examples of such isotopes are those of carbon, such as ¹¹C, ¹³C or ¹⁴C, chlorine, such as ³⁶Cl, fluorine, such as ¹⁸F, bromine, such as ⁷⁶Br, hydrogen, such as ²H or ³H, iodine, such as ¹²³I, ¹²⁴I, ¹²⁵I or ¹³¹I, nitrogen, such as ¹³N or ¹⁵N, oxygen, such as ¹⁵O, ¹⁷O or ¹³O, phosphorus, such as ³²P, or sulphur, such as ³⁵S. An isotope-labeled compound of the formula I can be prepared by a process analogous to those described in the Examples or by a conventional technique known to those skilled in the art using an appropriate isotopically-labeled reagent or starting material. The incorporation of a heavier isotope, such as ²H, may provide greater metabolic stability to a compound of the formula I, which may result in, for example, an increased in vivo-half-life of the compound or in reduced dosage requirements. Certain isotope-labeled compounds of the formula I, for example those incorporating a radioactive isotope, such as ³H or ¹⁴C, may be used in drug or substrate-tissue distribution studies. Compounds of the formula I with a positron emitting isotope, such as ¹¹C, ¹⁸F, ¹³N or ¹⁵O, may be useful in positron emission tomography (PET) or single photon emission computed tomography (SPECT) studies, e.g. to examine substrate-receptor occupancies.

Halogen denotes fluorine, chlorine, bromine or iodine.

A halogenated group or moiety, such as halogenalkyl, can be mono-, poly- or per-halo-genated.

An aryl group, ring or moiety is a naphthyl or, preferably, phenyl group, ring or moiety.

A heteroaryl group, ring or moiety is an aromatic 5- or 6-membered structure, in which structure 1, 2, 3 or 4 ring members are hetero ring members independently selected from the group, consisting of a nitrogen ring member, an oxygen ring member and a sulfur ring member, such as furyl, pyrrolyl, thienyl, pyrazolyl, imidazolyl, thiazolyl, isothiazolyl, oxazolyl, isoxazolyl, triazolyl, tetrazolyl, pyridazinyl, pyrimidyl or pyridyl.

A non-aromatic heterocyclyl group, ring or moiety is a non-aromatic 4-, 5-, 6- or 7-membered cyclic structure, in which cyclic structure 1, 2 or 3 ring members are hetero ring members independently selected from the group, consisting of a nitrogen ring member, an oxygen ring member and a sulfur ring member, such as oxetanyl, pyrrolinyl, pyrrolidyl, tetrahydrofuryl, tetrahydrothienyl, piperidyl, piperazinyl, tetrahydropyranyl, morpholinyl or perhydroazepinyl.

Any non-cyclic carbon containing group or moiety with more than 1 carbon atom is straight-chain or branched.

Unless defined otherwise, carbon containing groups, moieties or molecules contain 1 to 8, preferably 1 to 6, preferably 1 to 4, preferably 1 or 2, carbon atoms.

In preferred embodiments, the invention relates to a compound of the formula I, in free form or in salt form, in which

(1) R₁ is hydrogen; halogen; or (C₁₋₈)alkyl; preferably hydrogen;

(2) R₂ is hydrogen; halogen; (C₁₋₈)alkyl; halogen-(C₁₋₈)alkyl; (C₁₋₈)alkoxy; or halogen-(C₁₋₈)-alkoxy;

preferably hydrogen; halogen; (C₁₋₈)alkoxy; or halogen-(C₁₋₈)alkoxy;

preferably hydrogen; halogen; (C₁₆)alkoxy; or halogen-(C_{1 6})alkoxy;

(3) either
R₃ is hydrogen; and
R₄ is hydrogen; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
or
R₃ is halogen-(C₁₋₈)alkyl; hydroxy-(C₁₋₈)alkyl; (C₁₋₈)alkoxy-(C₁₋₈)alkyl; formyl; (C₁₋₈)-alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or an aryl-(C₁₋₈)alkyl group, which aryl-(C₁₋₈)alkyl group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)-alkoxy; and
R₄ is hydrogen; (C₁₋₈)alkyl; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;

preferably R₃ is hydrogen; and R₄ is hydrogen;

(4) R₅ is hydrogen; halogen; (C₁₋₈)alkyl; halogen-(C₁₋₈)alkyl; (C₂₋₈)alkenyl; (C₃₋₈)cycloalkyl-(C₂₋₈)alkenyl; halogen-(C₂₋₈)alkenyl; (C₁₋₈)alkoxy; halogen-(C₁₋₈)alkoxy; (C₁₋₈)alkoxy-(C₁₋₈)alkyl; halogen-(C₁₋₈)alkoxy-(C₁₋₈)alkyl; (C₁₋₈)alkoxy-(C₁₋₈)alkoxy-(C₁₋₈)alkyl; halogen-(C₁₋₈)alkoxy-(C₁₋₈)alkoxy-(C₁₋₈)alkyl; formyl; (C₁₋₈)alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxy-carbonyl; or a (C₃₋₈)cycloalkyl, (C₃₋₈)cycloalkyl-(C₁₋₈)alkyl, (C₃₋₈)cycloalkyl-(C₁₋₈)alkoxy, (C₃₋₈)-cycloalkoxy, aryl, aryloxy, heteroaryl, heteroaryloxy, non-aromatic heterocyclyl or non-aromatic heterocyclyloxy group, which (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)-alkoxy and halogen-(C₁₋₈)alkoxy;

preferably hydrogen; halogen; (C₁₋₈)alkyl; halogen-(C₁₋₈)alkyl; (C₂₋₈)alkenyl; formyl; (C₁₋₈)-alkylcarbonyl; or a heteroaryl group, which heteroaryl group is optionally substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)alkoxy;

preferably hydrogen; halogen; (C₁₋₈)alkyl; halogen-(C₁₋₈)alkyl; (C₂₋₈)alkenyl; (C₁₋₈)alkylcarbonyl; or heteroaryl;

preferably hydrogen; halogen; (C₁₋₈)alkyl; halogen-(C₁₋₁₆)alkyl; (C₂₋₈)alkenyl; (C₁₆)alkylcarbonyl; or furyl;

(5) either
R₆ is absent; and
R₇ is absent;
or
R₆ is oxo; and
R₇ is absent;
or
R₆ is oxo; and
R₇ is oxo; imino; (C₁₋₈)alkylimino; benzylimino; formylimino; or (C₁₋₈)alkylcarbonylimino;

preferably either
R₆ is absent; and
R₇ is absent;
or
R₆ is oxo; and
R₇ is absent;
or
R₆ is oxo; and
R₇ is oxo; or imino;

preferably either
R₆ is oxo; and
R₇ is absent;
or
R₆ is oxo; and
R₇ is oxo; or imino;

preferably either
R₆ is oxo; and
R₇ is absent;
or
R₆ is oxo; and
R₇ is oxo;

preferably R₆ is oxo; and R₇ is oxo;

(6) either
R₈ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl; and
R₉ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl;
or
R₈ and R₉, taken together, complete, together with the carbon atom, to which they are attached, a (C₃₋₈)cycloalkylidene moiety, in which (C₃₋₈)cycloalkylidene moiety 1 of its —CH₂— ring members can be replaced with —O—;

preferably either
R₈ is hydrogen; or (C₁₋₈)alkyl; and
R₉ is hydrogen;
or
R₈ and R₉, taken together, complete, together with the carbon atom, to which they are attached, a (C₃₋₈)cycloalkylidene moiety;

preferably either
R₈ is hydrogen; or (C₁₋₈)alkyl; and
R₉ is hydrogen;
or
R₈ and R₉, taken together, complete, together with the carbon atom, to which they are attached, a cyclopropylidene moiety;

preferably R₈ is hydrogen; or (C₁₋₆)alkyl; and R₉ is hydrogen;

preferably R₈ is hydrogen; and R₉ is hydrogen;

(7) R₁₀ is an aryl or heteroaryl group, which aryl or heteroaryl group is optionally substituted by 1 to 4 substituents independently selected from the group, consisting of halogen, hydroxy, (C₁₋₈)alkyl, halogen-(C₁₋₈)alkyl, hydroxy-(C₁₋₈)alkyl, halogen-substituted hydroxy-(C₁₋₈)alkyl, (C₁₋₈)alkoxy-(C₁₋₈)alkyl, halogen-(C₁₋₈)alkoxy-(C₁₋₈)alkyl, cyano-(C₁₋₈)alkyl, (C₁₋₈)alkoxy, halogen-(C₁₋₈)alkoxy, a heteroaryl group, which heteroaryl group is optionally substituted by 1 to 4 substituents independently selected from the group, consisting of halogen, (C₁₋₈)alkyl and halogen-(C₁₋₈)alkyl, and a (C₃₋₈)cycloalkyl group, in which (C₃₋₈)cycloalkyl group 1 of its —CH₂— ring members can be replaced with —O—, and which (C₃₋₈)cycloalkyl group, in which 1 of its —CH₂— ring members is optionally replaced with —O—, is optionally substituted by 1 to 4 substituents independently selected from the group, consisting of halogen, (C₁₋₈)alkyl and halogen-(C₁₋₈)alkyl;

preferably an aryl or heteroaryl group, which aryl or heteroaryl group is optionally substituted by 1 to 4 substituents independently selected from the group, consisting of halogen, hydroxy, (C₁₋₈)alkyl, halogen-(C₁₋₈)alkyl, hydroxy-(C₁₋₈)alkyl, a heteroaryl group, which heteroaryl group is optionally substituted by 1 to 4 substituents independently selected from the group, consisting of halogen, (C₁₋₈)alkyl and halogen-(C₁₋₈)alkyl, and a (C₃₋₈)cycloalkyl group, in which (C₃₋₈)cycloalkyl group 1 of its —CH₂— ring members can be replaced with —O—, and which (C₃₋₈)-cycloalkyl group, in which 1 of its —CH₂— ring members is optionally replaced with —O—, is optionally substituted by 1 to 4 substituents independently selected from the group, consisting of halogen, (C₁₋₈)alkyl and halogen-(C₁₋₈)alkyl;

preferably an aryl or heteroaryl group, which aryl or heteroaryl group is optionally substituted by 1 or 2 substituents independently selected from the group, consisting of halogen, hydroxy, (C₁₋₈)alkyl, halogen-(C₁₋₈)alkyl, hydroxy-(C₁₋₈)alkyl, an unsubstituted heteroaryl group and an oxetanyl group, which oxetanyl group is optionally substituted by 1 or 2 substituents independently selected from the group, consisting of halogen, (C₁₋₈)alkyl and halogen-(C₁₋₈)-alkyl;

preferably a phenyl, isoxazolyl or pyrazolyl group, which phenyl, isoxazolyl or pyrazolyl group is optionally substituted by 1 or 2 substituents independently selected from the group, consisting of halogen, hydroxy, (C₁₋₈)alkyl, halogen-(C₁₋₈)alkyl, hydroxy-(C₁₋₈)alkyl, an unsubstituted pyrazolyl group and an oxetanyl group, which oxetanyl group is optionally substituted by 1 or 2 substituents independently selected from the group, consisting of (C₁₋₈)alkyl;

preferably a phenyl, isoxazolyl or pyrazolyl group, which phenyl, isoxazolyl or pyrazolyl group is substituted by 1 or 2 substituents independently selected from the group, consisting of halogen, hydroxy, (C₁₋₈)alkyl, halogen-(C₁₋₆)alkyl, hydroxy-(C₁₋₆)alkyl, an unsubstituted pyrazolyl group and an oxetanyl group, which oxetanyl group is substituted by 1 or 2 substituents independently selected from the group, consisting of (C₁₋₈)alkyl;

(8) R₁ is hydrogen; halogen; or (C₁₋₈)alkyl;
R₂ is hydrogen; halogen; (C₁₋₈)alkyl; halogen-(C₁₋₈)alkyl; (C₁₋₈)alkoxy; or halogen-(C₁₋₈)alkoxy;
either
R₃ is hydrogen; and
R₄ is hydrogen; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
or
R₃ is halogen-(C₁₋₈)alkyl; hydroxy-(C₁₋₈)alkyl; (C₁₋₈)alkoxy-(C₁₋₈)alkyl; formyl; (C₁₋₈)-alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or an aryl-(C₁₋₈)alkyl group, which aryl-(C₁₋₈)alkyl group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)-alkoxy; and
R₄ is hydrogen; (C₁₋₈)alkyl; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
R₅ is hydrogen; halogen; (C₁₋₈)alkyl; halogen-(C₁₋₈)alkyl; (C₂₋₈)alkenyl; (C₃₋₈)cycloalkyl-(C₂₋₈)alkenyl; halogen-(C₂₋₈)alkenyl; (C₁₋₈)alkoxy; halogen-(C₁₋₈)alkoxy; (C₁₋₈)alkoxy-(C₁₋₈)alkyl; halogen-(C₁₋₈)alkoxy-(C₁₋₈)alkyl; (C₁₋₈)alkoxy-(C₁₋₈)alkoxy-(C₁₋₈)alkyl; halogen-(C₁₋₈)alkoxy-(C₁₋₈)alkoxy-(C₁₋₈)alkyl; formyl; (C₁₋₈)alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or a (C₃₋₈)cycloalkyl, (C₃₋₈)cycloalkyl-(C₁₋₈)alkyl, (C₃₋₈)cycloalkyl-(C₁₋₈)alkoxy, (C₃₋₈)-cycloalkoxy, aryl, aryloxy, heteroaryl, heteroaryloxy, non-aromatic heterocyclyl or non-aromatic heterocyclyloxy group, which (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)-alkoxy and halogen-(C₁₋₈)alkoxy;
either
R₆ is absent; and
R₇ is absent;
or
R₆ is oxo; and
R₇ is absent;
or
R₆ is oxo; and
R₇ is oxo; imino; (C₁₋₈)alkylimino; benzylimino; formylimino; or (C₁₋₈)alkylcarbonylimino;
either
R₈ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl; and
R₉ is hydrogen; (C₁₋₈)alkyl; halogen-(C₁₋₈)alkyl; hydroxy-(C₁₋₈)alkyl; (C₁₋₈)alkoxy-(C₁₋₈)-alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl;
or
R₈ and R₉, taken together, complete, together with the carbon atom, to which they are attached, a (C₃₋₈)cycloalkylidene moiety, in which (C₃₋₈)cycloalkylidene moiety 1 of its —CH₂— ring members can be replaced with —O—; and

R₁₀ is an aryl or heteroaryl group, which aryl or heteroaryl group is optionally substituted by 1 to 4 substituents independently selected from the group, consisting of halogen, hydroxy, (C₁₋₈)alkyl, halogen-(C₁₋₈)alkyl, hydroxy-(C₁₋₈)alkyl, halogen-substituted hydroxy-(C₁₋₈)alkyl, (C₁₋₁₈)alkoxy-(C₁₋₈)alkyl, halogen-(C₁₋₈)alkoxy-(C₁₋₈)alkyl, cyano-(C₁₋₈)alkyl, (C₁₋₈)alkoxy, halogen-(C₁₋₈)alkoxy, a heteroaryl group, which heteroaryl group is optionally substituted by 1 to 4 substituents independently selected from the group, consisting of halogen, (C₁₋₈)alkyl and halogen-(C₁₋₈)alkyl, and a (C₃₋₈)cycloalkyl group, in which (C₃₋₈)cycloalkyl group 1 of its —CH₂— ring members can be replaced with —O—, and which (C₃₋₈)cycloalkyl group, in which 1 of its —CH₂— ring members is optionally replaced with —O—, is optionally substituted by 1 to 4 substituents independently selected from the group, consisting of halogen, (C₁₋₈)alkyl and halogen-(C₁₋₈)alkyl.

The preferred embodiments (1) to (8) are preferred independently, collectively or in any combination or sub-combination.

In especially preferred embodiments, the invention relates to one or more than one of the compounds of the formula I mentioned in the Examples hereinafter, in free form or in salt form.

In a further aspect, the invention relates to a process for the preparation of a compound of the formula I, in free form or in salt form, comprising the steps of

a) for the preparation of a compound of the formula I, in free form or in salt form, in which R₃ is hydrogen and R₄ is hydrogen, treatment of a compound of the formula

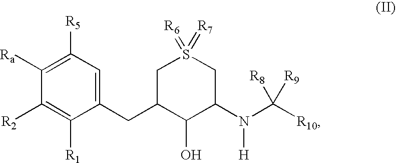

in which Rₐ is azido or nitro and all of the other variables are as defined for the formula I, in free form or in salt form, with a reducing agent, in order to convert Rₐ into amino, or

b) for the preparation of a compound of the formula I, in free form or in salt form, in which R₈ is hydrogen, treatment of a compound of the formula

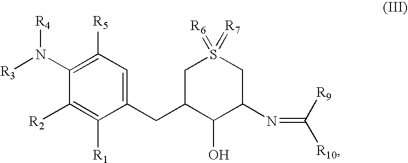

in which all of the variables are as defined for the formula I, in free form or in salt form, with a reducing agent, in order to convert the moiety —N═C(R₉)R₁₀ into the moiety —N(H)—C(H)(R₉)R₁₀,

in each case optionally followed by reduction, oxidation or other functionalisation of the resulting compound and/or by cleavage of any protecting group(s) optionally present,

and of recovering the so obtainable compound of the formula I in free form or in salt form.

The reactions can be effected according to conventional methods, for example as described in the Examples.

The working-up of the reaction mixtures and the purification of the compounds thus obtainable may be carried out in accordance with known procedures.

Salts may be prepared from free compounds in known manner, and vice-versa.

Compounds of the formula I can also be prepared by further conventional processes, which processes are further aspects of the invention, e.g. as described in the Examples.

The starting materials of the formulae II and III are known or may be prepared according to conventional procedures starting from known compounds, for example as described in the Examples.

Compounds of the formula I, in free form or in pharmaceutically acceptable salt form, hereinafter often referred to as “agents of the invention”, exhibit valuable pharmacological properties, when tested in vitro or in vivo, and are, therefore, useful in medicaments.

E.g., agents of the invention are inhibitors of aspartic proteases and can be used for the treatment of a condition, disease or disorder involving processing by such enzymes. Particularly, agents of the invention inhibit beta-secretase and, thus, the generation of beta-amyloid and the subsequent aggregation into oligomers and fibrils.

The inhibiting properties of an agent of the invention towards proteases can be evaluated, e.g., in a test as described hereinafter.

Test 1: Inhibition of Human BACE

Recombinant BACE (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic fluorescence-quenched peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-activity as a function of the test compound concentration.

Test 2: Inhibition of Human BACE-2

Recombinant BACE-2 (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic peptide substrate, derived from the sequence of APP and containing a suitable fluorophore-quencher pair, is added to a final concentration of 1 to 5 μM, and the increase in fluorescence is recorded at a suitable excitation/emission wave-length in a microplate spectro-fluorimeter for 5 to 30 minutes in 1-minute intervals. IC₅₀ values are calculated from percentage of inhibition of BACE-2-activity as a function of the test compound concentration.

Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in sodium formate or sodium acetate buffer at a suitable pH within the range of pH 3.0 to 5.0. Synthetic peptide substrate Mca-Gly-Lys-Pro-Ile-Leu-Phe-Phe-Arg-Leu-Lys(DNP)-D-Arg-NH₂ is added to a final concentration of 1 to 5 μM, and the increase in fluorescence is recorded at excitation of 325 nm and emission at 400 nm in a microplate spectro-fluorimeter for 5 to 30 minutes in 1-minute intervals. IC₅₀ values are calculated from the percentage of inhibition of cathepsin D-activity as a function of the test compound concentration.

Test 4: Inhibition of Cellular Release of Amyloid Peptide 1-40

Chinese hamster ovary cells are transfected with the gene for amyloid precursor protein. The cells are plated at a density of 8000 cells/well into 96-well microtiter plates and cultivated for 24 hours in DMEM cell culture medium containing 10% FCS. The test compound is added to the cells at various concentrations, and the cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-described tests, agents of the invention show activity at concentrations below 50 μM.

Specifically, the agent of the invention described in Example 17 shows an IC₅₀ value of 0.82 μM in Test 4.

Due to their inhibiting properties towards proteases, agents of the invention are useful, e.g., in the treatment or prevention of a neurological or vascular condition, disease or disorder, in which beta-amyloid generation or aggregation plays a role, such as a neurodegenerative condition, disease or disorder, e.g. Alzheimer's disease, Down's syndrome, memory impairment, cognitive impairment, dementia, amyloid neuropathies, brain inflammation, nerve trauma, brain trauma, vascular amyloidosis or cerebral haemorrhage with amyloidosis, or, based on the inhibition of BACE-2 (beta-site APP-cleaving enzyme 2) or cathepsin D, which are close homologues of the pepsin-type aspartyl proteases and beta-secretase, and the correlation of the BACE-2 or cathepsin D expression with a more tumorigenic or metastatic potential of tumor cells, in the suppression of the metastasis process associated with tumor cells.

For the above-mentioned indications, the appropriate dosage will vary depending on, e.g., the compound employed as active pharmaceutical ingredient, the host, the mode of administration, the nature and severity of the condition, disease or disorder or the effect desired. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 0.5 to about 2000, preferably from about 2 to about 200, mg of an agent of the invention conveniently administered, for example, in divided doses up to four times a day or in sustained release form.

An agent of the invention may be administered by any conventional route, in particular enterally, preferably orally, e.g. in the form of a tablet or capsule, or parenterally, e.g. in the form of an injectable solution or suspension.

In accordance with the foregoing, in a further aspect, the invention relates to an agent of the invention for use as a medicament, e.g. for the treatment or prevention of a neurological or vascular condition, disease or disorder, in which beta-amyloid generation or aggregation plays a role, or for the suppression of the metastasis process associated with tumor cells.

In a further aspect, the invention relates to the use of an agent of the invention as active pharmaceutical ingredient in a medicament, e.g. for the treatment or prevention of a neurological or vascular condition, disease or disorder, in which beta-amyloid generation or aggregation plays a role, or for the suppression of the metastasis process associated with tumor cells.

In a further aspect, the invention relates to a pharmaceutical composition comprising an agent of the invention as active pharmaceutical ingredient in association with at least one pharmaceutically acceptable carrier or diluent. Such a composition may be manufactured in conventional manner, e.g. by mixing its components. Unit dosage forms contain, e.g., from about 0.1 to about 1000, preferably from about 1 to about 500, mg of an agent of the invention.

An agent of the invention can be administered as sole active pharmaceutical ingredient or as a combination with at least one other active pharmaceutical ingredient effective, e.g., in the treatment or prevention of a neurological or vascular condition, disease or disorder, in which beta-amyloid generation or aggregation plays a role, or in the suppression of the metastasis process associated with tumor cells. Such a pharmaceutical combination may be in the form of a unit dosage form, which unit dosage form comprises a predetermined quantity of each of the at least two active components in association with at least one pharmaceutically acceptable carrier or diluent. Alternatively, the pharmaceutical combination may be in the form of a package comprising the at least two active components separately, e.g. a pack or dispenser-device adapted for the concomitant or separate administration of the at least two active components, in which these active components are separately arranged. In a further aspect, the invention relates to such pharmaceutical combinations.

In a further aspect, the invention relates to the use of an agent of the invention for the manufacture of a medicament for the treatment or prevention of a neurological or vascular condition, disease or disorder, in which beta-amyloid generation or aggregation plays a role, or for the suppression of the metastasis process associated with tumor cells.

In a further aspect, the invention relates to a method for the treatment or prevention of a neurological or vascular condition, disease or disorder, in which beta-amyloid generation or aggregation plays a role, or for the suppression of the metastasis process associated with tumor cells, in a subject in need of such treatment, prevention or suppression, which method comprises administering to such subject an effective amount of an agent of the invention.

The following Examples illustrate the invention, but do not limit it.

### EXAMPLES

Abbreviations

ACN acetonitrile

AcOH acetic acid

BMIBr₃ 1-butyl-3-methylimidazolium tribromide

(Boc)₂O di-tert-butyl dicarbonate

DBU 1,8-diazabicyclo[5.4.0]undec-7-ene

Dess Martin

reagent 1,1,1-triacetoxy-1,1-dihydro-1,2-benziodoxol-3(1H)-one

DIBAL diisobutylaluminium hydride

DIPEA diisopropylethylamine

DMAP N,N-4-dimethylaminopyridine

DME 1,2-dimethoxyethane

DMSO dimethylsulfoxide

EDC 1-(3-dimethylaminopropyl)-3-ethylcarbodiimide hydrochloride

ETA ethanol-ammonia (conc.) 95:5

Et₂O diethylether

EtOAc ethyl acetate

EtOH ethanol

HOAt 1-hydroxy-7-azabenzotriazole

HOBT hydroxy-benzotriazole

ⁱPrOH iso-propanol

MeOH methanol

NaHMDS sodium hexamethyldisilazane

NaOAc sodium acetate

NEt₃ triethylamine

Oxone potassium monopersulfate

Pd₂(dba)₃ tris(dibenzylideneacetone)dipalladium

Pd(PPh₃)₄ tetrakis(triphenylphosphine)palladium(O)

Ph₅FcP(tBu)₂ 1,2,3,4,5-Pentaphenyl-1′-(di-tert-butylphosphino)ferrocene

PPh₃ triphenylphosphine

P^{t}Bu₃ tri-tert-butylphosphine

p-TsOH para-toluenesulfonic acid

^{t}BuOMe tert-butyl-methyl-ether

TEMPO 2,2,6,6-tetramethylpiperidine 1-oxyl

TFA trifluoroacetic acid

THF tetrahydrofuran

General HPLC Information Concerning Examples 1 to 22

HPLC method A (Rt_{A}):

HPLC-column dimensions: 50×5 mm

HPLC-column type: Nucleosil® 5C₁₋₈, 3 microns

HPLC-eluent: A) water+0.1 Vol.-% TFA
B) ACN+0.1 Vol.-% TFA

HPLC-gradient: 10-100% B in 3 min+1 min 100%B flow=4 ml/min

HPLC method B (Rt_{B}):

HPLC-column dimensions: 125×4 mm

HPLC-column type: MN Nucleodur C18 Pyramid 5 microns

HPLC-eluent: A) water+0.1 Vol.-% TFA
B) ACN+0.1 Vol.-% TFA

HPLC-gradient: 5% A to 100% B in 20 min flow=1 ml/min

HPLC method C (Rt_{C}):

HPLC-column dimensions: 2.1×50 mm

HPLC-column type: SunFire C₁₈, 5 microns

HPLC-eluent: A) ACN
B) water+0.1 Vol.-% TFA

HPLC-gradient: 20-95% A in 3.5 min+95% A in 0.5 min+95-20% A in 0.5 min flow=0.8 ml/min

HPLC method D (Rt_{D}):

HPLC-column dimensions: 5×100 mm

HPLC-column type: Machery-Nagel LiChrospher RP-18

HPLC-eluent: A) ACN
B) water+0.1 Vol.-% TFA

HPLC-gradient: 10-100% A in 5 min flow=1.5 ml/min

HPLC method E (Rt_{E}):

HPLC-column dimensions: 4.6×100 mm

HPLC-column type: XTerra MS C₁₋₈, 3.5 mm

HPLC-eluent: A) water+0.1 Vol.-% TFA
B) ACN+0.1 Vol.-% TFA

HPLC-gradient: 5% B for 1 min, 5-10% B in 4 min, 50-100% B in 2 min flow=0.9 ml/min

### Example 1

### 3S*,4S*,5R*)-3-(4-Amino-3-fluoro-benzyl)-5-(3-tert-butyl-benzylamino)-1,1-dioxo-hexahydro-1lambda*6*-thiopyran-4-ol hydrochloride

### a) 4-tert-Butoxycarbonylamino-3-oxo-5-tritylsulfanyl-pentanoic acid allyl ester

To a solution of di-imidazol-1-yl-methanone (13.9 g, 84 mmol) in anhydrous THF (300 mL) is added dropwise a solution of (R*)-2-tert-butoxycarbonylamino-3-tritylsulfanyl-propionic acid (32.8 g, 70 mmol) and DMAP (0.26 g, 2.1 mmol) in THF (200 mL) at 25° C. over a period of 1 h. The reaction mixture is stirred for 2 h at ambient temperature before a solution of propanedioic acid mono-2-propenyl ester magnesium complex (13.6 g, 42 mmol) dissolved in THF (200 mL) is added. The reaction mixture is stirred for 16 h at 40-45° C. and then evaporated. The residue is re-dissolved in EtOAc and washed with cold 10% citric acid, water, saturated NaHCO₃ solution and brine, dried over MgSO₄ and evaporated. The title compound is obtained after flash-chromatograpy on silica gel (hexane-EtOAc 10:1 to 4:1) as a white crystalline solid after crystallization from Et₂O-hexane: TLC (hexane-EtOAc 1:1) Rf=0.64; HPLC Rt_{A}=2.64 min; ESIMS [M−H]⁺=544; ¹H-NMR (400 MHz, CDCl₃): δ 7.2-7.5 (m, 15H), 5.84 (m, 1H), 5.16 (m, 2H), 5.02 (d, 1H), 4.57 (m, 2H), 4.08 (m, 1H), 3.36 (m, 2H), 2.72 (dd, 1H), 2.54 (dd, 1H), 1.42 (s, 9H).

### b) 5-tert-Butoxycarbonylamino-4-oxo-tetrahydro-thiopyran-3-carboxylic acid allyl ester

To a solution of 4-tert-butoxycarbonylamino-3-oxo-5-tritylsulfanyl-pentanoic acid allyl ester (24.0 g, 44 mmol) in AcOH (200 mL) is added piperidine (5.3 g, 61.6 mmol) and paraform-aldehyde (1.46 g, 46 mmol) and the reaction mixture is stirred at 80° C. for 0.5 h. The reaction mixture is concentrated under reduced pressure and the residual solid is dissolved in EtOAc and washed with saturated NaHCO₃ solution and brine, dried over MgSO₄ and evaporated. The title compound is obtained after flash-chromatograpy on silica gel (toluene-EtOAc 40:1 to 10:1) and crystallization from diisopropyl ether as a white solid: TLC (toluene-EtOAc 10:1) Rf=0.32; HPLC Rt_{A}=1.91 min; ESIMS [M−H]⁺=314; ¹H-NMR (400 MHz, CDCl₃): δ 5.94 (m, 1H), 5.68 (m, 1H), 5.35 (d, 1H), 5.27 (d, 1H), 4.62 (m, 3H), 3.87 (dd, 1H), 3.38 (dd, 1H), 3.22 (t, 1H), 3.04 (dt, 1H), 2.68 (m, 1H), 1.42 (s, 9H).

### c) 5-tert-Butoxycarbonylamino-3-(3-fluoro-4-nitro-benzyl)-4-oxo-tetrahydro-thiopyran-3-carboxylic acid allyl ester

To a solution of 5-tert-butoxycarbonylamino-4-oxo-tetrahydro-thiopyran-3-carboxylic acid allyl ester (2.55 g, 8.08 mmol) in acetone (150 mL) is added K₂CO₃ (3.38 g, 24.2 mmol) and 4-bromomethyl-2-fluoro-1-nitro-benzene (2.12 g, 8.9 mmol) and the reaction mixture is stirred at 25° C. for 16 h. After dilution with water the product is extracted with EtOAc. Combined extracts are washed with brine, dried over MgSO₄ and evaporated. The product obtained as a light yellow solid is suitable for use in the next step: TLC (hexane-EtOAc 1:3) Rf=0.26; HPLC Rt_{A}=2.31 min; ESIMS [M+H+NH₃]⁺=486.

### d) [(3R*,5S*)-5-(3-Fluoro-4-nitro-benzyl)-4-oxo-tetrahydro-thiopyran-3-yl]-carbamic acid tert-butyl ester

To a degassed solution of 5-tert-butoxycarbonylamino-3-(3-fluoro-4-nitro-benzyl)-4-oxo-tetra-hydro-thiopyran-3-carboxylic acid allyl ester (3.56 g, 7.6 mmol) and morpholine (1.4 mL, 15.2 mmol) in THF (50 mL) is added under argon Pd(PPh₃)₄ (0.092 g, 0.076 mmol), and the mixture is stirred at 25° C. for 3 h. The mixture is poured onto cold saturated NaHCO₃ solution and extracted with EtOAc. Combined extracts are washed with brine, dried over MgSO₄ and evaporated. The residue is re-dissolved in THF (10 mL) and kept at 25° C. for 3 h after addition of a catalytic amount of DBU. The title compound is obtained after evaporation and crystallization from diisopropylether as single diastereoisomer: TLC (hexane-EtOAc 1:2) Rf=0.38; HPLC Rt_{A}=2.17 min; ESIMS [M+H+NH₃]⁺=402; ¹H-NMR (400 MHz, CDCl₃): δ 8.0 (dd, 1H), 7.14 (m, 2H), 5.68 (m, 1H), 4.54 (m, 1H), 3.41 (m, 1H), 3.28 (dd, 1H), 3.14 (m, 1H), 2.84 (ddd, 1H), 2.69 (dd, 1H), 2.65 (m, 2H), 1.42 (s, 9H).

### e) [(3R*,4S*,5S*)-5-(3-Fluoro-4-nitro-benzyl)-4-hydroxy-tetrahydro-thiopyran-3-yl]-carbamic acid tert-butyl ester

To a solution of calcium borohydride bis-THF complex (1.14 g, 4.6 mmol) in anhydrous THF (100 mL) is added under argon a solution of [(3R*,5S*)-5-(3-fluoro-4-nitro-benzyl)-4-oxo-tetrahydro-thiopyran-3-yl]-carbamic acid tert-butyl ester (1.77 g, 4.6 mmol) in THF (50 mL) at −70° C. The mixture is slowly warmed to −40° C. and stirred for 1 h at −40° C. The mixture is poured onto a cold aqueous KHSO₄ solution and the product is extracted with EtOAc. The combined extracts are washed with NaHCO₃ solution and brine, dried over MgSO₄ and evaporated. The mixture of diastereoisomers is separated by flash-chromatograpy on silica gel (toluene-EtOAc 6:1 to 3:1) to yield the undesired [(3R*,4R*,5S*)-diastereoisomer and the desired [(3R*,4S*,5S*)-diastereoisomer after crystallization from EtOAc-hexane as a white crystalline solid: TLC (toluene-EtOAc 3:1) Rf=0.20; HPLC Rt_{A}=1.94 min; ESIMS [M+H−56, isobutylene)]⁺=331; ¹H-NMR (600 MHz, DMSO-d₆): δ 8.09 (t, 1H), 7.41 (d, 1H), 7.27 (d, 1H), 6.67 (d, 1H), 4.94 (d, 1H), 3.42 (m, 1H), 3.27 (dd, 1H), 2.91 (m, 1H), 2.55 (m, 2H), 2.39 (dd, 1H), 2.24 (m, 2H), 1.93 (m, 1H), 1.37 (s, 9H).

### f) [(3R*,4S*,5S*)-5-(3-Fluoro-4-nitro-benzyl)-4-hydroxy-1,1-dioxo-hexahydro-1lambda*6*-thiopyran-3-yl]-carbamic acid tert-butyl ester

To a solution of [(3R*,4S*,5S*)-5-(3-fluoro-4-nitro-benzyl)-4-hydroxy-tetrahydro-thiopyran-3-yl]-carbamic acid tert-butyl ester (0.78 g, 2.0 mmol) in THF (15 mL) is added water (15 mL) and oxone (2.62 g, 4.2 mmol), and the mixture is stirred for 2 h at 25° C. The excess oxone is destroyed after addition of 2 equivalents of NaOAc with sodium meta-bisulfite and the product is extracted with EtOAc. The combined extracts are washed with brine, dried over MgSO₄ and evaporated. The product obtained as a light yellow solid is suitable for use in the next step: TLC (toluene-EtOAc 1:1) Rf=0.18; HPLC Rt_{A}=1.67 min; ESIMS [M+H+NH₃]⁺=436; ¹H-NMR (600 MHz, DMSO-d₆): δ 8.11 (t, 1H), 7.41 (d, 1H), 7.27 (d, 1H), 6.71 (d, 1H), 5.22 (d, 1H), 3.72 (m, 1H), 3.0-3.2 (m, 5H), 2.82 (s, 1H), 2.71 (dd, 1H), 2.17 (dd, 1H), 1.38 (s, 9H).

### g) (3R*,4S*,5S*)-3-Amino-5-(3-fluoro-4-nitro-benzyl)-1,1-dioxo-hexahydro-1lambda*6*-thiopyran-4-ol hydrochloride

[(3R*,4S*,5S*)-5-(3-Fluoro-4-nitro-benzyl)-4-hydroxy-1,1-dioxo-hexahydro-1lambda*6*-thio-pyran-3-yl]-carbamic acid tert-butyl ester (0.82 g, 1.94 mmol) in 4N HCl in dioxane (10 mL) is stirred for 1 h at 25° C. and 0.5 h at 40° C. The mixture is evaporated and the product recrystallized from MeOH-Et₂O to yield the title compound as light yellow crystals: TLC (CH₂Cl₂-MeOH—AcOH—H₂O 180:20:2:1) Rf=0.19; ESIMS [M+H]⁺=319; ¹H-NMR (400 MHz, CD₃OD): δ 8.07 (t, 1H), 7.34 (d, 1H), 7.26 (d, 1H), 3.3-3.8 (m, 5H), 3.20 (dd, 1H), 2.91 (dt, 1H), 2.68 (dd, 1H), 2.42 (m, 1H).

### h) (3R*,4S*,5S*)-3-(3-tert-Butyl-benzylamino)-5-(3-fluoro-4-nitro-benzyl)-1,1-dioxo-hexahydro-1lambda*6*-thiopyran-4-ol

To a solution of (3R*,4S*,5S*)-3-amino-5-(3-fluoro-4-nitro-benzyl)-1,1-dioxo-hexahydro-1lambda*6*-thiopyran-4-ol hydrochloride (0.14 g, 0.39 mmol) in MeOH—CH₂Cl₂ 1:1 (3 mL) is added NaOAc (0.065 g, 0.78 mmol) and 3-tert-butyl-benzaldehyde (0.07 g, 0.43 mmol). The reaction mixture is stirred at 25° C. for 0.5 h before NaBH₃CN (0.039 g, 0.59 mmol) is added followed by stirring for 16 h. The mixture is acidified with 1N HCl, stirred for 15 min, basified with K₂CO₃-solution and extracted with EtOAc. The combined organic layers are washed with brine, dried over MgSO₄ and evaporated. The title compound is obtained after purifycation by flash-chromatography on silica gel (hexane-CH2Cl₂—MeOH 20:20:1 to 1:20:5) as a light yellow foam: TLC (EtOAc) Rf=0.45; HPLC Rt_{A}=1.78 min; ESIMS [M+H]⁺=465; ¹H-NMR (400 MHz, CDCl₃): δ 8.02 (t, 1H), 7.1-7.4 (m, 6H), 4.18 (s, 1H), 3.88 (d, 1H), 3.78 (d, 1H), 3.64 (m, 1H), 3.41 (ddd, 1H), 3.24 (ddd, 1H), 3.12 (dt, 1H), 3.00 (dd, 1H), 2,84, (m, 2H), 2.69 (m, 2H), 2.44 (m, 1H), 1.33 (s, 9H).

### i) (3S*,4S*,5R*)-3-(4-Amino-3-fluoro-benzyl)-5-(3-tert-butyl-benzylamino)-1,1-dioxo-hexahydro-1lambda*6*-thiopyran-4-ol hydrochloride

To a solution of (3R*,4S*,5S*)-3-(3-tert-butyl-benzylamino)-5-(3-fluoro-4-nitro-benzyl)-1,1-di-oxo-hexahydro-1lambda*6*-thiopyran-4-ol (0.16 g, 0.34 mmol) in MeOH (10 mL) is added NiCl₂-6H₂O (0.059 g, 0.34 mmol) and at 0-5° C. NaBH₄ (0.054 g, 1.36 mmol) in small portions over a period of 15 min. After stirring for 20 min at 0-5° C. the reaction is quenched by addition of H₂O (0.5 mL). The mixture is filtered through a plug of Celite and evaporated. The residues is taken up in EtOAc and washed NaHCO₃ solution and brine, dried over MgSO₄ and evaporated. The free base is transferred into the hydrochloride salt with 1 N HCl in Et₂O and pure product is obtained after crystallization from ACN-diisopropylether as a light beige solid: TLC (EtOAc) Rf=0.52; HPLC Rt_{A}=1.43 min; ESIMS [M+H]⁺=435; ¹H-NMR (400 MHz, CDCl₃): δ 7.2-7.4 (m, 3H), 7.14 (d, 1H), 6.82 (d, 1H), 6.71 (m, 2H), 4.05 (s, 1H), 3.90 (d, 1H), 3.6-3.8 (m, 3H), 3.38 (ddd, 1H), 3.1 (m, 2H), 2.91 (dt, 1H), 2.5-2,8, (m, 3H), 2.34 (m, 1H), 1.34 (s, 9H).

### Example 2

### 3S,4S,5R)-3-(4-Amino-3-bromo-5-fluoro-benzyl)-5-(3-tert-butyl-benzyl-amino)-1,1-dioxo-hexahydro-1lambda*6*-thiopyran-4-ol hydrochloride

### a) (3R*,5S*)-3-Amino-5-(3-fluoro-4-nitro-benzyl)-tetrahydro-thiopyran-4-ol hydro-chloride

[(3R*,5S*)-5-(3-Fluoro-4-nitro-benzyl)-4-hydroxy-tetrahydro-thiopyran-3-yl]-carbamic acid tert-butyl ester (1.0 g, 2.56 mmol) dissolved in 4N HCl in dioxane (15 mL) is stirred for 1 h at 25° C. The reaction mixture is evaporated and the product recrystallized from MeOH-Et₂O to yield the title compound as a mixture of diastereoisomers as white crystals: TLC (CH₂Cl₂-MeOH—AcOH—H₂O 180:20:2:1) Rf=0.22 and 0.18; HPLC Rt_{A}=1.20 and 1.24 min; ESIMS [M+H]⁺=287.

### b) (3R*,5S*)-3-(3-tert-Butyl-benzylamino)-5-(3-fluoro-4-nitro-benzyl)-tetrahydro-thio-pyran-4-ol

To a solution of (3R*,5S*)-3-amino-5-(3-fluoro-4-nitro-benzyl)-tetrahydro-thiopyran-4-ol hydrochloride (0.83 g, 2.55 mmol) in MeOH—CH₂Cl₂ 1:1 (40 mL) is added NaOAc (0.63 g, 7.6 mmol) and 3-tert-butyl-benzaldehyde (0.48 g, 2.67 mmol). The mixture is stirred at 25° C. for 0.5 h before NaBH₃CN (0.039 g, 0.59 mmol) is added followed by stirring for 16 h. The mixture is acidified with 1N HCl, stirred for 15 min, basified with K₂CO₃-solution and extracted with EtOAc. The combined organic layers are washed with brine, dried over MgSO₄ and evaporated. The title compound obtained as a light yellow solid is suitable for use in the next step: TLC (EtOAc) Rf=0.45 and 0.30; HPLC Rt_{A}=1.92 min; ESIMS [M+H]⁺=433.

### c) (3-tert-Butyl-benzyl)-[(3R*,4R*,5S*)-5-(3-fluoro-4-nitro-benzyl)-4-hydroxy-tetrahydro-thiopyran-3-yl]-carbamic acid tert-butyl ester

To a solution of (3R*,5S*)-3-(3-tert-butyl-benzylamino)-5-(3-fluoro-4-nitro-benzyl)-tetrahydro-thiopyran-4-ol (1.05 g, 2.4 mmol) in ACN (25 mL) is added (Boc)₂O (0.86 g, 3.85 mmol) and NEt₃ (0.61 mL, 4.33 mmol), and the mixture is heated at 55-60° C. for 20 h and at 70° C. for 1h. The mixture is evaporated and the two diastereoisomers are separated by flash-chromatograpy on silica gel (hexane-EtOAc 6:1 to 1:1) to give the [(3R*,4S*,5S*)-diastere-oisomer: TLC (hexane-EtOAc 3:1) Rf=0.43; HPLC Rt_{A}=2.80 min; ESIMS [M+H-56, isobutylene)]⁺=477 and the [(3R*,4R*,5S*)-diastereoisomer as a light yellow solid: TLC (hexane-EtOAc 3:1) Rf=0.23; HPLC Rt_{A}=2.71 min; ESIMS [M+H-56, isobutylene)]⁺=477.

### d) (3-tert-Butyl-benzyl)-[(3R*,4R*,5S*)-5-(3-fluoro-4-nitro-benzyl)-4-hydroxy-1,1-dioxo-hexahydro-1lambda*6*-thiopyran-3-yl]-carbamic acid tert-butyl ester

To a solution of (3-tert-butyl-benzyl)-[(3R*,4R*,5S*)-5-(3-fluoro-4-nitro-benzyl)-4-hydroxy-tetrahydro-thiopyran-3-yl]-carbamic acid tert-butyl ester (0.85 g, 1.58 mmol) in THF-H₂O (30 mL) is added oxone (2.075 g, 3.32 mmol) and the mixture is stirred at 25° C. for 2 h. Excess oxone is destroyed with Na₂S₂O₅ and the mixture is extracted with EtOAc. The combined organic layers are washed with brine and dried over MgSO₄. After evaporation the title compound is obtained as a light yellow foam suitable for use in the next step: TLC (hexane/EtOAc 1:1) Rf=0.56; HPLC Rt_{A}=2.52 min;ESIMS [M+H+NH₃]⁺=582.

### e) (3-tert-Butyl-benzyl)-[(3R*,5S*)-5-(3-fluoro-4-nitro-benzyl)-1,1,4-trioxo-hexahydro-1lambda*6*-thiopyran-3-yl]-carbamic acid tert-butyl ester

To a solution of (3-tert-Butyl-benzyl)-[(3R*,4R*,5S*)-5-(3-fluoro-4-nitro-benzyl)-4-hydroxy-1,1-dioxo-hexahydro-1lambda*6*-thiopyran-3-yl]-carbamic acid tert-butyl ester (0.891 g, 1.58 mmol) in CH₂Cl₂ (20 mL) is added Dess-Martin reagent (0.898 g, 2.05 mmol) and the mixture is stirred at 25° C. for 2 h. To the mixture is added saturated NaHCO₃-solution and NaS₂O₃-solution and after stirring for 1 h the product is extracted with CH₂Cl₂. Combined extracts are washed with water, dried over MgSO₄, and evaporated. The title compound is obtained as a light yellow oil suitable for use in the next step: TLC (hexane-EtOAc 1:1) Rf=0.59; HPLC Rt_{A}=2.71 min; ESIMS [M+H-56 (isobutylene)]⁺=507.

### f) (3-tert-Butyl-benzyl)-[(3R*,4S*,5S*)-5-(3-fluoro-4-nitro-benzyl)-4-hydroxy-1,1-dioxo-hexahydro-1lambda*6*-thiopyran-3-yl]-carbamic acid tert-butyl ester

To a solution of (3-tert-butyl-benzyl)-[(3R*,5S*)-5-(3-fluoro-4-nitro-benzyl)-1,1,4-trioxo-hexa-hydro-1lambda*6*-thiopyran-3-yl]-carbamic acid tert-butyl ester (0.70 g, 1.23 mmol) in anhydrous THF (30 mL) is added under argon at −60° C. the calcium borohydride bis-THF complex (0.323 g, 1.48 mmol) and the mixture is slowly warmed to −40° C. After stirring at −40° C. for 0.5 h the mixture is quenched by addition of a NaH₂PO₄ solution and the product is extracted with EtOAc. Combined extracts are washed with brine, dried over MgSO₄, and evaporated. The title compound is obtained after flash-chromatograpy on silica gel (hexane-EtOAc 3:1 to 1:1) as a colorless foam: TLC (hexane-EtOAc 1:1) Rf=0.43; HPLC Rt_{A}=2.37 min; [M+H+NH₃]⁺=582.

### g) [(3R*,4S*,5S*)-5-(4-Amino-3-fluoro-benzyl)-4-hydroxy-1,1-dioxo-hexahydro-1lambda*6*-thiopyran-3-yl]-(3-tert-butyl-benzyl)-carbamic acid tert-butyl ester

To a solution of (3-tert-butyl-benzyl)-[(3R*,4S*,5S*)-5-(3-fluoro-4-nitro-benzyl)-4-hydroxy-1,1-dioxo-hexahydro-1lambda*6*-thiopyran-3-yl]-carbamic acid tert-butyl ester (1.0 g, 1.75 mmol) in MeOH (20 mL) is added NiCl₂-6H₂O (0.424 g, 1.75 mmol) and at 0-5° C. NaBH₄ (0.275 g, 7.0 mmol) in small portions over a period of 15 min. After stirring for 20 min at 0-5° C. the mixture is quenched by slow addition of H₂O (5 mL). After removal of the solvent the residue is taken up in EtOAc and filtered through a plug of Celite. The filtrate is washed with NaHCO₃ solution and brine, dried over MgSO₄ and evaporated. The pure title compound is obtained as a white foam suitable for use in the next step: TLC (hexane-EtOAc 1:1) Rf=0.38; HPLC Rt_{A}=2.08 min; ESIMS [M+H-100]⁺=435.

### h) [(3R,4S,5S)-5-(4-Amino-3-bromo-5-fluoro-benzyl)-4-hydroxy-1,1-dioxo-hexahydro-1lambda*6*-thiopyran-3-yl]-(3-tert-butyl-benzyl)-carbamic acid tert-butyl ester

To a solution of [(3R*,4S*,5S*)-5-(4-amino-3-fluoro-benzyl)-4-hydroxy-1,1-dioxo-hexahydro-1lambda*6*-thiopyran-3-yl]-(3-tert-butyl-benzyl)-carbamic acid tert-butyl ester (0.60 g, 1.11 mmol) in CH₂Cl₂ is added under argon at −10° C. a 0.2N solution of BMIBr₃ in CH₂Cl₂(5.6 mL, 1.12 mmol) within 20 min. After stirring for 0.5 h at −10° C. the mixture is quenched with a small amount of a NaS₂O₃ solution. The mixture is diluted with CH₂Cl₂ and washed with NaHCO₃ solution and water, dried over MgSO₄ and evaporated. The title compound is obtained after purification by flash-chromatography on silica gel (hexane-EtOAc 6:1 to 3:1) as a yellow oil: TLC (hexane-EtOAc 3:1) Rf=0.17; HPLC Rt_{A}=2.39 min; ESIMS [M+H-100]⁺=513, 515. The racemate is separated by preparative HPLC on Chiracel OD with heptane-EtOH 95:5 to yield the (3R,4S,5S)-enantiomer (peak 1) with >99% ee as a yellow oil and the (3S,4R,5R)-enantiomer (peak 2) with >98% ee as a white solid.

### i) (3S,4S,5R)-3-(4-Amino-3-bromo-5-fluoro-benzyl)-5-(3-tert-butyl-benzylamino)-1,1-dioxo-hexahydro-1lambda*6*-thiopyran-4-ol hydrochloride

A solution of [(3R,4S,5S)-5-(4-amino-3-bromo-5-fluoro-benzyl)-4-hydroxy-1,1-dioxo-hexa-hydro-1lambda*6*-thiopyran-3-yl]-(3-tert-butyl-benzyl)-carbamic acid tert-butyl ester (0.13 g, 0.21 mmol) in 4N HCl in dioxane (2 mL) is stirred for 0.5 h at 25° C. The mixture is evaporated and the product recrystallized from MeOH-Et₂O to yield the title compound as white crystals: TLC (CH₂Cl₂-MeOH—AcOH—H₂O 180:20:2:1) Rf=0.76; HPLC Rt_{A}=1.79 min; ESIMS [M+H]⁺=513, 515; ¹H-NMR (600 MHz, DMSO-d₆): δ 9.95 (s, 1H), 9.05 (s, 1H), 7.67 (s, 1H), 7.44 (dt, 1H), 7.38 (m, 2H), 7.09 (s, 1H), 6.94 (dd, 1H), 6.1 (s, 1H), 4.25 (m, 2H), 4.15 (s, 2H), 3.85 (dt, 1H), 3.65 (m, 2H), 3.21 (m, 2H), 2.95 (dd, 1H), 2.84 (ddd, 1H), 2.41 (dd, 1H), 2.03 (m, 1H), 1.32 (s, 9H).

### Examples 2a-2b

The compounds listed in Table 1 can be prepared by a procedure analogous to that used in example 2.

TABLE 1

| | | HPLC | |
|---|---|---|---|
| | | Rt [min] | |
| Example | Compound | Method | ESIMS |
| 2a | (3S,4S,5R)-3-(4-Amino-3-bromo-5-fluoro-benzyl)-5- | 6.05 D | [M − H]⁺ = 521/ |
| | [3-(1,1-difluoro-ethyl)-benzylamino]-1,1-dioxo-hexa- | | 523 |
| | hydro-1lambda*6*-thiopyran-4-ol | | |
| 2b | (3S,4S,5R)-3-(4-Amino-3-bromo-5-fluoro-benzyl)-5- | 13.99 B | [M − H]⁺ = 527/ |
| | [3-(2,2-dimethyl-propyl)-benzylamino]-1,1-dioxo-hexahydro- | | 529 |
| | 1lambda*6*-thiopyran-4-ol | | |

### 3-(1,1-Difluoro-ethyl)-benzaldehyde (Example 2a)

### a) 3-(1,1-Difluoro-ethyl)-benzonitrile

3-Acetyl benzonitrile (2.9 g, 20 mmol) and deoxo-fluor (5.52 mL, 30 mmol) is stirred overnight at 85° C. The mixture is basified with saturated NaHCO₃ and is extracted with CH₂Cl₂ (3×30 ml). The combined organic phases are washed with brine, dried over Na₂SO₄, filtered and evaporated. The title compound is obtained after distillation (bp_{0.85} 61° C.) as a colorless syrup: ESIMS [M-CN]⁺=143; ¹⁹F-NMR (376 MHz, CDCl₃): δ −88 (s, 2F); ¹H-NMR (400 MHz, CDCl₃): δ 7.80 (s, 1H), 7.73 (t, 1H), 7.56 (t, 1H), 1.93 (t, 3H).

### b) 3-(1,1-Difluoro-ethyl)-benzaldehyde

A cooled solution of 3-(1,1-difluoro-ethyl)-benzonitrile (0.5 g, 2.99 mmol) in CH₂Cl₂ (10 mL) is treated dropwise with a 1M solution of DIBAL (3.59 mL, 3.59 mmol) at 0° C. The reaction mixture is stirred for 1 h at 0° C. The reaction mixture is poured into a mixture of ice/6N HCl and is stirred for 1 h. The layers are separated and the aqueous phase is extracted with CH₂Cl₂. The organic phases are joined, washed with brine, dried over Na₂SO₄, filtered and evaporated. The title compound is obtained after flash-chromatography on silica gel (cyclohexane-EtOAc 4:1) as a light yellow syrup: ESIMS [2M+H₂O]⁻=358; ¹⁹F-NMR (376 MHz, CDCl₃): δ 88 (s, 2F); ¹H-NMR (400 MHz, CDCl₃): δ 10.06 (s, 1H), 8.03 (s, 1H), 7.95 (d, 1H), 7.77 (d, 1H), 7.62 (t, 1H), 1.96 (t, 3H).

### 3-(2,2-Dimethyl-propyl)-benzaldehyde (Example 2b)

3-(2,2-Dimethyl-propyl)-benzaldehyde ethylene acetal (prepared from 3-bromobenzaldehyde ethylene acetal and neo-pentylmagnesium chloride) (0.661 g, 3 mmol) is stirred in THF (6.6 mL) and sulfuric acid 2M (3 mL) at room temperature for 3.5 h. The mixture is diluted with EtOAc and washed with saturated NaHCO₃ solution and brine, dried over Na₂SO₄ and evaporated. The crude product is filtered through silica gel (hexane/EtOAc 9:1) yielding a slightly yellowish oil: TLC (hexane/EtOAc 9:1) Rf=0.44; ESIMS [M+H+NH₃]⁺=194.

### Example 3

### (3S*,4S*,5R*)-3-(4-Amino-3-bromo-benzyl)-5-(3-tert-butyl-benzylamino)-1,1-dioxo-hexahydro-1lambda*6*-thiopyran-4-ol hydrochloride

The title compound is prepared in analogous manner as described for example 1c)-1i), starting from 5-tert-butoxycarbonylamino-4-oxo-tetrahydro-thiopyran-3-carboxylic acid allyl ester and 3-bromo-4-nitro-benzyl bromide: HPLC Rt_{A}=1.68 min; ESIMS [M+H]⁺=494, 496.

### Example 4

### (3S*,4S*,5R*)-3-[4-Amino-3-(2,2,2-trifluoro-ethoxy)-benzyl]-5-(3-tert-butyl-benzylamino)-1,1-dioxo-hexahydro-1lambda*6*-thiopyran-4-ol hydrochloride

### a) (3R*,4S*,5S*)-3-(3-tert-Butyl-benzylamino)-5-[4-nitro-3-(2,2,2-trifluoro-ethoxy)-benzyl]-1,1-dioxo-hexahydro-1lambda*6*-thiopyran-4-ol

A mixture of (3R*,4S*,5S*)-3-(3-tert-butyl-benzylamino)-5-(3-fluoro-4-nitro-benzyl)-1,1-dioxo-hexahydro-1lambda*6*-thiopyran-4-ol (example 1h)) (0.025 g, 0.05 mmol), 2,2,2-trifluoroethanol (0.107 g, 1.06 mmol) and K₂CO₃ (0.037 g, 0.265 mmol) in DMF (0.5 mL) is heated at 80° C. for 16 h. The mixture is diluted with EtOAc and washed with KHSO₄ solution and brine, dried over MgSO₄ and evaporated. The title compound is obtained as a yellow foam suitable for use in the next step: TLC (EtOAc) Rf=0.35; HPLC Rt_{A}=1.94 min; ESIMS [M+H]⁺=545.

### b) (3S*,4S*,5R*)-3-[4-Amino-3-(2,2,2-trifluoro-ethoxy)-benzyl]-5-(3-tert-butyl-benzyl-amino)-1,1-dioxo-hexahydro-1lambda*6*-thiopyran-4-ol hydrochloride

The title compound is prepared in analogous manner as described for example 2g) starting from (3R*,4S*,5S*)-3-(3-tert-butyl-benzylamino)-5-[4-nitro-3-(2,2,2-trifluoro-ethoxy)-benzyl]-1,1-dioxo-hexahydro-1lambda*6*-thiopyran-4-ol. The purified free base is converted into the HCl salt with 1N HCl in Et₂O: TLC (EtOAc) Rf=0.40; HPLC Rt_{A}=1.50 min; ESIMS [M+H]⁺=515.

### Example 5

### (3S,4S,5R)-3-(4-Amino-3-propoxy-benzyl)-5-(3-tert-butyl-benzylamino)-1,1-dioxo-hexahydro-1lambda*6*-thiopyran-4-ol hydrochloride

### a) (3-tert-Butyl-benzyl)-[(3R*,4S*,5S*)-4-hydroxy-5-(4-nitro-3-propoxy-benzyl)-1,1-dioxo-hexahydro-1lambda*6*-thiopyran-3-yl]-carbamic acid tert-butyl ester

The title compound is prepared in an analogous manner as described for example 4a) starting from (3-tert-butyl-benzyl)-[(3R*,4S*,5S*)-5-(3-fluoro-4-nitro-benzyl)-4-hydroxy-1,1-dioxo-hexahydro-1lambda*6*-thiopyran-3-yl]-carbamic acid tert-butyl ester (example 2f)): TLC (hexane-EtOAc 2:1) Rf=0.42; HPLC Rt_{A}=2.57 min; ESIMS [M+H+NH₃]⁺=622.

### b) [(3R,4S,5S)-5-(4-Amino-3-propoxy-benzyl)-4-hydroxy-1,1-dioxo-hexahydro-1lambda*6*-thiopyran-3-yl]-(3-tert-butyl-benzyl)-carbamic acid tert-butyl ester

The title compound is prepared in analogous manner as described for example 2g) starting from (3-tert-butyl-benzyl)-[(3R*,4S*,5S*)-4-hydroxy-5-(4-nitro-3-propoxy-benzyl)-1,1-dioxo-hexahydro-1lambda*6*-thiopyran-3-yl]-carbamic acid tert-butyl ester to yield the title compound as a redish oil. The racemate is separated by preparative HPLC on Chiralpak OD-H with CO₂, MeOH 20% to yield the (3R,4S,5S)-enantiomer (peak 1) with >99% ee as a yellow oil and the (3S,4R,5R)-enantiomer (peak 2) with >98% ee as a redish foam: TLC (hexane-EtOAc 2:1) Rf=0.15; HPLC Rt_{A}=2.05 min; [M+H+NH₃]⁺=597.

### c) (3S,4S,5R)-3-(4-Amino-3-propoxy-benzyl)-5-(3-tert-butyl-benzylamino)-1,1-dioxo-hexahydro-1lambda*6*-thiopyran-4-ol hydrochloride

The title compound is prepared in analogous manner as described for example 2i) starting from [(3R,4S,5S)-5-(4-amino-3-propoxy-benzyl )-4-hydroxy-1,1-dioxo-hexahydro-1lambda*6*-thiopyran-3-yl]-(3-tert-butyl-benzyl)-carbamic acid tert-butyl ester: TLC (CH₂Cl₂-MeOH 19:1) Rf=0.42; HPLC Rt_{A}=1.49 min; ESIMS [M+H]⁺=475; ¹H-NMR (600 MHz, DMSO-d₆): δ 9.95 (s 1H), 9.05 (s, 1H), 7.64 (s, 1H), 7.44 (dt, 1H), 7.37 (m, 2H), 7.31 (d, 1H), 7.02 (s, 1H), 6.83 (d, 1H), 4.25 (m, 2H), 4.05 (m, 2H), 3.85 (dt, 1H), 3.65 (m, 2H), 3.2 (m, 3H), 2.71 (dt, 1H), 2.44 (dd, 1H), 2.10 (m, 1H), 1.76 (m, 2H), 1.29 (s, 9H), 1.01 (s, 3H).

### Example 6

### (3S*,4S*,5R*)-3-(4-Amino-3-chloro-5-propoxy-benzyl)-5-(3-tert-butyl-benzyl-amino)-1,1-dioxo-hexahydro-1lambda*6*-thiopyran-4-ol hydrochloride

### a) [(3R,4S,5S)-5-(4-Amino-3-chloro-5-propoxy-benzyl)-4-hydroxy-1,1-dioxo-hexahydro-1lambda*6*-thiopyran-3-yl]-(3-tert-butyl-benzyl)-carbamic acid tert-butyl ester

A solution of [(3R,4S,5S)-5-(4-amino-3-propoxy-benzyl)-4-hydroxy-1,1-dioxo-hexahydro-1lambda*6*-thiopyran-3-yl]-(3-tert-butyl-benzyl)-carbamic acid tert-butyl ester (0.23 g, 0.39 mmol) and N-chlorosuccinimide (0.52 g, 0.41 mmol) in ACN (5 mL) is heated at 40° C. for 16 h. The reaction mixture is poured onto cold NaHCO₃ solution and the product is extracted with EtOAc. The combined organic layers are washed with brine, dried over MgSO₄ and evaporated. The title compound is obtained by preparative HPLC (Nucleodur C₁₈, 250×19 mm, 30-100% ACN in water+0.1% TFA gradient, 30 min): TLC (toluene-EtOAc 1:1) Rf=0.69; HPLC Rt_{A}=2.51 min; [M+H-Boc]⁺=509, 511.

### b) (3S*,4S*,5R*)-3-(4-Amino-3-chloro-5-propoxy-benzyl)-5-(3-tert-butyl-benzylamino)-1,1-dioxo-hexahydro-1lambda*6*-thiopyran-4-ol hydrochloride

The title compound is prepared in analogous manner as described for example 2i) starting from [(3R,4S,5S)-5-(4-amino-3-chloro-5-propoxy-benzyl)-4-hydroxy-1,1-dioxo-hexahydro-1lambda*6*-thiopyran-3-yl]-(3-tert-butyl-benzyl)-carbamic acid tert-butyl ester: HPLC Rt_{A}=1.91 min; ESIMS [M+H]⁺=509, 511.

### Example 7

### (3S*,4S*,5R*)-3-(4-Amino-3-fluoro-5-propyl-benzyl)-5-(3-tert-butyl-benzyl-amino)-1,1-dioxo-hexahydro-1lambda*6*-thiopyran-4-ol hydrochloride

### a) [(3R,4S,5S)-5-(3-Allyl-4-amino-5-fluoro-benzyl)-4-hydroxy-1,1-dioxo-hexahydro-1lambda*6*-thiopyran-3-yl]-(3-tert-butyl-benzyl)-carbamic acid tert-butyl ester

To a degassed solution of [(3R*,4S*,5S*)-5-(4-amino-3-bromo-5-fluoro-benzyl)-4-hydroxy-1,1-dioxo-hexahydro-1lambda*6*-thiopyran-3-yl]-(3-tert-butyl-benzyl)-carbamic acid tert-butyl ester (example 2h)) (0.1 g, 0.16 mmol) and 2-allyl-4,4,5,5-tetramethyl-[1,3,2]dioxaborolane (0.056 g, 0.32 mmol) in DME-H₂O 10:1 (6 mL) is added under argon K₃PO₄ (0.07 g, 0.32 mmol), Ph₅FcP(tBu)₂ (0.007 g, 0.01 mmol) and Pd₂(dba)₃ (0.005 g, 0.005 mmol) and the mixture is heated for 3 h at 80° C. The mixture is diluted with EtOAc, washed with Na₂CO₃ solution and brine, dried over MgSO₄ and evaporated. The title compound is obtained after purification by flash-chromatography on silica gel (hexane-EtOAc 3:1 to 1:2) as a colorless oil: TLC (hexane-EtOAc 1:1) Rf=0.51; HPLC Rt_{A}=2.36 min; ESIMS [M+H-100]⁺=475.

### b) [(3R*,4S*,5S*)-5-(4-Amino-3-fluoro-5-propyl-benzyl)-4-hydroxy-1,1-dioxo-hexahydro-1lambda*6*-thiopyran-3-yl]-(3-tert-butyl-benzyl)-carbamic acid tert-butyl ester

A solution of [(3R,4S,5S)-5-(3-Allyl-4-amino-5-fluoro-benzyl)-4-hydroxy-1,1-dioxo-hexahydro-1lambda*6*-thiopyran-3-yl]-(3-tert-butyl-benzyl)-carbamic acid tert-butyl ester (0.039 g, 0.064 mmol) in MeOH (6 mL) is hydrogenated over 5% Pd/C (10 mg) at room temperature and 1 mbar. After 2 h the catalyst is filtered off over Celite and the filtrate is evaporated to yield the title compound as a colorless oil: TLC (hexane-EtOAc 1:1) Rf=0.52; HPLC Rt_{A}=2.35 min; ESIMS [M+H-Boc]⁺=477.

### c) (3S*,4S*,5R*)-3-(4-Amino-3-fluoro-5-propyl-benzyl)-5-(3-tert-butyl-benzylamino)-1,1-dioxo-hexahydro-1lambda*6*-thiopyran-4-ol hydrochloride

The title compound is prepared in analogous manner as described for example 2i) starting from [(3R*,4S*,5S*)-5-(4-amino-3-fluoro-5-propyl-benzyl)-4-hydroxy-1,1-dioxo-hexahydro-1lambda*6*-thiopyran-3-yl]-(3-tert-butyl-benzyl)-carbamic acid tert-butyl ester: TLC (CH₂Cl₂-MeOH—AcOH—H₂O 180:20:2:1) Rf=0.74; HPLC Rt_{A}=1.74 min; ESIMS [M+H]⁺=477; ¹H-NMR (400 MHz, CD₃OD): δ 7.62 (s, 1H), 7.54 (dt, 1H), 7.42 (t, 1H), 7.35 (d, 1H), 7.07 (d, 1H), 7.05 (s, 1H), 4.33 (m, 2H), 3.45-3.8 (m, 4H), 3.35 (dd, 1H), 3.20 (t,1H), 2.73 (dt, 1H), 2.70 (t, 2H), 2.51 (dd, 1H), 2.26 (m, 1H), 1.66 (m, 2H), 1.36 (s, 9H), 1.02 (s, 3H).

### Example 8

### (3S,4S,5R)-3-(4-Amino-3-fluoro-5-vinyl-benzyl)-5-(3-tert-butyl-benzylamino)-1,1-dioxo-hexahydro-1lambda*6*-thiopyran-4-ol

### a) [(3R,4S,5S)-5-(4-Amino-3-fluoro-5-vinyl-benzyl)-4-hydroxy-1,1-dioxo-hexahydro-1lambda*6*-thiopyran-3-yl]-(3-tert-butyl-benzyl)-carbamic acid tert-butyl ester

To a degassed solution of [(3R,4S,5S)-5-(4-amino-3-bromo-5-fluoro-benzyl)-4-hydroxy-1,1-dioxo-hexahydro-1lambda*6*-thiopyran-3-yl]-(3-tert-butyl-benzyl)-carbamic acid tert-butyl ester (example 2h)) (0.26 g, 0.42 mmol), Cs₂CO₃ (0.27 g, 0.85 mmol), 2,4,6-trivinyl-cyclotriboroxane pyridine complex (0.15 g, 0.64 mmol) and P(tBu)₃ (0.02 g, 0.06 mmol) in dioxane is added under argon the Pd₂(dba)₃ (0.012 g, 0.013 mmol) and the mixture is heated at reflux for 1 h. The title compound is obtained after flash-chromatography on silica gel (cyclohexane-EtOAc 1:1) as a white foam: ESIMS [M-Boc]⁺=461; HPLC Rt_{D}=6.83 min.

### b) (3S,4S,5R)-3-(4-Amino-3-fluoro-5-vinyl-benzyl)-5-(3-tert-butyl-benzylamino)-1,1-di-oxo-hexahydro-1lambda*6*-thiopyran-4-ol

A solution of [(3R,4S,5S)-5-(4-amino-3-fluoro-5-vinyl-benzyl )-4-hydroxy-1,1-dioxo-hexahydro-1lambda*6*-thiopyran-3-yl]-(3-tert-butyl-benzyl)-carbamic acid tert-butyl ester (0.19 g, 0.34 mmol) is treated with 1M HCl in Et₂O (3.39 mL, 3.39 mmol) at 0° C. The mixture is stirred overnight. After concentration, the residue is diluted with EtOAc and quenched with saturated NaHCO₃, dried over Na₂SO₄, filtered and concentrated. The title compound is obtained after flash-chromatography on silica gel (cyclohexane-EtOAc 1:1) as a yellow foam: ESIMS [M+H]⁺=461; ¹⁹F-NMR (376 MHz, CDCl₃): δ−144 (s, 1F).

### Example 9

### (3S,4S,5R)-3-(4-Amino-3-ethyl-5-fluoro-benzyl)-5-(3-tert-butyl-benzylamino)-1,1-dioxo-hexahydro-1lambda*6*-thiopyran-4-ol

A solution of (3S,4S,5R)-3-(4-amino-3-fluoro-5-vinyl-benzyl )-5-(3-tert-butyl-benzylamino)-1,1-dioxo-hexahydro-1lambda*6*-thiopyran-4-ol (0.14 g, 0.3 mmol) is hydrogenated (1 atm H₂) in EtOH (10 mL) at 25° C. over 10% Pd—C (10 mg) for 15 h. The catalyst is filtered off over Celite and after evaporation of the solvent the title compound is obtained as a white powder: HPLC Rt_{D}=5.82 min; ESIMS [M+H]⁺=463; ¹⁹F-NMR (376 MHz, CD₃OD): δ−144 (s, 1F).

### Example 10

### (3S*,4S*,5R*)-3-(4-Amino-3-fluoro-5(Z)-propenyl-benzyl)-5-(3-tert-butyl-benzylamino)-1,1-dioxo-hexahydro-1lambda*6*-thiopyran-4-ol hydrochloride and (3S*,4S*,5R*)-3-(4-Amino-3-fluoro-5(E)-propenyl-benzyl)-5-(3-tert-butyl-benzylamino)-1,1-dioxo-hexahydro-1lambda*6*-thiopyran-4-ol hydrochloride

### a) {(3R*,4S*,5S*)-5-[4-Amino-3-fluoro-5-(Z)-propenyl-benzyl]-4-hydroxy-1,1-dioxo-hexahydro-1lambda*6*-thiopyran-3-yl}-(3-tert-butyl-benzyl)-carbamic acid tert-butyl ester and {(3R*,4S*,5S*)-5-[4-Amino-3-fluoro-5-(E)-propenyl-benzyl]-4-hydroxy-1,1-dioxo-hexahydro-1lambda*6*-thiopyran-3-yl}-(3-tert-butyl-benzyl)-carbamic acid tert-butyl ester

To a solution of [(3R*,4S*,5S*)-5-(4-amino-3-bromo-5-fluoro-benzyl)-4-hydroxy-1,1-dioxo-hexahydro-1lambda*6*-thiopyran-3-yl]-(3-tert-butyl-benzyl)-carbamic acid tert-butyl ester (example 2h)) (0.1 g, 0.163 mmol) in DME, CsF (0.149 g, 0.978 mmol), trans-propenyl boronic acid (56 mg, 0.652 mmol) and Pd(PPh₃)₄ (11 mg, 0.010 mmol) are added under argon. The mixture is heated in a microwave oven at 140° C. for 30 min. The title compound is obtained after purification by preparative HPLC (Sun Five C₁₈ OBD 5 μm, 100×30, 5-100% ACN in water+0.1% TFA gradient, 25 min) as a mixture of isomers: TLC (cyclohexane-EtOAc 4:1) Rf=0.24; HPLC Rt_{D}=6.06 min; ESIMS [M+H-Boc]⁺=475.

### b) (3S*,4S*,5R*)-3-(4-Amino-3-fluoro-5(Z)-propenyl-benzyl)-5-(3-tert-butyl-benzylamino)-1,1-dioxo-hexahydro-1lambda*6*-thiopyran-4-ol hydrochloride and (3S*,4S*,5R*)-3-(4-Amino-3-fluoro-5(E)-propenyl-benzyl)-5-(3-tert-butyl-benzylamino)-1,1-dioxo-hexahydro-1lambda*6*-thiopyran-4-ol hydrochloride

The title compound is prepared in analogous manner as described for example 2i) starting from {(3R,4S,5S)-5-[4-amino-3-fluoro-5-(Z)-propenyl-benzyl]-4-hydroxy-1,1-dioxo-hexahydro-1lambda*6*-thiopyran-3-yl}-(3-tert-butyl-benzyl)-carbamic acid tert-butyl ester and {(3R,4S,5S)-5-[4-amino-3-fluoro-5-(E)-propenyl-benzyl]-4-hydroxy-1,1-dioxo-hexahydro-1lambda*6*-thiopyran-3-yl}-(3-tert-butyl-benzyl)-carbamic acid tert-butyl ester. TLC (CH₂Cl₂—MeOH 95-5) Rf=0.1; HPLC Rt_{D}=4.75 min; ESIMS [M+H]⁺=475.

### Examples 11a -11e

The compounds listed in Table 2 can be prepared by a procedure analogous to that used in example 8 or example 9.

TABLE 2

| | | HPLC | |
|---|---|---|---|
| | | Rt [min] | |
| Example | Compound | Method | ESIMS |
| 11a | (3S*,4S*,5R*)-3-[4-Amino-3-fluoro-5-(2-methyl- | 2.35 C | [M − H]⁺ = 489 |
| | propenyl)-benzyl]-5-(3-tert-butyl-benzylamino)-1,1- | | |
| | dioxo-hexahydro-1lambda*6*-thiopyran-4-ol | | |
| 11b | (3S*,4S*,5R*)-3-(4-Amino-3-fluoro-5-isobutyl-benzyl)- | 4.47 D | [M − H]⁺ = 475 |
| | 5-(3-tert-butyl-benzylamino)-1,1-dioxo-hexa- | | |
| | hydro-1lambda*6*-thiopyran-4-ol hydrochloride | | |
| 11c | (3S*,4S*,5R*)-3-[4-Amino-3-(1,2-dimethyl-propenyl)- | 2.46 C | [M − H]⁺ = 503 |
| | 5-fluoro-benzyl]-5-(3-tert-butyl-benzylamino)- | | |
| | 1,1-dioxo-hexahydro-1lambda*6*-thiopyran-4-ol | | |
| | hydrochloride | | |
| 11d | (3S*,4S*,5R*)-3-(4-Amino-3-fluoro-5-furan-2-yl- | 2.27 C | [M − H]⁺ = 501 |
| | benzyl)-5-(3-tert-butyl-benzylamino)-1,1-dioxo-hexahydro- | | |
| | 1lambda*6*-thiopyran-4-ol hydrochloride | | |
| 11e | (3S*,4S*,5R*)-3-(4-Amino-3-fluoro-5-furan-3-yl- | 2.22 C | [M − H]⁺ = 501 |
| | benzyl)-5-(3-tert-butyl-benzylamino)-1,1-dioxo-hexahydro- | | |
| | 1lambda*6*-thiopyran-4-ol hydrochloride | | |

### Example 12

### (3S*,4S*,5R*)-3-(4-Amino-3-bromo-benzyl)-5-(3-tert-butylbenzylamino)-tetrahydro-thiopyran-4-ol fumarate

### a) 3-(3-Bromo-4-nitro-benzyl)-5-tert-butoxycarbonylamino-4-oxo-tetrahydro-thiopyran-3-carboxylic acid allyl ester

The title compound is prepared in analogous manner as described for example 1 c) starting from 5-tert-butoxycarbonylamino-4-oxo-tetrahydro-thiopyran-3-carboxylic acid allyl ester and 4-nitro-3-bromobenzylbromide. The product mixture obtained as a light yellow resin is suitable for use in the next step: TLC (hexane-EtOAc 3:1) Rf=0.38 and 46; ESIMS [M+H+NH₃]⁺=548, 546.

### b) [(3R*,5S*)-5-(3-Bromo-4-nitro-benzyl)-4-oxo-tetrahydro-thiopyran-3-yl]-carbamic acid tert-butyl ester

The title compound is prepared in analogous manner by decarboxylation and equilibration as described for example 1d): TLC (hexane-EtOAc 3:1) Rf=0.40; ESIMS [M+H+NH₃]⁺=462, 464.

### c) [(3R*,4S*,5S*)-5-(3-Bromo-4-nitro-benzyl)-4-hydroxy-tetrahydro-thiopyran-3-yl]-carbamic acid tert-butyl ester

The title compound is prepared in analogous manner as described for example 1e) using LiAlH₄ in THF at −70° C. The mixture of diastereoisomers is separated by flash-chromatograpy on silica gel (hexane-EtOAc 7:3) to yield the undesired [(3R*,4R*,5S*)-diastereoisomer and the desired [(3R*,4S*,5S*)-diastereoisomer as yellow brownish crystals: TLC (hexane-EtOAc 7:3) Rf=0.17; HPLC Rt_{B}=13.51 min (without TFA); ESIMS [M+H-56 (isobutylene)]⁺=393, 391.

### d) (3R*,4S*,5S*)-3-Amino-5-(3-bromo-4-nitro-benzyl)-tetrahydro-thiopyran-4-ol

[(3R*,4S*,5S*)-5-(3-Bromo-4-nitro-benzyl)-4-hydroxy-tetrahydro-thiopyran-3-yl]-carbamic acid tert-butyl ester (0.201 g, 0.45 mmol) in a mixture of CH₂Cl₂ (4 ml) and TFA (0.4 mL) is stirred for 1.5 h at 25° C. The mixture is evaporated and dried to yield the title compound as light yellow brownish foam: ESIMS [M+H-Boc]⁺=349, 347.

### e) (3S*,4S*,5R*)-3-(3-Bromo-4-nitro-benzyl)-5-(3-tert-butyl-benzylamino)-tetrahydro-thiopyran-4-ol

The title compound is prepared in analogous manner as described for example 1h): TLC (toluene-ETA 95:5) Rf=0.30; ESIMS [M+H]⁺=495 and 493.

### f) (3S*,4S*,5R*)-3-(4-Amino-3-bromo-benzyl)-5-(3-tert-butylbenzylamino)-tetrahydro-thiopyran-4-ol fumarate

(3S*,4S*,5R*)-3-(3-Bromo-4-nitro-benzyl)-5-(3-tert-butyl-benzylamino)-tetrahydro-thiopyran-4-ol (0.133 g, 0.27 mmol) is dissolved in MeOH (1.3 mL), THF (1.3 mL) and water (1.3 mL). Sodium dithionite (0.115 g, 0.54 mmol) is added in portions at 60° C. over 1 h. After stirring at 60° C. for 1 h the mixture is quenched with ice-water and aqueous NaOH and diluted with EtOAc. The solution is washed with water, saturated NaHCO₃ solution and brine, dried over Na₂SO₄ and evaporated. The title compound is obtained after preparative TLC-chromatography (PSC 20×20 cm, 1 mm; silica 60 Merck) on silica gel (toluene-EtOAc 95:5) as a white foam after transformation into the fumarate salt with fumaric acid in tBuOMe-MeOH: TLC (toluene-ETA 95:5) Rf=0.25; HPLC Rt_{B}=12.62 min; ESIMS [M+H]⁺=465, 463.

### Example 13

### (3S*,4S*,5R*)-3-(4-Amino-3-bromo-benzyl)-5-(3-tert-butyl-benzylamino)-1-oxo-tetrahydro-thiopyran-4-ol

### a) (3R*,4S*,5S*)-5-(4-Amino-3-bromo-benzyl)-4-hydroxy-tetrahydro-thiopyran-3-yl]-carbamic acid tert-butyl ester

The title compound is prepared from [(3R*,4S*,5S*)-5-(3-bromo-4-nitro-benzyl)-4-hydroxy-tetrahydro-thiopyran-3-yl]-carbamic acid tert-butyl ester (example 12c)) and sodium-dithionite (4 equivalents) at 40° C.: TLC (toluene-ETA 95:5) Rf=0.20; ESIMS [M+H]⁺=363, 361 (−56, isobutylene).

### b) [(3R*,4S*,5S*)-5-(4-Amino-3-bromo-benzyl)-4-hydroxy-1-oxo-tetrahydro-thiopyran-3-yl]-carbamic acid tert-butyl ester

(3R*,4S*,5S*)-5-(4-Amino-3-bromo-benzyl )-4-hydroxy-tetrahydro-thiopyran-3-yl]-carbamic acid tert-butyl ester (0.192 g, 0.46 mmol) is dissolved in MeOH (14 mL) and water (1.9 mL). Sodium metaperiodate (0.098 g, 0.46 mmol) is added in small portions over 1 h at room temperature. The mixture is stirred at room temperature for 1 day and evaporated. The residue is diluted with EtOAc and Na₂CO₃ solution, washed with water and brine, dried over Na₂SO₄ and evaporated to yield a mixture of isomeric sulfoxides as a pink foam: TLC (EtOAc-MeOH 95:5) Rf=0.24 and 0.29; ESIMS [M+H]⁺=435, 433.

### c) (3R*,4S*,5S*)-3-Amino-5-(4-amino-3-bromo-benzyl)-1-oxo-tetrahydro-thiopyran-4-ol

The title compound is prepared in analogous manner as described for example 12d) from [(3R*,4S*,5S*)-5-(4-amino-3-bromo-benzyl)-4-hydroxy-1-oxo-tetrahydro-thiopyran-3-yl]-carbamic acid tert-butyl ester and TFA to yield a mixture of isomeric sulfoxides: ESIMS [M+H]⁺=335, 333.

### d) (3S*,4S*,5R*)-3-(4-Amino-3-bromo-benzyl)-5-(3-tert-butyl-benzylamino)-1-oxo-tetra-hydro-thiopyran-4-ol

The title compound is prepared in analogous manner as described for example 1h) from (3R*,4S*,5S*)-3-amino-5-(4-amino-3-bromo-benzyl)-1-oxo-tetrahydro-thiopyran-4-ol (TFA salt). The mixture of diastereoisomeric sulfoxides is separated by flash-chromatograpy on silica gel (EtOAc-ETA 95:5) to yield the syn and anti diastereoisomers: Syn-diastereoisomer: TLC (toluene-ETA 9:1) Rf=0.25; HPLC Rt_{B}=9.10 min; ESIMS [M+H]⁺=481, 479; Anti-diastereoismer: TLC (toluene-ETA 9:1) Rf=0.20; HPLC Rt_{B}=9.78 min; ESIMS [M+H]⁺=481, 479.

### Example 14

### (3S*,4S*,5R*)-3-(4-Amino-3-trifluoromethoxy-benzyl)-5-(3-tert-butyl-benzylamino)-1,1-dioxo-hexahydro-1lambda*6*-thiopyran-4-ol hydrochloride

### a) {4-[(3S,4S,5R)-5-(3-tert-Butyl-benzylamino)-4-hydroxy-1,1-dioxo-hexahydro-1lambda*6*-thiopyran-3-yl methyl]-2-trifluoromethoxy-phenyl}-carbamic acid benzyl ester

The title compound is prepared in analogous manner as described for examples 1c) to 1h), starting from 5-tert-butoxycarbonylamino-4-oxo-tetrahydro-thiopyran-3-carboxylic acid allyl ester and (4-bromomethyl-2-trifluoromethoxy-phenyl)-carbamic acid benzyl ester: TLC (toluene-EtOAc 1:1) Rf=0.28; ESIMS [M+H]⁺=635.

### b) (3S*,4S*,5R*)-3-(4-Amino-3-trifluoromethoxy-benzyl)-5-(3-tert-butyl-benzylamino)-1,1-dioxo-hexahydro-1lambda*6*-thiopyran-4-ol hydrochloride

A solution of {4-[(3S,4S,5R)-5-(3-tert-butyl-benzylamino)-4-hydroxy-1,1-dioxo-hexahydro-1lambda*6*-thiopyran-3-ylmethyl]-2-trifluoromethoxy-phenyl}-carbamic acid benzyl ester (0.45 g, 0.70 mmol) in MeOH (10 mL) is hydrogenated over 10% Pd/C (25 mg) at room temperature and 1 mbar. After 1.5 h the catalyst is filtered off over Celite and the filtrate is evaporated. The free base is converted into the hydrochloride salt with 1N HCl in Et₂O to yield the title compound after crystallization from diisopropylether as a colorless solid: HPLC Rt_{A}=1.75 min; ESIMS [M+H-Boc]⁺=501; 1H-NMR (600 MHz, DMSO-d₆+TFA): δ 9.05 (s, 1H), 7.64 (s, 1H), 7.44 (m, 1H), 7.37 (m, 2H), 7.10 (s, 1H), 7.04 (m, 2H), 4.23 (m, 2H), 3.84 (dt, 1H), 3.62 (m, 2H), 3.2 (m, 2H), 3.11 (d, 1H), 2.74 (dt, 1H), 2.45 (dd, 1H), 2.04 (m, 1H), 1.76 (m, 2H), 1.28 (s, 9H).

### c) 4-Benzyloxycarbonylamino-3-trifluoromethoxy-benzoic acid methyl ester

To a solution of 4-amino-3-trifluoromethoxy-benzoic acid methyl ester (4.9 g, 20.8 mmol) and NaHCO₃ (5.24 g, 62.4 mmol) in dioxane-water 5:1 (90 mL) is added at 0-5° C. benzyl chloroformate (4.4 mL, 31.3 mmol). After addition the reaction mixture is stirred at 25° C. for 16 h. The reaction mixture is diluted with CH₂Cl₂ and washed with water, dried over MgSO₄ and evaporated. The title compound is obtained after purification by flash-chromatography on silica gel (hexane-EtOAc 30:1 to 10:1) as a colorless oil: TLC (hexane-EtOAc 4:1) Rf=0.52; ESIMS [M−H]⁻=368.

### d) (4-Hydroxymethyl-2-trifluoromethoxy-phenyl)-carbamic acid benzyl ester

To a solution of LiAlH₄ (1.29 g, 34.1 mmol) in THF (100 mL) is added at 0-5° C. a solution of 4-benzyloxycarbonylamino-3-trifluoromethoxy-benzoic acid methyl ester (4.2 g, 11.37 mmol) dissolved in THF (30 mL). After stirring for 2 h at 0-5° C. a 10% aqueous solution of Na₂SO₄ is slowly added under cooling and after stirring for 1 h the mixture is filtered through Celite. The solvents are evaporated and the residual oil is taken up in EtOAc, washed with brine, dried over MgSO₄ and evaporated. The title compound is obtained as a yellow oil suitable for use in the next step: TLC (EtOAc) Rf=0.36; ESIMS [M−H]⁻=340.

### e) (4-Bromomethyl-2-trifluoromethoxy-phenyl)-carbamic acid benzyl ester

To a solution of (4-hydroxymethyl-2-trifluoromethoxy-phenyl)-carbamic acid benzyl ester (3.7 g, 10.8 mmol) in dioxane (90 mL) is added N-bromosuccinimide (2.89 g, 16.3 mmol) and PPh₃ (4.25 g, 16.3 mmol) and the reaction mixture is heated at 55° C. for 10 min. The solvent is evaporated and the title compound is obtained after purification by flash-chromatography on silica gel (hexane-EtOAc 50:1) as a light yellow solid: TLC (hexane-EtOAc 9:1) Rf=0.73; ¹H-NMR (400 MHz, CDCl₃): δ 8.22 (d, 1H), 7.2-7.4 (m, 7H), 7.02 (s, 1H), 5.19 (s, 2H), 4.44 (s, 2H).

### Example 15

### (3S*,4S*,5R*)-3-[4-Amino-3-(,1-difluoro-ethyl)-5-fluoro-benzyl]-5-(3-tert-butyl-benzylamino)-1,1-dioxo-hexahydro-1lambda*6*-thiopyran-4-ol

The title compound is prepared in analogous manner as described for examples 1c) to 1i), starting from 5-tert-butoxycarbonylamino-4-oxo-tetrahydro-thiopyran-3-carboxylic acid allyl ester and 5-bromomethyl-1-(1,1-difluoro-ethyl)-3-fluoro-2-nitro-benzene: HPLC Rt_{D}=4.88 min; ESIMS [M+H]⁺=499; ¹⁹F-NMR (376 MHz, CDCl₃): δ−88 (s, 2F), −134 (s, 1F).

### a) 4-Amino-3-bromo-5-fluoro-benzoic acid methyl ester

To a solution of 4-amino-3-fluoro-benzoic acid methyl ester (10.0 g, 57.9 mmol) in ACN (280 mL) is added water (60 mL) and NaBr (6.02 g, 57.9 mmol). To this solution is slowly added a solution of oxone (35.9 g, 57.9 mmol) in water (80 mL). After stirring for 2 h at 25° C., the mixture is extracted with EtOAc. Combined extracts are washed with NaS₂O₃ solution and brine, dried over MgSO₄ and evaporated. The title compound is obtained after crystallization from diisopropylether as a beige solid: TLC (toluene-EtOAc 1:1) Rf=0.67; HPLC Rt_{A}=1.66 min; ESIMS [M−H]⁻=246 and 248; ¹H-NMR (400 MHz, CDCl₃): δ 7.96 (d, 1H), 7.63 (dd, 1H), 3.84 (s, 3H).

### b) 3-Bromo-5-fluoro-4-nitro-benzoic acid methyl ester

To a solution of 50% H₂O₂ (15.5 mL, 268 mmol) in CH₂Cl₂ (150 mL) at 0° C. is added drop-wise trifluoroacetic anhydride (43.6 mL, 313 mmol). The solution is warmed to 25° C. and 4-amino-3-bromo-5-fluoro-benzoic acid methyl ester (10 g, 40.3 mmol) dissolved in CH₂Cl₂ (50 mL) is added dropwise. After stirring for 1 h at 45° C., the mixture is cooled to 0° C. and Na₂SO₃ is added slowly. The organic layer is extracted with EtOAc (3×50 mL). Combined extracts are washed with brine, dried over MgSO₄ and evaporated. The title compound is obtained after purification by flash-chromatography on silica gel (cyclohexane-EtOAc 9:1) as a light yellow solid: HPLC Rt_{D}=6.76 min; ¹H-NMR (400 MHz, CDCl₃): δ 8.15 (d, 1H), 7.85 (dd, 1H), 3.96 (s, 3H); ¹⁹F-NMR (376 MHz, CDCl₃): δ−115 (s, 1F).

### c) 3-Fluoro-4-nitro-5-trimethylsilanylethynyl-benzoic acid methyl ester

To a solution of 3-bromo-5-fluoro-4-nitro-benzoic acid methyl ester (10 g, 36 mmol) in NEt₃ (60 mL) is added Pd(PPh₃)₂Cl₂ (1.01 g, 1.44 mmol) and the resulting suspension is stirred for 10 min. Ethynyltrimethylsilane (4.47 mL, 53.9 mmol) and CuI (1.37 g, 7.19 mmol) are added. The mixture is stirred for 6 h at 25° C. The title compound is obtained after purification by flash-chromatography on silica gel (cyclohexane-EtOAc 9:1): HPLC Rt_{C}=5.36 min; ESIMS [M+H₂O]⁺=313; ¹H-NMR (400 MHz, CDCl₃): δ 8.06 (d, 1H), 7.85 (dd, 1H), 3.95 (s, 3H); ¹⁹F-NMR (376 MHz, CDCl₃): δ−121 (s, 1F).

### d) 3-Acetyl-5-fluoro-4-nitro-benzoic acid methyl ester

To a solution of 3-fluoro-4-nitro-5-trimethylsilanylethynyl-benzoic acid methyl ester in 80% aqueous acetone (233 mL, 0.05M) are added HgSO₄ (34.6 g, 116 mmol) and sulfuric acid (1.24 mL, 23.3 mmol). The reaction mixture is stirred at 60° C. overnight. The title compound is obtained after purification by flash-chromatography on silica gel (cyclohexane-EtOAc 1:1): HPLC Rt_{C}=1.33 min; ESIMS [M+H₂O]⁺=259; ¹H-NMR (400 MHz, CDCl₃): δ 8.25 (d, 1H), 8.07 (dd, 1H), 4.02 (s, 3H), 2.65 (s, 3H); ¹⁹F-NMR (376 MHz, CDCl₃): δ−122 (s, 1F)

### e) 3-(1,1-Difluoro-ethyl)-5-fluoro-4-nitro-benzoic acid methyl ester

3-Acetyl-5-fluoro-4-nitro-benzoic acid methyl ester (2.11 g, 7.96 mmol) is mixed with 50% deoxo-Fluor in THF (13.7 mL, 31.8 mmol). The solution is stirred for 1 day at 80° C. The title compound is obtained after purification by flash-chromatography on silica gel (cyclohexane-EtOAc 4:1): HPLC Rt_{C}=4.68 min; ¹H-NMR (400 MHz, CDCl₃): δ 8.08 (d, 1H), 7.97 (dd, 1H), 4.00 (s, 3H), 2.04 (t, 3H); ¹⁹F-NMR (377 MHz, CDCl₃): δ−87 (s, 2F), −122 (s, 1F).

### f) [3-(1,1-Difluoro-ethyl)-5-fluoro-4-nitro-phenyl]-methanol

To a solution of 3-(1,1-difluoro-ethyl)-5-fluoro-4-nitro-benzoic acid methyl ester (0.98 g, 3.72 mmol) in THF (40 mL) at 0° C. under argon is added a solution of 1 M DIBAL in hexane (12.4 mL, 12.4 mmol) and is stirred for 1 h at 0° C. The mixture is poured into a solution of 1M potassium sodium tartrate (40 mL, 40 mmol) and the solution is stirred for 30 min. The organic layer is extracted with EtOAc (30×30 mL). The combined extracts are washed with brine, dried over MgSO₄ and evaporated. The title compound is used as it in the next reaction: HPLC Rt_{C}4.13 min; ESIMS [M+H₂O]⁺=253; ESIMS [M−H]⁻=234; ¹H-NMR (400 MHz, CDCl₃): δ 7.38 (dd, 1H), 7.35 (d, 1H), 4.80 (s, 2H), 2.02 (t, 3H); ¹⁹F-NMR (376 MHz, CDCl₃): δ−87 (s, 2F), −123 (s, 1F).

### g) 5-Bromomethyl-1-(1,1-difluoro-ethyl)-3-fluoro-2-nitro-benzene

To a solution of N-bromosaccharin (1.74 g, 6.38 mmol) and PPh₃ (1.67 g, 6.38 mmol) in CH₂Cl₂ (50 mL) is added [3-(1,1-difluoro-ethyl)-5-fluoro-4-nitro-phenyl]-methanol (0.5 g, 2.13 mmol) dissolved in CH₂Cl₂ (5 mL). The solution is stirred for 3 h at 25° C. The title compound is obtained after purification by flash-chromatography on silica gel (cyclohexane-EtOAc 4:1): HPLC Rt_{C}=4.79 min; ESIMS [M+H₂O]⁻=314/316; ¹H-NMR (400 MHz, CDCl₃): δ 7.40 (dd, 1H), 7.38 (d, 1H), 4.44 (s, 2H), 2.02 (t, 3H); ¹⁹F-NMR (376 MHz, CDCl₃): δ−87 (s, 2F), −122 (s, 1F).

### Example 16

### 1-{2-Amino-5-[(3S*,4S*,5R*)-5-(3-tert-butyl-benzylamino)-4-hydroxy-1,1-dioxo-hexahydro-1lambda*6*-thiopyran-3-yl methyl]-3-fluoro-phenyl}-ethanone

The compound of example 15 is treated with TFA (2 mL) for 1 h to give 1-{2-amino-5-[(3S*,4S*, 5R*)-5-(3-tert-butyl-benzylamino)-4-hydroxy-1,1-dioxo-hexahydro-1lambda*6*-thiopyran-3-ylmethyl]-3-fluoro-phenyl}-ethanone. HPLC Rt_{D}=4.68 min; ESIMS [M−H]⁺=477; ESIMS [M−H]⁻=475; ¹⁹F-NMR (376 MHz, CDCl₃): δ−122 (s, 1F).

### Example 17

### (3S,4S,5R)-3-(4-Amino-3-butyl-5-fluoro-benzyl)-5-(3-tert-butyl-benzyl-amino)-1,1-dioxo-hexahydro-1lambda*6*-thiopyran-4-ol

### a) 4-Amino-3-fluoro-5-iodo-benzoic acid methyl ester

To a suspension of 4-amino-3-fluoro-benzoic acid methyl ester (25.0 g, 140 mmol) in EtOH-water 3:2 (200 mL) and CaCO₃ (25.1 g, 246 mmol) is added in portions iodine monochloride (10.8 mL, 210 mmol) at 25° C. The mixture is stirred for 48 h at ambient temperature and then slowly diluted with a large amount of NaHCO₃ solution. The organic solvent is removed under reduced pressure and the aqueous phase is extracted with EtOAc. The combined extracts are washed with water and brine, dried over MgSO₄ and evaporated. The crude product is triturated with diisopropylether, filtered off and dried to afford the title compound as a brownish solid: TLC (toluene) Rf=0.29; HPLC Rt_{A}=1.74 min; ESIMS [M−H]⁻=294; ¹H-NMR (400 MHz, CDCl₃): δ 8.13 (d, 1H), 7.62 (dd, 1H), 3.84 (s, 3H).

### b) 4-Amino-3-but-1-ynyl-5-fluoro-benzoic acid methyl ester

To a degassed solution of 4-amino-3-fluoro-5-iodo-benzoic acid methyl ester (9.3 g, 30.5 mmol) in NEt₃ (150 mL) are added but-1-yne (3.6 g, 51 mmol), CuI (0.18 g, 0.93 mmol) and bis(triphenylphosphine)palladium(II)dichloride (0.664 g, 0.927 mmol) and the reaction mixture is stirred for 2 h at 25° C. The reaction mixture is evaporated and the residue is taken up in EtOAc and washed with NaH₂PO₄ and NaHCO_{3.} solution, dried over MgSO₄ and evaporated. The title compound is obtained after purification by flash-chromatography on silica gel (hexane-EtOAc 3:1) as a yellow crystalline solid: TLC (hexane-EtOAc 3:1) Rf=0.46; HPLC Rt_{A=}2.08 min; ¹H-NMR (400 MHz, CDCl₃): δ 7.78 (d, 1H), 7.58 (dd, 1H), 4.60 (s, 2H), 3.84 (s, 3H), 2.45 (t, 2H), 1.66 (m, 2H), 1.07 (t, 3H).

### c) 4-Amino-3-butyl-5-fluoro-benzoic acid methyl ester

A solution of 4-amino-3-but-1-ynyl-5-fluoro-benzoic acid methyl ester (4.8 g, 20.0 mmol) in MeOH (100 mL) is hydrogenated over 10% Pd/C (0.5 g) at 25° C. and 1 mbar. After 8 h the catalyst is filtered off over Celite and the filtrate is evaporated to yield the title compound as a colorless solid: TLC (hexane-EtOAc 3:1) Rf=0.44; HPLC Rt_{A}=2.14 min; ESIMS [M+H]⁺=240.

### d) (4-Amino-3-butyl-5-fluoro-phenyl)-methanol

To a solution of 4-amino-3-butyl-5-fluoro-benzoic acid methyl ester (6.76 g, 30 mmol) in anhydrous THF (150 mL) is added under argon at 0-5° C. LiAlH₄ (1.61 g, 40 mmol) in portions. The reaction mixture is stirred overnight at 25° C. The reaction mixture is added slowly to cold aqueous 1N HCl and the product is extracted with EtOAc. Combined extracts are washed with NaHCO₃ solution and brine, dried over MgSO₄ and evaporated. The title compound is obtained after purification by flash-chromatography on silica gel (hexane-EtOAc 3:1) as a yellow crystals: TLC (hexane-EtOAc 3:1) Rf=0.20; HPLC Rt_{A}=0.99 min; ESIMS [M+H]⁺=198; ¹H-NMR (400 MHz, CDCl₃): δ 6.84 (m, 2H), 4.52 (d, 2H), 3.64 (s, 2H), 2.45 (t, 2H), 1.60 (m, 2H), 1.42 (m, 2H), 0.97 (t, 3H).

### e) (2-Butyl-6-fluoro-4-hydroxymethyl-phenyl)-carbamic acid benzyl ester

To a solution of (4-amino-3-butyl-5-fluoro-phenyl)-methanol (8.14 g, 40.8 mmol) and K₂CO₃ (17.1 g, 122.6 mmol) in dioxane-water 3:1 (150 mL) is added at 0-5° C. benzyl chloroformate (8.3 mL, 53 mmol). After addition the reaction mixture is stirred at 25° C. for 2 h. The reaction mixture is diluted with 1N NaOH and evaporated. The aqueous phase is extracted with EtOAc and the combined extracts are washed with brine, dried over MgSO₄ and evaporated. The title compound is obtained after purification by flash-chromatography on silica gel (hexane-EtOAc 4:1 to 1:1) as a white solid: TLC (hexane-EtOAc 1:1) Rf=0.47; HPLC Rt_{A}=1.95 min; ESIMS [M+H+NH3]⁺=349.

### f) (4-Bromomethyl-2-butyl-6-fluoro-phenyl)-carbamic acid benzyl ester

To a solution of PBr₃ (13.09 g, 47.4 mmol) in Et₂O (150 mL) is added under argon at 0-5° C. a solution of (2-butyl-6-fluoro-4-hydroxymethyl-phenyl)-carbamic acid benzyl ester (12.2 g, 36 mmol) in Et₂O (100 mL) within 0.5 h. The reaction mixture is stirred for 16 h at 25° C. After addition of MeOH (15 mL) the solvents are evaporated and the residue is taken up in EtOAc and washed with cold water and NaHCO₃ solution, dried over MgSO₄ and evaporated. The title compound is obtained as white crystals suitable for use in the next step: TLC (hexane-EtOAc 3:1) Rf=0.49; HPLC Rt_{A}=2.37 min; ¹H-NMR (400 MHz, CDCl₃): δ 7.36 (m, 5H), 7.04 (m, 2H), 5.19 (s, 2H), 4.40 (s, 2H), 2.58 (t, 2H), 1.54 (m, 2H), 1.33 (m, 2H), 0.91 (t, 3H).

### g) 3-(4-Benzyloxycarbonylamino-3-butyl-5-fluoro-benzyl)-5-tert-butoxycarbonylamino-4-oxo-tetrahydro-thiopyran-3-carboxylic acid allyl ester

To a solution of 5-tert-butoxycarbonylamino-4-oxo-tetrahydro-thiopyran-3-carboxylic acid allyl ester (8.0 g, 24.86 mmol) in acetone (200 mL) is added K₂CO₃ (5.26 g, 37.3 mmol) and (4-bromomethyl-2-butyl-6-fluoro-phenyl)-carbamic acid benzyl ester (10.8 g, 26.1 mmol) and the mixture is stirred at 25° C. for 16 h. After dilution with water the product is extracted with EtOAc. Combined extracts are washed with brine, dried over MgSO₄ and evaporated. The product obtained as a light yellow solid is suitable for use in the next step: TLC (toluene-EtOAc 4:1) Rf=0.60; HPLC Rt_{A}=2.61 min; ESIMS [M+H+NH₃]⁺=646.

### h) [(3R*,5S*)-5-(4-Benzyloxycarbonylamino-3-butyl-5-fluoro-benzyl)-4-oxo-tetrahydro-thiopyran-3-yl]-carbamic acid tert-butyl ester

To a degassed solution of 3-(4-benzyloxycarbonylamino-3-butyl-5-fluoro-benzyl)-5-tert-butoxycarbonylamino-4-oxo-tetrahydro-thiopyran-3-carboxylic acid allyl ester (15.5 g, 23.4 mmol) and morpholine (4.4 mL, 49.2 mmol) in THF (200 mL) is added under argon Pd(PPh₃)₄ (0.285 g, 0.234 mmol) and the mixture is stirred overnight at 25° C. The reaction mixture is evaporated and the residual product is taken up in EtOAc, washed with cold NaH₂PO₄ solution and brine, dried over MgSO₄ and evaporated. The title compound is obtained after equilibration with a small amount of DBU and crystallization from diisopropylether as single diastereoisomer: TLC (toluene-EtOAc 4:1) Rf=0.63; HPLC Rt_{A}=2.54 min; ESIMS [M+H+NH₃]⁺=562; ¹H-NMR (400 MHz, CDCl₃): δ 7.37 (m, 5H), 6.78 (m, 2H), 6.0 (s, 1H), 5.75 (d, 1H), 5.19 (s, 2H), 4.54 (m, 1H), 3.40 (m, 1H), 3.17 (dd, 1H), 3.03 (m, 1H), 2.82 (ddd, 1H), 2.4-2.7 (m, 5H), 1.44 (m, 2H), 1.41 (s, 9H), 1.32 (m, 2H), 0.87 (t, 3H).

### i) [(3R*,4S*,5S*)-5-(4-Benzyloxycarbonylamino-3-butyl-5-fluoro-benzyl)-4-hydroxy-tetra-hydro-thiopyran-3-yl]-carbamic acid tert-butyl ester

To a solution of [(3R*,5S*)-5-(4-Benzyloxycarbonylamino-3-butyl-5-fluoro-benzyl)-4-oxo-tetrahydro-thiopyran-3-yl]-carbamic acid tert-butyl ester (10.3 g, 18.7 mmol) in anhydrous THF (400 mL) is added under argon LiAlH₄ (0.73 g, 18.7 mmol) in portions at −60° C. The reaction mixture is stirred for 3 h at −60° C. and then sequentially quenched with H₂O (0.73 mL), 4N NaOH (0.73 mL) and H₂O (2.2 mL). After stirring for 1 h the white precipitate is filtered off over Celite and the filtrate is evaporated. After repeated crystallization from THF-diisopropylether the title compound is obtained as the pure (3R*,4S*,5S*)-diastereoisomer: TLC (toluene-EtOAc 4:1) Rf=0.30; HPLC Rt_{A}=2.37 min; ESIMS [M+H+NH₃]⁺=564; ¹H-NMR (400 MHz, CDCl₃): δ 7.38 (m, 5H), 6.81 (m, 2H), 6.0 (s, 1H), 5.19 (s, 2H), 4.63 (d, 1H), 3.72 (m, 1H), 3.26 (dd, 1H), 3.04 (m, 1H), 2.84 (s, 1H), 2.75 (dt, 1H), 2.56 t, 2H), 2.48 (dd, 1H), 2.38 (m, 2H), 2.27 (dd, 1H), 2.05 (m, 1H), 1.48 (m, 2H), 1.43 (s, 9H), 1.33 (m, 2H), 0.88 (t, 3H).

### j) [(3R*,4S*,5S*)-5-(4-Benzyloxycarbonylamino-3-butyl-5-fluoro-benzyl)-4-hydroxy-1,1-dioxo-hexahydro-1lambda*6*-thiopyran-3-yl]-carbamic acid tert-butyl ester

The title compound is prepared in analogous manner as described for example 1f), starting from [(3R*,4S*,5S*)-5-(4-benzyloxycarbonylamino-3-butyl-5-fluoro-benzyl)-4-hydroxy-tetra-hydro-thiopyran-3-yl]-carbamic acid tert-butyl ester and oxone: TLC (toluene-EtOAc 2:1) Rf=0.25; HPLC Rt_{A}=2.13 min; ESIMS [M+H+NH₃]⁺=596; ¹H-NMR (400 MHz, DMSO-d₆): δ 8.94 (s, 1H), 7.38 (m, 5H), 6.90 (m, 3H), 5.19 (d, 1H), 5.11 (s, 2H), 3.71 (m, 1H), 3.0-3-3 (m, 5H), 2.96 (dd, 1H), 2.69 (m, 1H), 2.50 (m, 2H), 2.06 (m, 1H), 1.42 (m, 2H), 1.39 (s, 9H), 1.25 (m, 2H), 0.86 (t, 3H).

### k) [4-((3S*,4S*,5R*)-5-Amino-4-hydroxy-1,1-dioxo-hexahydro-1lambda*6*-thiopyran-3-ylmethyl)-2-butyl-6-fluoro-phenyl]-carbamic acid benzyl ester hydrochloride

The title compound is prepared in analogous manner as described for example 1g), starting from [(3R*,4S*,5S*)-5-(4-benzyloxycarbonylamino-3-butyl-5-fluoro-benzyl)-4-hydroxy-1,1-dioxo-hexahydro-1lambda*6*-thiopyran-3-yl]-carbamic acid tert-butyl ester and 4N HCl in dioxane: HPLC Rt_{A}=1.70 min; ESIMS [M+H]⁺=479; ¹H-NMR (400 MHz, CD₃OD): δ 7.38 (m, 5H), 6.90 (m, 2H), 5.19 (d, 2H), 3.4-3.6 (m, 5H), 3.09 (dd, 1H), 2.87 (m, 1H), 2.56 (t, 2H), 2.52 (m, 1H), 2.32 (m, 1H), 1.50 (m, 2H), 1.32 (m, 2H), 0.88 (t, 3H).

### l) {2-Butyl-4-[(3S*,4S*,5R*)-5-(3-tert-butyl-benzylamino)-4-hydroxy-1,1-dioxo-hexa-hydro-1lambda*6*-thiopyran-3-ylmethyl]-6-fluoro-phenyl}-carbamic acid benzyl ester

The title compound is prepared in analogous manner as described for example 1h), starting from [4-((3S*,4S*,5R*)-5-amino-4-hydroxy-1,1-dioxo-hexahydro-1lambda*6*-thiopyran-3-ylmethyl)-2-butyl-6-fluoro-phenyl]-carbamic acid benzyl ester hydrochloride and 3-tert-butyl-benzaldehyde: TLC (hexane-EtOAc 1:3) Rf=0.34; HPLC Rt_{A}=2.15 min; ESIMS [M+H]⁺=625; ¹H-NMR (400 MHz, CDCl₃): δ 7.2-7.4 (m, 8H), 7.13 (d, 1H), 6.90 (m, 2H), 6.0 (s, 1H), 5.19 (d, 2H), 4.06 (s, 1H), 3.90 (d, 1H), 3.76 (dm, 2H), 3.65 (m, 1H), 3.39 (dt, 1H), 3.12 (m, 2H), 3.06 (dd, 1H), 2.92 (dt, 1H), 2.5-2.7 (m, 5H), 2.39 (m, 1H), 1.48 (m, 2H), 1.36 (s, 9H), 1.32 (m, 2H), 0.87 (t, 3H).

### m) (3S*,4S*,5R*)-3-(4-Amino-3-butyl-5-fluoro-benzyl)-5-(3-tert-butyl-benzylamino)-1,1-dioxo-hexahydro-1lambda*6*-thiopyran-4-ol

The title compound is prepared in analogous manner as described for example 14b), starting from {2-butyl-4-[(3S*,4S*,5R*)-5-(3-tert-butyl-benzylamino)-4-hydroxy-1,1-dioxo-hexahydro-1lambda*6*-thiopyran-3-ylmethyl]-6-fluoro-phenyl}-carbamic acid benzyl ester: HPLC Rt_{A}=1.84 min; ESIMS [M+H]⁺=491; ¹H-NMR (600 MHz, DMSO-d₆): δ 7.35 (s, 1H), 7.24 (m, 2H), 7.13 (d, 1H), 6.67 (dd, 1H), 6.56 (s, 1H), 5.15 (d, 1H), 4.67 (s, 2H), 3.80 (dd, 1H), 3.70 (dd, 1H), 3.66 (dd, 1H), 3.63 (m, 1H), 3.3-3.5 (m, 2H), 3.15 (m, 1H), 2.97 (dd, 1H), 2.93 (dd, 1H), 2.77 (m, 1H), 2.60 (m, 1H), 2.45 (m, 1H), 2.29 (m,1H), 1.96 (m, 1H), 1.45 (m, 2H), 1.31 (m, 2H), 1.27 (s, 9H), 0.87 (t, 3H).

### Example 18

### 3S*,4S*,5R*)-3-(4-Amino-3-fluoro-5-pentyl-benzyl)-5-(3-tert-butyl-benzyl-amino)-1,1-dioxo-hexahydro-1lambda*6*-thiopyran-4-ol hydrochloride

The title compound is prepared in analogous manner as described for example 17, starting from 4-amino-3-fluoro-5-iodo-benzoic acid methyl ester and pent-1-yne: HPLC Rt_{A}=1.94 min; ESIMS [M+H]⁺=505.

### Example 19

### (3S*,4S*,5R*)-3-(4-Amino-3-butyl-5-fluoro-benzyl)-5-(3-tert-butyl-benzyl-amino)-1-oxo-tetrahydro-thiopyran-4-ol hydrochloride

### a) [(3R*,4S*,5S*)-5-(4-Benzyloxycarbonylamino-3-butyl-5-fluoro-benzyl)-4-hydroxy-1-oxo-hexahydro-1lambda*4*-thiopyran-3-yl]-carbamic acid tert-butyl ester

To a solution of [(3R*,4S*,5S*)-5-(4-benzyloxycarbonylamino-3-butyl-5-fluoro-benzyl)-4-hydroxy-tetrahydro-thiopyran-3-yl]-carbamic acid tert-butyl ester (example 17i)) (3.39 g, 6.2 mmol) in water-THF 1:1 (250 mL) is added oxone (3.89 g, 6.2 mmol) at 0-5° C. in small portions over a period of 4 h. After completion sodium meta-bisulfite is added and the product extracted with EtOAc. Combined extracts are washed with brine, dried over MgSO₄ and evaporated. The product obtained as a crystalline white solid suitable for use in the next step: TLC (CH₂Cl₂/MeOH 19:1) Rf=0.21; HPLC Rt_{A}=1.98 min; ESIMS [M+H+NH₃]⁺=580; ¹H-NMR (600 MHz, DMSO-d₆): δ 8.89 (s, 1H), 7.35 (m, 5H), 6.94 (d, 1H), 6.88 (m, 2H), 5.12 (s, 2H), 5.06 (d, 1H), 3.42 (m, 1H), 3.26 (m, 1H), 3.05 (m, 3H), 2.56 (m, 1H), 2.51 (m, 2H), 2.26 (t, 1H), 1.79 (m, 1H), 1.43 (m, 2H), 1,36 (s, 9H), 1.25 (m, 2H), 0.84 (t, 3H).

### b) [4-((3S*,4S*,5R*)-5-Amino-4-hydroxy-1-oxo-hexahydro-1lambda*4*-thiopyran-3-ylmethyl)-2-butyl-6-fluoro-phenyl]-carbamic acid benzyl ester hydrochloride

A solution of [(3R*,4S*,5S*)-5-(4-benzyloxycarbonylamino-3-butyl-5-fluoro-benzyl )-4-hydroxy-1-oxo-hexahydro-1lambda*4*-thiopyran-3-yl]-carbamic acid tert-butyl ester (0.45 g, 0.8 mmol) in 5N HCl in iPrOH (7 mL) is stirred for 2 h at 25° C. The solvent is evaporated and the residue dried to yield to title compound as a yellow foam suitable for use in the next step: TLC (CH₂Cl₂-MeOH—AcOH—H₂O 180:20:2:1) Rf=0.08; HPLC Rt_{A}=1.63 min; ESIMS [M+H]⁺=463.

### c) {2-Butyl-4-[(3S*,4S*,5R*)-5-(3-tert-butyl-benzylamino)-4-hydroxy-1-oxo-tetrahydro-thiopyran-3-ylmethyl]-6-fluoro-phenyl}-carbamic acid benzyl ester

The title compound is prepared in analogous manner as described for example 1h), starting from [4-((3S*,4S*,5R*)-5-amino-4-hydroxy-1-oxo-hexahydro-1lambda*4*-thiopyran-3-ylmethyl)-2-butyl-6-fluoro-phenyl]-carbamic acid benzyl ester hydrochloride and 3-tert-butyl-benzaldehyde: TLC (CH₂Cl₂-MeOH 19:1) Rf=0.31; HPLC Rt_{A}=2.04 min; ESIMS [M+H]⁺=609; ¹H-NMR (600 MHz, DMSO-d₆+TFA): δ 9.21 (s, 1H), 8.94 (s, 1H), 8.87 (s, 1H), 7.56 (s, 1H), 7.44 (d, 1H), 7.35 (m, 7H), 6.92 (d, 1H), 6.88 (s, 1H), 5.10 (s, 2H), 4.21 (m, 1H), 3.52 (m, 3H), 3.14 (d, 1H), 2.86 (t, 1H), 2.78 (m, 1H), 2.5 (m, 4H), 1.42 (m, 2H), 1.28 (s, 9H), 1.25 (m, 2H), 0.82 (t, 3H).

### d) (3S*,4S*,5R*)-3-(4-Amino-3-butyl-5-fluoro-benzyl)-5-(3-tert-butyl-benzylamino)-1-oxo-tetrahydro-thiopyran-4-ol hydrochloride

The title compound is prepared in analogous manner as described for example 14b), starting from {2-butyl-4-[(3S*,4S*,5R*)-5-(3-tert-butyl-benzylamino)-4-hydroxy-1-oxo-tetrahydro-thiopyran-3-ylmethyl]-6-fluoro-phenyl}-carbamic acid benzyl ester: TLC (CH₂Cl₂-MeOH 19:1) Rf=0.27; HPLC Rt_{A}=1.65 min; ESIMS [M+H]⁺=475; ¹H-NMR (600 MHz, DMSO-d₆): δ 9.67 (s, 1H), 9.01 (s, 1H), 7.64 (s, 1H), 7.43 (dt, 1H), 7.35 (m, 2H), 6.82 (d, 1H), 6.71 (s, 1H), 4.23 (m, 1H), 4.18 (m, 1H), 3.56 (m, 3H), 3.06 (d, 1H), 2.91 (t, 1H), 2.74 (d, 1H), 2.4-2.6 (m, 4H), 1.48 (m, 2H), 1.33 (m, 2H), 1.29 (s, 9H), 0.89 (t, 3H).

### Example 20

### (3S*,4S*,5R*)-3-(4-Amino-3-butyl-5-fluoro-benzyl)-5-(3-tert-butyl-benzyl-amino)-1-imino-1-oxo-hexahydro-1lambda*6*-thiopyran-4-ol hydrochloride

### a) [(3R*,4S*,5S*)-5-(4-Benzyloxycarbonylamino-3-butyl-5-fluoro-benzyl)-4-hydroxy-1-imino-1-oxo-hexahydro-1lambda*6*-thiopyran-3-yl]-carbamic acid tert-butyl ester

To a solution of [(3R*,4S*,5S*)-5-(4-benzyloxycarbonylamino-3-butyl-5-fluoro-benzyl)-4-hydroxy-1-oxo-hexahydro-1lambda*4*-thiopyran-3-yl]-carbamic acid tert-butyl ester (example 19a)) (1.0 g, 1.7 mmol) in CH₂Cl₂ (20 mL) is added under argon trifluoroacetamide (0.4 g, 3.4 mmol), MgO (0.28 g, 7 mmol) and Rh₂(O₂CCH₃)₄ (0.032 g, 0.07 mmol) and the mixture is stirred for 16 h at 25° C. The mixture is filtered over Celite and evaporated, taken up in MeOH and after addition of 5 equivalents of K₂CO₃ stirred for 2 h at 25° C. The crude product is obtained by extraction with EtOAc. Combined extracts are washed with brine, dried over MgSO₄ and evaporated. The title compound is obtained after purification by flash-chromato-graphy on silica gel (hexane-EtOAc 2:1 to EtOAc) as a beige solid: TLC (EtOAc) Rf=0.38; HPLC Rt_{A}=1.95 min; ESIMS [M+H]⁺=578; ¹H-NMR (600 MHz, DMSO-d₆): δ 8.92 (s, 1H), 7.3 (m, 6H), 6.92 (d, 1H), 6.87 (s, 1H), 6.76 (d, 1H), 5.11 (s, 2H), 5.06 (d, 1H), 3.74 (m, 1H), 3.13 (m, 1H), 3.05 (m, 2H), 2.93 (t, 1H), 2.77 (t, 1H), 2.69 (m, 1H), 2.5 (m, 2H), 2.42 (m, 1H), 2.10 (m, 1H), 1.43 (m, 2H), 1,38 (s, 9H), 1.25 (m, 2H), 0.84 (t, 3H).

### b) [4-((3S*,4S*,5R*)-5-Amino-4-hydroxy-1-imino-1-oxo-hexahydro-1lambda*6*-thio-pyran-3-yl methyl)-2-butyl-6-fluoro-phenyl]-carbamic acid benzyl ester

The title compound is prepared in analogous manner as described for example 1g), starting from [(3R*,4S*,5S*)-5-(4-benzyloxycarbonylamino-3-butyl-5-fluoro-benzyl)-4-hydroxy-1-imino-1-oxo-hexahydro-1lambda*6*-thiopyran-3-yl]-carbamic acid tert-butyl ester and 4N HCl in dioxane: TLC (CH₂Cl₂-MeOH—AcOH—H₂O 180:20:2:1) Rf=0.13; HPLC Rt_{A}=1.63 min; ESIMS [M+H]⁺=478.

### c) {2-Butyl-4-[(3S*,4S*,5R*)-5-(3-tert-butyl-benzylamino)-4-hydroxy-1-imino-1-oxo-hexa-hydro-1lambda*6*-thiopyran-3-ylmethyl]-6-fluoro-phenyl}-carbamic acid benzyl ester

The title compound is prepared in analogous manner as described for example 1 h), starting from [4-((3S*,4S*,5R*)-5-amino-4-hydroxy--imino-1-oxo-hexahydro-1lambda*6*-thiopyran-3-ylmethyl)-2-butyl-6-fluoro-phenyl]-carbamic acid benzyl ester and 3-tert-butyl-benzaldehyde: TLC (CH₂Cl₂-MeOH 19:1) Rf=0.38; HPLC Rt_{A}=2.06 min; ESIMS [M+H]⁺=624.

### d) (3S*,4S*,5R*)-3-(4-Amino-3-butyl-5-fluoro-benzyl)-5-(3-tert-butyl-benzylamino)-1-imino-1-oxo-hexahydro-1lambda*6*-thiopyran-4-ol hydrochloride

The title compound is prepared in analogous manner as described for example 14b), starting from {2-butyl-4-[(3S*,4S*,5R*)-5-(3-tert-butyl-benzylamino)-4-hydroxy-1-imino-1-oxo-hexa-hydro-1lambda*6*-thiopyran-3-ylmethyl]-6-fluoro-phenyl}-carbamic acid benzyl ester: TLC (CH₂Cl₂-MeOH 19:1) Rf=0.32; HPLC Rt_{A}=1.70 min; ESIMS [M+H]⁺=490; ¹H-NMR (600 MHz, DMSO-d₆): δ 10.0 (s, 1H), 9.22 (s, 1H), 7.71 (s, 1H), 7.43 (d, 1H), 7.41 (d, 1H), 7.35 (d, 1H), 6.89 (d, 1H), 6.76 (s, 1H), 4.35 (s, 1H), 4.28 (m, 1H), 4.20 (m, 1H), 3.91 (m, 1H), 3.77 (t, 1H), 3.47 (m, 1H), 3.36 (m, 1H), 3.25 (m,1H), 3.08 (dd, 1H), 2.56 (m, 3H), 2.39 (dd, 1H), 2.09 (m, 1H), 1.50 (m, 2H), 1.33 (m, 2H), 1.29 (s, 9H), 0.89 (t, 3H).

### Example 21

### (3S*,4S*,5R*)-3-(4-Amino-3-butyl-5-chloro-benzyl)-5-(3-tert-butyl-benzyl-amino)-1,1-dioxo-hexahydro-1lambda*6*-thiopyran-4-ol hydrochloride

### a) 4-Amino-3-butyl-benzoic acid methyl ester

The title compound is prepared in analogous manner as described for examples 17b) and 17c), starting from 4-amino-3-iodo-benzoic acid methyl ester and but-1-yne: TLC (hexane-EtOAc 1:1) Rf=0.63; HPLC Rt_{A}=1.78 min; ESIMS [M+H]⁺=208.

### b) 4-Amino-3-butyl-5-chloro-benzoic acid methyl ester

To a solution of 4-amino-3-butyl-benzoic acid methyl ester (4.2 g, 19.8 mmol) in ACN (100 mL) is added N-chlorosuccinimide (2.8 g, 19.8 mmol), and the mixture is heated at reflux for 1 h. After evaporation the residue is purified by flash-chromatography on silica gel (hexane-EtOAc 4:1) as a beige solid: TLC (hexane-EtOAc 3:1) Rf=0.49; HPLC Rt_{A}=2.15 min; ESIMS [M+H]⁺=242 and 244.

### c) (3S*,4S*,5R*)-3-(4-Amino-3-butyl-5-chloro-benzyl)-5-(3-tert-butyl-benzylamino)-1,1-dioxo-hexahydro-1lambda*6*-thiopyran-4-ol hydrochloride

The title compound is prepared in analogous manner as described for examples 17d) to 17m), starting from 4-amino-3-butyl-5-chloro-benzoic acid methyl ester: TLC (CH₂Cl₂-MeOH 19:1) Rf=0.62; HPLC Rt_{A}=1.99 min; ESIMS [M+H]⁺=507 and 509; ¹H-NMR (600 MHz, DMSO-d₆): δ 9.94 (s, 1H), 9.06 (s, 1H), 7.66 (s, 1H), 7.43 (d, 1H), 7.37 (m, 2H), 6.93 (s, 1H), 6.74 (s, 1H), 6.1 (s, 1H), 4.22 (m, 2H), 3.86 (dt, 1H), 3.61 (m, 3H), 3.16 (m, 2H), 2.98 (d, 1H), 2.75 (dt, 1H), 2.48 (t, 2H), 2.32 (dd, 1H), 1.99 (m, 1H), 1.47 (m, 2H), 1.31 (m, 2H), 1.29 (s, 9H), 0.88 (t, 3H).

### Examples 21a -21h

The compounds listed in Table 3 can be prepared by a procedure analogous to that used in example 17.

TABLE 3

| | | HPLC | |
|---|---|---|---|
| | | Rt [min] | |
| Example | Compound | Method | ESIMS |
| 21a | (3S*,4S*,5R*)-3-(4-Amino-3-butyl-5-fluoro-benzyl)- | 1.82 A | [M − H]⁺ = 507 |
| | 5-(3-tert-butyl-2-hydroxy-benzylamino)-1,1-dioxo- | | |
| | hexahydro-1lambda*6*-thiopyran-4-ol | | |
| 21b | (3S*,4S*,5R*)-3-(4-Amino-3-butyl-5-fluoro-benzyl)- | 1.88 A | [M − H]⁺ = 509 |
| | 5-(3-tert-butyl-5-fluoro-benzylamino)-1,1-dioxo- | | |
| | hexahydro1lambda*6*-thiopyran-4-ol | | |
| 21c | (3S*,4S*,5R*)-3-(4-Amino-3-butyl-5-fluoro-benzyl)- | 4.33 D | [M − H]⁺ = 505 |
| | 5-[3-(3-methyl-oxetan-3-yl)-benzylamino]-1,1- | | |
| | dioxo-hexahydro-1lambda*6*-thiopyran-4-ol | | |
| 21d | (3S*,4S*,5R*)-3-(4-Amino-3-butyl-5-fluoro-benzyl)- | 1.51 A | [M − H]⁺ = 501 |
| | 1,1-dioxo-5-(3-pyrazol-1-yl-benzylamino)-hexa- | | |
| | hydro-1lambda*6*-thiopyran-4-ol | | |
| 21e | (3S*,4S*,5R*)-3-(4-Amino-3-butyl-5-fluoro-benzyl)- | 1.58 A | [M − H]⁺ = 495 |
| | 5-{[1-(2,2-dimethyl-propyl)-1H-pyrazol-4-ylmethyl]- | | |
| | amino}-1,1-dioxo-hexahydro1lambda*6*-thio- | | |
| | pyran-4-ol | | |
| 21f | (3S*,4S*,5R*)-3-(4-Amino-3-butyl-5-fluoro-benzyl)- | 6.47 D | [M − H]⁺ = 496 |
| | 5-{[5-(2,2-dimethyl-propyl)-isoxazol-3-ylmethyl]- | | |
| | amino}-1,1-dioxo-hexahydro-1lambda*6*-thio- | | |
| | pyran-4-ol | | |
| 21g | (3S*,4S*,5R*)-3-(4-Amino-3-butyl-5-fluoro-benzyl)- | 5.96 D | [M − H]⁺ = 495 |
| | 5-{[5-(2,2-dimethyl-propyl)-1H-pyrazol-3-ylmethyl]- | | |
| | amino}-1,1-dioxo-hexahydro-1lambda*6*-thio- | | |
| | pyran-4-ol | | |
| 21h | (3S*,4S*,5R*)-3-(4-Amino-3-butyl-5-fluoro-benzyl)- | 1.41 A | [M − H]⁺ = 493 |
| | 5-[3-(1-hydroxy-1-methyl-ethyl)-benzylamino]-1,1- | | |
| | dioxo-hexahydro-1lambda*6*-thiopyran-4-ol | | |

### 3-tert-Butyl-5-fluoro-benzaldehyde (Example 21b)

To a solution of 1-bromo-3-tert-butyl-5-fluoro-benzene (1.04 g, 4.5 mmol) in anhydrous THF (25 mL) is added under argon a 2.5M solution of nBuLi in hexane (1.9 mL, 4.7 mmol) at −78° C. After stirring for 0.5 h at −78° C. DMF (0.7 mL, 9 mmol) is slowly added by syringe, and the mixture is stirred again for 1.5 h at −78° C. The reaction mixture is added to 0.5N HCl and extracted with Et₂O. The product obtained as a light yellow solid is suitable for use in the next step: TLC (hexane-EtOAc 1:1) Rf=0.36; HPLC Rt_{A}=2.08 min; ¹H-NMR (400 MHz, CDCl₃): δ 9.95 (s, 1H), 7.69 (m, 1H), 7.36 (m, 2H), 1.38 (s, 9H).

### 3-(3-Methyl-oxetan-3-yl)-benzaldehyde (Example 21c)

### a) 2-(3-Bromo-phenyl)-2-methyl-malonic acid dimethyl ester

A solution of 2-(3-bromo-phenyl)-malonic acid dimethyl ester (5 g, 15.9 mmol) in EtOH (100 mL) is cooled at 0° C. and is treated in portions with sodium (0.46 mg, 19 mmol). The mixture is stirred for 15 min at 0° C., then methyl iodide (1.09 mL, 17.5 mmol) is added. The mixture is stirred for 4 h at 25° C. After quenching with water the mixture is concentrated, the residue is extracted with EtOAc, and the extract is washed with water and brine, dried over Na₂SO₄, filtered and evaporated. The title compound is obtained after flash-chromatography on silica gel (cyclohexane-EtOAc 4:1) as a light yellow syrup: ESIMS [M+H]⁺=329, 331; ¹H-NMR (400 MHz, CDCl₃): δ 7.52 (t, 1H), 7.42 (m, 1H), 7.31 (m, 1H), 7.20 (m, 1H), 4.22 (dd, 4H), 1.84 (s, 3H), 1.25 (t, 6H).

### b) 2-(3-Bromo-phenyl)-2-methyl-propane-1,3-diol

A solution of 2-(3-bromo-phenyl)-2-methyl-malonic acid dimethyl ester (3.86 g, 11.7 mmol) in Et₂O (40 mL) is added to a solution of LiAlH₄ (0.67 g, 17.6 mmol) in Et₂O (60 mL) at 0° C. The reaction mixture is heated at 40° C. for 4 h. After cooling at 0° C., the reaction mixture is quenched with saturated NaHCO₃ solution and concentrated. The title compound is obtained after flash-chromatography on silica gel (cyclohexane-EtOAc 1:1) as a colorless syrup: ESIMS [M+H₂O]⁺=262, 264; ¹H-NMR (400 MHz, CDCl₃): δ 7.57 (t, 1H), 7.40 (m, 2H), 7.23 (d, 1H), 3.95 (dd, 2H), 3.83 (dd, 2H), 1.27 (s, 3H).

### c) 3-(3-Bromo-phenyl)-3-methyl-oxetane

A solution of 2-(3-bromo-phenyl)-2-methyl-propane-1,3-diol (1 g, 4.08 mmol) in toluene (40 mL) is treated with PPh₃ (2.14 g, 8.16 mmol), then with zinc N,N-dimethyl dithiocarbonate (1.93 g, 6.12 mmol) and DEAD (1.32 mL, 8.16 mmol). The reaction mixture is stirred for 15 h at 25° C. and then evaporated. The title compound is obtained after flash-chromatography on silica gel (cyclohexane-EtOAc 1:1) as a colorless syrup: ESIMS [M+H₂O]⁺=243, 245; ¹H-NMR (400 MHz, CDCl₃): δ 7.37 (dt, 1H), 7.33 (t, 1H), 7.22 (t, 1H), 7.12 (dt, 1H), 4.92 (d, 2H), 4.62 (d, 2H), 1.70 (s, 3H).

### d) 3-(3-Methyl-oxetan-3-yl)-benzaldehyde

A solution of 3-(3-bromo-phenyl)-3-methyl-oxetane (0.3 g, 1.32 mmol) in THF (10 mL) cooled at −78° C. is treated dropwise with a 1.6 M solution of nBuLi in THF (0.95 mL, 1.52 mmol). The mixture is stirred for 1 h at −78° C., then DMF (0.5 mL, 6.61 mmol) is added, and the mixture is stirred for 1 h at 25° C. The mixture is quenched with saturated NH₄Cl and extracted with EtOAc, and the extract is washed with brine, dried over Na₂SO₄, filtered and evaporated. The title compound is obtained after flash-chromatography on silica gel (cyclohexane-EtOAc 1:1) as a colorless syrup: ESIMS [M+H₂O]⁺=194; ¹H-NMR (400 MHz, CDCl₃): δ 10.00 (s, 1H), 7.74 (dt, 1H), 7.72 (s, 1H), 7.53 (t, 1H), 7.47 (dt, 1H), 4.96 (d, 2H), 4.67 (d, 2H), 1.75 (s, 3H).

### 5-(2,2-Dimethyl-propyl)-isoxazole-3-carbaldehyde (Example 21f)

### a) (Z)-2-Hydroxy-6,6-dimethyl-4-oxo-hept-2-enoic acid ethyl ester

To an ice-cooled solution of sodium ethanolate (128.5 g, 1.79 mol) in EtOH (2.5 L) under nitrogen atmosphere is added 4,4-dimethyl-pentan-2-one (195.0 g, 1.71 mol). Half an hour later, oxalic acid diethyl ester (231.5 g, 1.71 mol) is added. After being stirred at 25° C. for 24 h, the reaction mixture is diluted with water, and acidified to pH 2 with 6N aqueous hydrochloric acid. The mixture is contracted to about 1 L and extracted with CH₂Cl₂. The combined extracts are washed with brine, dried over Na₂SO₄, and evaporated to yield the product as a brown liquid: ESIMS [M+H]⁺=215; ¹H-NMR (300 MHz, CDCl₃): δ 6.32 (s, 1H), 4.35 (q, 2H), 2.33 (s, 2H), 1.60 (t, 3H), 1.04 (s, 9H).

### b) 5-(2,2-Dimethyl-propyl)-isoxazole-3-carboxylic acid

To a solution of (Z)-2-hydroxy-6,6-dimethyl-4-oxo-hept-2-enoic acid ethyl ester (298.5 g, 1.39 mol) in EtOH (1600 ml) is added hydroxylamine hydrochloride (106.5 g, 1.53 mol), and the resulting solution is stirred at room temperature for 24 h. 2N NaOH (1740 ml, 3.48 mol) is added to the reaction mixture, and the resulting solution is stirred at 25° C. for 2 h. The reaction mixture is acidified with 6N HCl, concentrated to about 3 L, and extracted with EtOAc. The combined organic layers are washed with brine, dried over MgSO₄ and evaporated. The resulting solid is washed with ether and dried to afford the title compound: ¹H-NMR (300 MHz, DMSO-d₆): δ 6.61 (s, 1H), 2.72 (s, 2H), 0.94 (s, 9H).

### c) 5-(2,2-Dimethyl-propyl)-isoxazole-3-carboxylic acid tert-butylamide

To a solution of 5-(2,2-dimethyl-propyl)-isoxazole-3-carboxylic acid (125.4 g, 0.685 mol) in THF (1500 ml) and ACN (1500 ml) are added HOBT (101.75 g, 0.753 mol) and EDC (144.3 g, 0.753 mol). Then tert-butyl amine (86.7 ml, 0.821 mol) is added dropwise under nitrogen, and the mixture is stirred at 25° C. for 1.5 h. The solvents are evaporated and the residue is taken up in CH₂Cl₂. The mixture is washed with NaHCO₃ solution, dried over Na₂SO₄ and evaporated. The residue is purified by chromatography on silica gel (CH₂Cl₂) to give the product as colourless solid: ESIMS: [M+H]⁺=239.

### d) 5-(2,2-Dimethyl-propyl)-isoxazole-3-carbonitrile

A mixture of 5-(2,2-dimethyl-propyl)-isoxazole-3-carboxylic acid tert-butylamide (58.0 g, 0.243 mol) and phosphorus(III)oxychloride (156 ml, 1.70 mol) is heated under nitrogen at reflux for 2 h. The reaction mixture is cooled to 25° C. and concentrated to remove excess phosphorus(III)oxychloride. The residue is diluted with CH₂Cl₂ (2000 ml) and washed with saturated aqueous sodium bicarbonate (500 ml×2). The organic layer is washed with brine, dried over sodium sulfate, and concentrated. The residue is purified by chromatography on silica (CH₂Cl₂/hexanes 1/1) to yield the target compound as yellow liquid: ¹H-NMR (300 MHz, CDCl₃): δ 6.36 (s, 1H), 2.74 (s, 2H), 1.00 (s, 9H).

### e) 5-(2,2-Dimethyl-propyl)-isoxazole-3-carbaldehyde

To a solution of 5-(2,2-dimethyl-propyl)-isoxazole-3-carbonitrile (0.1 g, 0.6 mmol) in CH₂Cl₂ (16 mL) is added at −78° C. under argon a 1M solution of DIBAL (1.2 mL, 1.2 mmol) in hexane within 1 h. The reaction mixture is stirred for 4 h at −78° C., quenched at −78° C. with ice-water and stirred at room temperature for 1 h. The solution is partitioned between EtOAc and water. The organic phase is washed with brine, dried over Na₂SO₄ and evaporated. The residue is separated by flash-chromatography on silica gel (EtOAc-hexane 20:1 to 5:1) to afford the title compound as a colorless oil: TLC (hexane-EtOAc 9:1) Rf=0.38; ESIMS [M+H]⁺=168; ¹H-NMR (400 MHz, CDCl₃): δ 10.13 (s, 1H), 6.38 (s, 1H), 2.71 (s, 2H), 0.98 (s, 9H).

### Example 22

### (3S,4S,5R)-3-(4-Amino-benzyl)-5-[1-(3-tert-butyl-phenyl)-cyclopropyl-amino]-1,1-dioxo-hexahydro-1lambda*6*-thiopyran-4-ol hydrochloride

### a) 3-(4-Bromo-benzyl)-4-oxo-3,4-dihydro-2H-thiopyran-3-carboxylic acid allyl ester

To a solution of 4-oxo-3,4-dihydro-2H-thiopyran-3-carboxylic acid allyl ester (3.5 g, 17.4 mmol) in methyl-ethylketone (80 mL) is added 1-bromo-4-bromomethyl-benzene (5.8 g, 22.7 mmol) and K₂CO₃ (7.4 g, 52.4 mmol) and the reaction mixture is stirred for 6 h at 25° C. The reaction mixture is filtered and the filtrate is evaporated. The residual oil is dissolved in EtOAc, washed with saturated NaHCO₃ solution and brine, dried over MgSO₄ and evaporated to yield the title compound after crystallization from Et₂O-hexane as yellow crystals: TLC (hexane-EtOAc 3:1) Rf=0.35; ESIMS [M+H]⁺=367, 369; NMR (400 MHz, CDCl₃): δ 7.40 (m, 3H), 7.19 (d, 2H), 6.21 (d, 1H), 5.85 (m, 1H), 5.28 (dd, 2H), 4.63 (m, 2H), 3.55 (d, 1H), 3.36 (d, 1H), 3.22 (d, 1H), 3.03 (d, 1H).

### b) 3-(4-Bromo-benzyl)-2,3-dihydro-thiopyran-4-one

To a solution of 3-(4-bromo-benzyl)-4-oxo-3,4-dihydro-2H-thiopyran-3-carboxylic acid allyl ester (9.92 g, 27 mmol) in anhydrous THF (400 mL) is added under argon dimedone (23.0 g, 162 mmol) and Pd(PPh₃)₄ (0.94 g, 0.81 mmol). The reaction mixture is stirred for 2 h at 25° C. and then evaporated. The product is obtained after purification by flash-chromatography on silica gel (toluene-EtOAc 6:1 to 4:1) and crystallization from hexane-Et₂O as a light yellow solid: TLC (toluene-EtOAc 3:1) Rf=0.56; ESIMS [M+H]⁺=283 and 285; ¹H-NMR (400 MHz, CDCl₃): δ 7.47 (m, 3H), 7.14 (d, 1H), 6.21 (d, 1H), 3.2 (m, 2H), 2.88 (m, 1H), 2.79 (m, 2H).

### c) (3S*,4R*)-3-(4-Bromo-benzyl)-3,4-di hydro-2H-thiopyran-4-ol

To a solution of 3-(4-bromo-benzyl)-2,3-dihydro-thiopyran-4-one (6.9 g, 24 mmol) in THF-MeOH 1:1 (200 mL) is added CeCl₃ (11.95 g, 48 mmol) and after 0.5 h stirring at 25° C. the NaBH₄ (1.83 g, 48 mmol) in small portions over a period of 1.5 h. After stirring for 1 h the reaction mixture is diluted with saturated NH₄Cl solution and the organic solvents are removed under reduced pressure. The product is extracted from the aqueous phase with EtOAc. The combined extracts are washed with water and brine, dried over MgSO₄ and evaporated to yield the title compound as a beige foam: TLC (hexane-EtOAc 1:1) Rf=0.52; ESIMS [M−H₂O]⁺=267, 269; ¹H-NMR (400 MHz, CDCl₃): δ 7.44 (d, 2H), 7.15 (d, 2H), 6.32 (d, 1H), 5.93 (dd, 1H), 4.5 (m, 1H), 4.03 (m, 1H), 2.92 (dd, 1H), 2.83 (d, 1H), 2.69 (dd, 1H), 2.54 (d, 1H), 2.08 (m, 1H).

### d) (3S*,4R*)-3-(4-Bromo-benzyl)-1,1-dioxo-1,2,3,4-tetrahydro-1lambda*6*-thiopyran-4-ol

To a solution of (3S*,4R*)-3-(4-bromo-benzyl)-3,4-dihydro-2H-thiopyran-4-ol (10.4 g, 36.6 mmol) in THF (225 mL) are added water (225 mL) and in portions the oxone (50.5 g, 80.5 mmol) at 25-30° C. The reaction mixture is stirred over night at room temperature. After addition of NaOAc (12.5 g, 146 mmol) the excess oxone is destroyed with NaS₂O₃ (10 g). The reaction mixture is diluted with water and the product is extracted with EtOAc. The combined extracts are washed with water and brine, dried over MgSO₄ and evaporated. The title compound is obtained in pure form after crystallization from Et₂O as a white solid: TLC (hexane-EtOAc 1:1) Rf=0.15; ESIMS [M+H+NH₃]⁺=334, 346; ¹H-NMR (400 MHz, CDCl₃): δ 7.51 (d, 2H), 7.18 (d, 2H), 6.45 (s, 2H), 4.05 (m, 1H), 3.41 (dd, 1H), 3.02 (dd, 1H), 2.96 (m, 1H), 2.78 (m, 3H)

### e) 1-tert-Butyl-3-(1-isocyanato-cyclopropyl)-benzene

To a solution of bis-(trichloromethyl)-carbonate (4.05 g, 13.5 mmol) in CH₂Cl₂ (100 mL) is slowly added under argon at 0-5° C. a solution of 1-(3-tert-butyl-phenyl)-cyclopropylamine (2.55 g, 13.5 mmol) and DIPEA (5.86 mL, 13.5 mmol) in CH₂Cl₂ (100 mL) over a period of 1 h. After stirring for 1 h at 0° C. the reaction mixture is washed with cold NaHCO₃ solution, dried over MgSO₄ and evaporated. The crude product, a dark yellow oil, is directly used for the next step: TLC (hexane-EtOAc 3:1) Rf=0.70; ESIMS [M+H+NH₃]⁺=233.

### f) (3aR*,7S*,7aR*)-7-(4-Bromo-benzyl)-3-[1-(3-tert-butyl-phenyl)-cyclopropyl]-5,5-dioxo-hexahydro-1-oxa-5lambda*6*-thia-3-aza-inden-2-one

To a suspension of 1-tert-butyl-3-(1-isocyanato-cyclopropyl)-benzene (2.62 g, 12.2 mmol) and (3S*,4R*)-3-(4-bromo-benzyl)-1,1-dioxo-1,2,3,4-tetrahydro-1lambda*6*-thiopyran-4-ol (3.17 g, 10 mmol) in ACN (20 mL) is added DBU (0.19 mL, 1.2 mmol) and the resulting solution is stirred for 16 h at 50° C. After cooling the crystallized product is filtered off, washed with cold ACN-Et₂O 1:1 and dried to provide the title product as white crystals: TLC (hexane-EtOAc 1:1) Rf=0.67; ESIMS [M+H+NH₃]⁺=549, 551; ¹H-NMR (400 MHz, DMSO-d₆): δ 7.56 (d, 2H), 7.43 (s, 1H), 7.2-7.3 (m, 5H), 4.38 (m, 1H), 4.26 (m, 1H), 2.6-3.3 (m, 7H), 1.43 (m, 2H), 1.24 (s, 9H), 1.05 (m, 2H).

### g) (3S*,4R*,5R*)-3-(4-Bromo-benzyl)-5-[-(3-tert-butyl-phenyl)-cyclopropylamino]-1,1-dioxo-hexahydro-1lambda*6*-thiopyran-4-ol

The title compound is prepared in analogous manner as described for example 51 g) of WO-2007/093621, starting from (3aR*,7S*,7a R*)-7-(4-bromo-benzyl)-3-[-(3-tert-butyl-phenyl)-cyclopropyl]-5,5-dioxo-hexahydro-1-oxa-5lambda*6*-thia-3-aza-inden-2-one and Ba(OH)₂×8H₂O: TLC (hexane-EtOAc 1:1) Rf=0.34; ESIMS [M+H]⁺=506, 508; ¹H-NMR (400 MHz, CDCl₃): δ 7.47 (d, 2H), 7.26 (m, 3H), 7.07 (d, 2H), 7.0 (d, 1H), 3.62 (s, 1H), 2.5-3.2 (m, 8H), 2.27 (m, 1H), 1.96 s, 1H), 1.33 (s, 9H), 0.95 (m, 4H). h) (3S*,5R*)-3-(4-Bromo-benzyl)-5-[-(3-tert-butyl-phenyl)-cyclopropylamino]-1,1-dioxo-tetrahydro-1lambda*6*-thiopyran-4-one

To a solution of oxalyl chloride (0.92 mL, 9.8 mmol) in CH₂Cl₂ is added under argon at −78° C. a solution of DMSO (1.08 mL, 14.9 mmol) in CH₂Cl₂ (5 mL). After 10 min stirring at −78° C. a solution of (3S*,4R*,5R*)-3-(4-Bromo-benzyl )-5-[1-(3-tert-butyl-phenyl)-cyclopropylamino]-1,1-dioxo-hexahydro-1lambda*6*-thiopyran-4-ol (1.72 g, 2.7 mmol) in CH₂Cl₂(10 mL) is added. After stirring for 45 min at −70° C. 1-ethylpiperidine (3.8 mL, 27 mmol) is added at −70° C. and the reaction mixture is stirred for another 0.5 h at −70° C. followed by 1 h at −40° C. The reaction mixture is poured onto cold citric acid solution, basified with NaOH and extracted with CH₂Cl₂. The combined organic layers are washed with 5% K₂CO₃ solution and water, dried over MgSO₄ and evaporated. The crude product is directly used as such for the next step: TLC (hexane-EtOAc 1:1) Rf=0.62; ESIMS [M+H]⁺=504, 506.

### i) (3S*,4S*,5R*)-3-(4-Bromo-benzyl)-5-[-(3-tert-butyl-phenyl)-cyclopropylamino]-1,1-dioxo-hexahydro-1lambda*6*-thiopyran-4-ol

To a solution of (3S*,5R*)-3-(4-bromo-benzyl)-5-[1-(3-tert-butyl-phenyl)-cyclopropylamino]-1,1-dioxo-tetrahydro-1lambda*6*-thiopyran-4-one (1.36 g, 2.65 mmol) in anhydrous THF is added at −60° C. under argon LiAlH₄ (0.033 g, 0.79 mmol) and the reaction mixture is stirred for 0.5 h at −40° C. After addition of water (0.04 mL), 4N NaOH (0.05 mL) and water (0.1 mL) the reaction mixture is stirred for 0.5 h, filtered over Celite and evaporated. The crude product is purified by flash-chromatography on silica gel (hexane-EtOAc 2:1 to 1:1) to yield as the minor product the (3S*,4R*,5R*)-diastereoisomer [TLC (hexane-EtOAc 1:1) Rf=0.34] and the title compound as the major product as a yellow oil: TLC (hexane-EtOAc 1:1) Rf=0.16; ESIMS [M+H]⁺=506, 508; ¹H-NMR (600 MHz, DMSO-d₆+TFA): δ 7.71 (s, 1H), 7.53 (d, 1H), 7.46 (d, 2H), 7.43 (d, 1H), 7.39 (t, 1H), 7.11 (d, 2H), 3.61 (m, 1H), 3.37 (t, 1H), 3.33 (t, 1H), 3.19 (t, 1H), 3.02 (m, 2H), 2.74 (m, 1H), 2.51 (m, 1H), 2.04 (m, 1H), 1.47 (m, 1H), 1.32 (m, 2H), 1.28 (s, 9H), 1.01 (m, 1H).

### j) (3S*,4S*,5R*)-3-(4-Azido-benzyl)-5-[-(3-tert-butyl-phenyl)-cyclopropylamino]-1,1-dioxo-hexahydro-1lambda*6*-thiopyran-4-ol

To a solution of (3S*,4S*,5R*)-3-(4-bromo-benzyl )-5-[1-(3-tert-butyl-phenyl)-cyclopropyl-amino]-1,1-dioxo-hexahydro-1lambda*6*-thiopyran-4-ol (0.88 g, 1.7 mmol), sodium ascorbate (0.034 g, 0.17 mmol) and CuI (0.066 g, 0.34 mmol) in EtOH—H₂O 7:3 (40 mL) is added (1S,2S)-N,N′-dimethyl-cyclohexane-1,2-diamine (0.074 g, 0.51 mmol) and NaN₃ (0.339 g, 5.1 mmol) and the reaction mixture is heated for 5 h at reflux under argon. After addition of EtOAc the organic layer is washed with water and brine, dried over MgSO₄ and evaporated. The crude product is purified by flash-chromatography on silica gel (hexane-EtOAc 3:1 to 1:2) to yield the title compound as a light yellow foam: TLC (hexane-EtOAc 1:1) Rf=0.17; ESIMS [M+H]⁺=469; ¹H-NMR (600 MHz, DMSO-d₆): δ 7.42 (s, 1H), 7.22 (m, 2H),7.19 (d, 2H), 7.15 (d, 1H), 7.04 (d, 2H), 5.13 (d, 1H), 3.15 (s, 1H), 3.08 (dd, 1H), 2.95-3.05 (m, 2H), 2.94 (t, 1H), 2.89 (t, 1H), 2.79 (m, 1H), 2.53 (m, 1H), 2.42 (dd, 1H), 1.96 (m, 1H), 1.27 (s, 9H), 1.03 (m, 1H), 0.88 (m, 1H), 0.84 (m, 1H), 0.71 (m, 1H).

### k) (3S,4S,5R)-3-(4-Amino-benzyl)-5-[1-(3-tert-butyl-phenyl)-cyclopropylamino]-1,1-dioxo-hexahydro-1lambda*6*-thiopyran-4-ol hydrochloride

A solution of (3S*,4S*,5R*)-3-(4-azido-benzyl)-5-[-(3-tert-butyl-phenyl)-cyclopropylamino]-1,1-dioxo-hexahydro-1lambda*6*-thiopyran-4-ol in MeOH (20 mL) is hydrogenated (1 atm H₂) at 25° C. over 10% Pd—C (0.08 g) for 1 h. The catalyst is filtered off over Celite and the filtrate is evaporated to yield the racemic title compound as a light yellow oil. The racemate is separated by preparative chromatography on a Chiralpak AD-H column with hexane-EtOH 1:1 to yield the optically pure (3R,4R,5S)- and (3S,4S,5R)-diastereoisomer (peak 2) as a colorless foam: TLC (hexane/EtOAc 1:2) Rf=0.20; ESIMS [M+H]⁺=443; ¹H-NMR (400 MHz, CDCl₃): δ7.41 (s, 1H), 7.31 (m, 2H), 7.20 (d, 1H), 6.92 (d, 2H), 6.61 (d, 2H), 3.63 (s, 2H), 3.22 (m, 1H), 3.03 (dd, 1H), 2.81 (m, 2H), 2.55 (m, 3H), 2.26 (m, 2H), 1.64 (s, 2H), 1.34 (s, 9H), 1.10 (m, 2H), 0.89 (m, 2H).

### General HPLC Information Concerning Examples 23 to 34h

HPLC method A (Rt_{A}):

HPLC-column dimensions: 50×4.6 mm

HPLC-column type: Chromolith Speed ROD RP-18e, 2 μm

HPLC-eluent: A) water+0.1 Vol.-% TFA
B) ACN+0.1 Vol.-% TFA

HPLC-gradient: 10-100% B in 3 min+1 min 100%B, flow=4 ml/min

HPLC method B (Rt_{B}):

HPLC-column dimensions: 125×4 mm

HPLC-column type: MN Nucleodur C18 Pyramid, 5 μm

HPLC-eluent: A) water+0.1 Vol.-% TFA
B) ACN+0.1 Vol.-% TFA

HPLC-gradient: 5% B to 100% B in 20 min flow=1 ml/min

HPLC method B2 (Rt_{B2}):

HPLC-column dimensions: 125×4 mm

HPLC-column type: MN Nucleodur C18 Pyramid, 5 μm

HPLC-eluent: A) water
B) ACN

HPLC-gradient: 5% B to 100% B in 20 min flow=1 ml/min

HPLC method C (Rt_{C}):

HPLC-column dimensions: 2.1×50 mm

HPLC-column type: SunFire C₁₈, 5 μm

HPLC-eluent: A) ACN
B) water+0.1 Vol.-% TFA

HPLC-gradient: 20-95% A in 3.5 min+95% A in 0.5 min+95-20% A in 0.5 min flow=0.8 ml/min

HPLC method D (Rt_{D}):

HPLC-column dimensions: 5×100 mm

HPLC-column type: Machery-Nagel LiChrospher RP-18, 5 μm

HPLC-eluent: A) ACN
B) water+0.1 Vol.-% TFA

HPLC-gradient: 10-100% A in 5 min, flow=1.5 ml/min

HPLC method E (Rt_{E}):

HPLC-column dimensions: 4.6×100 mm

HPLC-column type: XTerra MS C₁₈, 3.5 μm

HPLC-eluent: A) water+0.1 Vol.-% TFA
B) ACN+0.1 Vol.-% TFA

HPLC-gradient: 5% B for 1 min, 5-50% B in 4 min, 50-100% B in 2 min, flow=0.9 ml/min

HPLC method F (Rt_{F}):

HPLC-column dimensions: 150×4.6 mm

HPLC-column type: Luna (Phenomenex) C18, 5 μm

HPLC-eluent: A) water+0.1 Vol.-% TFA
B) ACN+0.1 Vol.-% TFA

HPLC-gradient: 90% A) 5 min, 90% B) 3 min. 90% A) 2 min. flow=6 ml/min

HPLC method G (Rt_{BG}):

HPLC-column dimensions: 50×4.6 mm

HPLC-column type: Chromolith SpeedROD RP-18e, Chromolith

HPLC-eluent: A) water+0.1 Vol.-% TFA
B) ACN+0.1 Vol.-% TFA

HPLC-gradient: 95:5 A):B) 1 min; 5:95 A):B) 8 min; 95:5 A):B) 2 min. flow=2 ml/min

### Example 23

### 3S*,4S*,5R*)-3-[4-Amino-3-fluoro-5-(3,3,3-trifluoro-propyl)-benzyl]-5-(3-tert-butyl-benzylamino)-1,1-dioxo-hexahydro-1lambda*6*-thiopyran-4-ol dihydro-chloride

### a) (3aR*,7S*,7aS*)-3-(3-tert-Butyl-benzyl)-7-(3-fluoro-4-nitro-benzyl)-5,5-dioxo-hexa-hydro-1-oxa-5lambda*6*-thia-3-aza-inden-2-one

To a suspension of (3R*,4S*,5S*)-3-(3-tert-butyl-benzylamino)-5-(3-fluoro-4-nitro-benzyl)-1,1-dioxo-hexahydro-1lambda*6*-thiopyran-4-ol hydrochloride (example 1h)) (4.4 g, 8.7 mmol) in ACN is added at 25° C. the DIPEA and the carbonyl-diimidazole. After addition of a catalytic amount of DMAP, the clear reaction mixture is kept at 25° C. for 16 h. The reaction mixture is poured onto ice-water, acidified with 4N HCl and after stirring for 10 min filtered. The precipitate is collected and washed with H₂O and Et₂O, dried under reduced pressure for 6 h at 50° C. to yield the title product as light yellow crystals: TLC (toluene-THF 1:1) Rf=0.66; HPLC Rt_{A}=2.25 min; ESIMS [M+NH₃+H]⁺=508.

### b) (3aR,7S,7aS)-7-(4-Amino-3-fluoro-benzyl)-3-(3-tert-butyl-benzyl)-5,5-dioxo-hexa-hydro-1oxa-5lambda*6*-thia-3-aza-inden-2-one

A solution of (3aR*, 7S*,7aS*)-3-(3-tert-butyl-benzyl)-7-(3-fluoro-4-nitro-benzyl)-5,5-dioxo-hexahydro-1-oxa-5lambda*6*-thia-3-aza-inden-2-one (4.0 g, 8.1 mmol) in THF (150 mL) is hydrogenated over 10% Pd/C (300 mg) at 45° C. and 1 bar. After 16 h the catalyst is filtered off over Celite and the filtrate is evaporated. The residue is recrystallized from THF-hexane to yield the title compound as beige crystals: TLC (toluene-THF 1:1) Rf=0.51; HPLC Rt_{A}=1.94 min; ESIMS [M+H+NH₃]⁺=478.

### c) (3aR*,7S*,7aS*)-7-(4-Amino-3-fluoro-5-iodo-benzyl)-3-(3-tert-butyl-benzyl)-5,5-dioxo-hexahydro-1-oxa-5lambda*6*-thia-3-aza-inden-2-one

To a suspension of (3aR,7S,7aS)-7-(4-amino-3-fluoro-benzyl)-3-(3-tert-butyl-benzyl)-5,5-di-oxo-hexahydro-1oxa-5lambda*6*-thia-3-aza-inden-2-one (3.0 g, 6.5 mmol) in CH₂Cl₂-MeOH 3:1 (180 mL) is added CaCO₃ (1.96 g, 19.4 mmol) and the benzyltrimethylammonium dichloroiodide (3.47, 947 mmol) at 25° C. under argon. The reaction mixture is heated at reflux for 16 h, diluted with CH₂Cl₂ and washed with ice-water, cold sodium thiosulfate solution and brine, dried over MgSO₄, filtered and evaporated. The residue is purified by flash-chromatography on silica gel (hexane-CH₂Cl₂-EtOAc 5:2:2 to 0:2:2) and crystallized from EtOAc-tBuOMe-hexane to yield the title compound as light yellow crystals: TLC (hexane-EtOAc 1:1) Rf=0.39; HPLC Rt_{A}=2.31 min; ESIMS [M+H+NH₃]⁺=604.

### d) (3aR*,7S*,7aS*)-7-(4-Amino-3-fluoro-5-trifluoroprop-1-ynyl-benzyl)-3-(3-tert-butyl-benzyl)-5,5-dioxo-hexahydro-1-oxa-5lambda*6*-thia-3-aza-inden-2-one

To a solution of 3,3,3-trifluoropropyne (0.495 g, 5.0 mmol) in anhydrous THF (10 mL) is added under argon at −78° C. a 1.6 M solution of nBuLi in hexane (3.2 mL, 5.0 mmol). After stirring for 0.5 h at −78° C. a 1 M solution of ZnCl₂ in Et₂O (15 mL, 15 mmol) is added and the reaction mixture is warmed up to 25° C. over a period of 2 h. To the reaction mixture is added (3aR*,7S*,7aS*)-7-(4-amino-3-fluoro-5-iodo-benzyl)-3-(3-tert-butyl-benzyl)-5,5-dioxo-hexa-hydro-1-oxa-5lambda*6*-thia-3-aza-inden-2-one (0.77 g, 1.25 mmol) and Pd(PPh₃)₄ (0.073 g, 0.06 mmol). After stirring for 18 h at 60° C., the reaction mixture is poured into ice-water and extracted with EtOAc. The combined organic layers are washed with 10% aqueous K₂CO₃ solution and brine, dried over MgSO₄, filtered and evaporated. The residue is purified by preparative HPLC (Sun Five C₁₈ OBD 5 μm, 100×30, 5-100% ACN in water+0.1% TFA gradient, 25 min) to yield the title compound as beige solid after basification with K₂CO₃ solution and re-extraction with EtOAc: TLC (hexane-EtOAc 1:1) Rf=0.51; HPLC Rt_{A}=2.46 min; ESIMS [M+H+NH₃]⁺=570.

### e) (3aR*,7S*,7aS*)-7-[4-Amino-3-fluoro-5-(3,3,3-trifluoro-propyl)-benzyl]-3-(3-tert-butyl-benzyl)-5,5-dioxo-hexahydro-1-oxa-5lambda*6*-thia-3-aza-inden-2-one

A solution of (3aR*,7S*,7aS*)-7-(4-amino-3-fluoro-5-trifluoroprop-1-ynyl-benzyl)-3-(3-tert-butyl-benzyl)-5,5-dioxo-hexahydro-1-oxa-5lambda*6*-thia-3-aza-inden-2-one (0.12 g, 0.21 mmol) in MeOH-THF 2:1 (9 mL) is hydrogenated over 10% Pd/C (40 mg) at 25° C. and 1 bar hydrogen for 2 h. The catalyst is filtered off over Celite and the filtrate is evaporated. The residue is purified by preparative HPLC (Sun Five C₁₈ OBD 5 μm, 100×30, 5-100% ACN in water+0.1% TFA gradient, 25 min) to yield the title compound as a beige foam after basification with K₂CO₃ solution and re-extraction with EtOAc: TLC (hexane-EtOAc 1:1) Rf=0.54; HPLC Rt_{A}=2.38 min; ESIMS [M+H+NH₃]⁺=574.

### f) (3S*,4S*,5R*)-3-[4-Amino-3-fluoro-5-(3,3,3-trifluoro-propyl)-benzyl]-5-(3-tert-butyl-benzylamino)-1,1-dioxo-hexahydro-1lambda*6*-thiopyran-4-ol dihydrochloride

To a solution of (3aR*,7S*,7aS*)-7-[4-amino-3-fluoro-5-(3,3,3-trifluoro-propyl)-benzyl]3-(3-tert-butyl-benzyl)-5,5-dioxo-hexahydro-1-oxa-5lambda*6*-thia-3-aza-inden-2-one (0.068 g, 0.12 mmol) in anhydrous THF (4 mL) is added KOSi(CH₃)₃ (0.068 g, 0.48 mmol) and the reaction mixture is heated for 2 h at 60° C. The reaction mixture is added to cold 10% aqueous K₂CO₃ solution and extracted with EtOAc. The combined organic layers are washed with 10% aqueous K₂CO₃ solution and brine, dried over MgSO₄, filtered and evaporated. The residue is converted into the dihydrochloride salt with 1 N HCl in Et₂O and the precipitated salt is dried under reduced pressure to yield the title compound as a light yellow solid: TLC (CH₂Cl₂-MeOH 19:1) Rf=0.50; HPLC Rt_{A}=1.86 min; ESIMS [M+H]⁺=531.

### Example 24

### 3S*,4S*,5R*)-3-(4-Amino-3-cyclopropyl methyl-5-fluoro-benzyl)-5-(3-tert-butyl-benzylamino)-1,1-dioxo-hexahydro-1lambda*6*-thiopyran-4-ol trifluoroacetate

### a) (3aR*,7S*,7aS*)-7-(3-Allyl-4-amino-5-fluoro-benzyl)-3-(3-tert-butyl-benzyl)-5,5-dioxo-hexahydro-1-oxa-5lambda*6*-thia-3-aza-inden-2-one

To a degassed solution of (3aR*,7S*,7aS*)-7-(4-amino-3-fluoro-5-iodo-benzyl)-3-(3-tert-butyl-benzyl)-5,5-dioxo-hexahydro-1-oxa-5lambda*6*-thia-3-aza-inden-2-one (example 23c)) (0.300 g, 0.435 mmol) in DME-water 10:1 (22 mL) is added tri-potassium phosphate monohydrate (0.185 g, 0.870 mmol), allylboronic acid pinacol ester (0.205 ml, 1.09 mmol), Pd₂(dba)₃ (0.023 g, 0.022 mmol) and Ph₅FcP(tBu)₂ (CTC-Q-PHOS) (0.033 g, 0.043 mmol). The reaction mixture is stirred overnight at 80° C. After addition of saturated Na₂CO₃ solution, the reaction mixture is extracted with EtOAc, and the extract is washed with brine, dried over Na₂SO₄, filtered and concentrated. The crude product is purified by flash-chromatography on silica gel (hexane-EtOAc 1:0 to 7:3) to yield the title compound as an orange solid: TLC (hexane-EtOAc 1:1) Rf=0.51; HPLC Rt_{D}=5.55 min; ESIMS [M+H]⁺=501.

### b) (3S*,4S*,5R*)-3-(3-Allyl-4-amino-5-fluoro-benzyl)-5-(3-tert-butyl-benzylamino)-1,1-dioxo-hexahydro-1lambda*6*-thiopyran-4-ol

To a solution of (3aR*,7S*,7aS*)-7-(3-allyl-4-amino-5-fluoro-benzyl)-3-(3-tert-butyl-benzyl)-5,5-dioxo-hexahydro-1-oxa-5lambda*6*-thia-3-aza-inden-2-one (0.151 g, 0.302 mmol) in THF (3 mL) is added potassium trimethylsilanolate (0.086 g, 0.603 mmol). The reaction mixture is stirred for 2 h at 80° C., then concentrated and purified by flash-chromatography on silica gel (hexane-EtOAc 1:0 to 1:1) to yield the title compound as a pink foam: TLC (hexane-EtOAc 1:1) Rf=0.20; HPLC Rt_{D}=3.97 min; ESIMS [M+H]⁺=475.

### c) (3S*,4S*,5R*)-3-(4-Amino-3-cyclopropyl methyl-5-fluoro-benzyl)-5-(3-tert-butyl-benzylamino)-1,1-dioxo-hexahydro-1lambda*6*-thiopyran-4-ol trifluoroacetate

To a solution of (3S*,4S*,5R*)-3-(3-Allyl-4-amino-5-fluoro-benzyl)-5-(3-tert-butyl-benzyl-amino)-1,1-dioxo-hexahydro-1lambda*6*-thiopyran-4-ol (0.1 g, 0.211 mmol) and palladium(II)acetylacetonate (0.128 mg, 0.421 mmol) in Et₂O (5 mL) is added at 0° C. a solution of diazomethane in ether prepared at 0° C. with 40% aq. KOH, Et₂O and N-nitroso-N-methylurea (0.130 g, 1.26 mmol). After stirring for 2 days at 0° C. and a second addition of a solution of diazomethane, the reaction mixture is filtered, and the filtrate is evaporated. The title compound is purified by preparative HPLC (Sun Five C18 OBD 5 μm, 100×30, 5-100% ACN in water+0.1% TFA gradient, 25 min) and obtained as a colourless solid: TLC (hexane-EtOAc 1:1) Rf=0.27; HPLC Rt_{D}=4.06 min; ESIMS [M+H]⁺=489.

### Example 25

### (3S*,4S*,5R*)-3-[4-Amino-3-fluoro-5-(3-methyl-butyl)-benzyl]-5-(3-tert-butyl-benzylamino)-1,1-dioxo-hexahydro-1lambda*6*-thiopyran-4-ol trifluoroacetate

### a) (3aR*,7S*,7aS*)-7-[4-Amino-3-fluoro-5-(3-methyl-but-1-ynyl)-benzyl]-3-(3-tert-butyl-benzyl)-5,5-dioxo-hexahydro-1-oxa-5lambda*6*-thia-3-aza-inden-2-one

To a solution of (3aR*,7S*,7aS*)-7-(4-amino-3-fluoro-5-iodo-benzyl)-3-(3-tert-butyl-benzyl)-5,5-dioxo-hexahydro-1-oxa-5lambda*6*-thia-3-aza-inden-2-one (example 23c)) (0.2 g, 0.341 mmol) in anhydrous THF (5 mL) are added NEt₃ (5 mL) and Pd(PPh₃)₂Cl₂ (0.024 g, 0.034 mmol). After stirring for 5 min, CuI (0.013 g, 0.068 mmol) and 3-methylbutyne (0.046 g, 0.682 mmol) are added. The dark mixture is stirred for 1 h at 25° C., filtered and concentrated. The crude product is purified by flash-chromatography on silica gel (cyclohexane-EtOAc 1:0 to 1:1) to yield the title compound: HPLC Rt_{c}=6.13 min; ESIMS [M−H]⁻=525.

### b) (3S*,4S*,5R*)-3-[4-Amino-3-fluoro-5-(3-methyl-but-1-ynyl)-benzyl]-5-(3-tert-butyl-benzylamino)-1,1-dioxo-hexahydro-1lambda*6*-thiopyran-4-ol

To a solution of (3aR*,7S*,7aS*)-7-[4-amino-3-fluoro-5-(3-methyl-but-1-ynyl)-benzyl]-3-(3-tert-butyl-benzyl)-5,5-dioxo-hexahydro-1-oxa-5lambda*6*-thia-3-aza-inden-2-one (0.115 g, 0.218 mmol) in anhydrous THF (10 mL) is added KOSi(CH₃)₃ (0.157 g, 1.09 mmol), and the reaction mixture is heated for 0.5 h at 80° C. The reaction mixture is acidified with 1 N aq. HCl in Et₂O until pH˜4 and evaporated under reduced pressure. The residue is extracted with EtOAc. The combined organic layers are washed with saturated aq. NaHCO₃ solution and brine, dried over MgSO₄, filtered and evaporated. The residue is dried under reduced pressure to yield the title compound as a light yellow solid: HPLC Rt_{c}=4.86 min; ESIMS [M+H]⁺=501, [M−H]⁻=499.

### c) (3S*,4S*,5R*)-3-[4-Amino-3-fluoro-5-(3-methyl-butyl)-benzyl]-5-(3-tert-butyl-benzylamino)-1,1-dioxo-hexahydro-1lambda*6*-thiopyran-4-ol trifluoroacetate

To a solution of (3S*,4S*,5R*)-3-[4-amino-3-fluoro-5-(3-methyl-but-1-ynyl)-benzyl]-5-(3-tert-butyl-benzylamino)-1,1-dioxo-hexahydro-1lambda*6*-thiopyran-4-ol (0.05 g, 0.1 mmol) in MeOH (5 mL) are added NiCl₂-6H₂O (0.024 g, 0.1 mmol) and at 0-5° C. NaBH₄ (0.015 g, 0.399 mmol) in small portions over a period of 15 min. After stirring for 20 min at 0-5° C. the reaction is quenched by addition of H₂O (0.5 mL). The reaction mixture is filtered through a plug of Celite and evaporated. The residue is taken up in EtOAc and washed with a saturated solution of Na₂CO₃ and brine, dried over MgSO₄ and evaporated. The title compound is obtained after purification by preparative HPLC as a colourless foam: HPLC Rt_{c}=4.53 min; ESIMS [M+H]⁺=505, [M−H]⁻=503.

### Example 26

### (3S*,4S*,5R*)-3-(4-Amino-3-cyclopropyl-5-fluoro-benzyl)-5-(3-tert-butyl-benzylamino)-1,1-dioxo-hexahydro-1lambda*6*-thiopyran-4-ol trifluoroacetate

### a) (3aR*,7S*,7aS*)-7-(4-Amino-3-cyclopropyl-5-fluoro-benzyl)-3-(3-tert-butyl-benzyl)-5,5-dioxo-hexahydro-1-oxa-5lambda*6*-thia-3-aza-inden-2-one

To a solution of (3aR*,7S*,7aS*)-7-(4-amino-3-fluoro-5-iodo-benzyl)-3-(3-tert-butyl-benzyl)-5,5-dioxo-hexahydro-1-oxa-5lambda*6*-thia-3-aza-inden-2-one (example 23c)) (0.08 g, 0.136 mmol) in toluene-water20:1 (1.6 mL) are added cyclopropyl boronic acid (0.019 g, 0.205 mmol), tri-potassium phosphate monohydrate (0.110 g, 0.477 mmol) and dichloro-bis-(triphenylphosphine)palladium (0.005 g,0.007 mmol). After stirring for 3 h at 105° C., the reaction mixture is quenched with water, extracted with EtOAc, washed with brine, dried over Na₂SO₄, filtered and concentrated. The crude product is purified by flash-chromatography on silica gel (hexane-EtOAc 1:0 to 1:1) to yield the title compound: HPLC Rt_{D}=5.42 min; ESIMS [M+NH₄]⁺=518.

### b) (3S*,4S*,5R*)-3-(4-Amino-3-cyclopropyl-5-fluoro-benzyl)-5-(3-tert-butyl-benzylamino)-1,1-dioxo-hexahydro-1lambda*6*-thiopyran-4-ol trifluoroacetate

To a solution of (3aR*,7S*,7aS*)-7-(4-Amino-3-cyclopropyl-5-fluoro-benzyl)-3-(3-tert-butyl-benzyl)-5,5-dioxo-hexahydro-1-oxa-5lambda*6*-thia-3-aza-inden-2-one in THF (1 mL) is added potassium trimethyl silanolate (0.035 g, 0.244 mmol), and the reaction mixture is stirred for 2 h at 80° C. and then evaporated. The residue is quenched with saturated aq. NaHCO₃ solution. The mixture is extracted with EtOAc, the combined layers are washed with brine, dried over Na₂SO₄, filtered and concentrated. The crude product is purified by preparative HPLC (Sun Five C18 OBD 5 μm, 100×30, 5-100% ACN in water+0.1% TFA gradient, 25 min) to yield to a colourless solid: HPLC Rt_{D}=3.76 min; ESIMS [M+H]⁺=475.

### Examples 26a -26d

The compounds listed in Table 4 can be prepared by a procedure analogous to that used in example 26.

TABLE 4

| Example | Compound of the formula 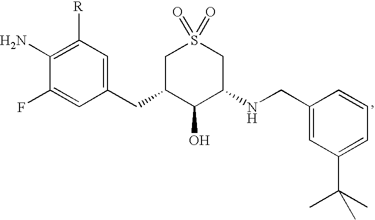 in which R | HPLCRt [min]Method | ESIMS |
|---|---|---|---|
| 26a | 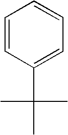 | 4.362C | [M + H]⁺ = 511 |
| 26b | 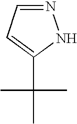 | 8.68B | [M + H]⁺ = 501 |
| 26c | 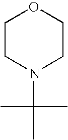 | 1.66A | [M + H]⁺ = 520 |
| 26d | 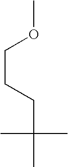 | 3.707C | [M + H]⁺ = 507 |

### Example 27

### (3S*,4S*,5R*)-3-(4-Amino-3-fluoro-5-methoxymethyl-benzyl)-5-(3-tert-butyl-benzylamino)-1,1-dioxo-hexahydro-1lambda*6*-thiopyran-4-ol trifluoroacetate

### a) 4-Amino-3-fluoro-5-vinyl-benzoic acid methyl ester

To a solution of 4-amino-3-bromo-5-fluoro-benzoic acid methyl ester (example 15a)) (15 g, 60.5 mmol), Cs₂CO₃ (59.1 g, 181 mmol) and potassium vinyl trifluoroborate (12.8 g, 90.7 mmol) in THF-H₂O 9:1 (605 mL) is added under argon Pd(PPh₃)₂Cl₂ (0.848 g, 1.21 mmol), and the reaction mixture is heated at reflux for 1 day. The reaction mixture is diluted with water (30 mL) and extracted with EtOAc. The combined organic layers are washed with water and brine, dried over MgSO₄, filtered and evaporated. The title compound is obtained after flash-chromatography on silica gel (cyclohexane to cyclohexane-EtOAc 4:1) as a white solid: HPLC Rt_{c}=3.98 min; ESIMS [M+H]⁺=196, [M−H]⁻=194. ¹H-NMR (400 MHz, CDCl₃): δ 7.8 (s, 1H), 7.6 (d, 1H), 6.7 (q, 1H), 5.75 (d, 1H), 5.4 (d, 1H), 4.1 (s, 2H), 3.85 (s, 3H).

### b) 4-Amino-3-fluoro-5-hydroxymethyl-benzoic acid methyl ester

A solution of 4-amino-3-fluoro-5-vinyl-benzoic acid methyl ester (1.5 g, 10.2 mmol) is ozonized in EtOAc (30 mL) at −78° C. for 30 min and then NaBH₄ (0.775 g, 20.5 mmol) is added. The reaction mixture is stirred for 1 h at 25° C. The reaction mixture is quenched with ice/1N aq. HCl, extracted with EtOAc, washed with water and brine, dried over Na₂SO₄, filtered and concentrated. The title compound is obtained after flash-chromatography on silica gel (cyclohexane-EtOAc 1:0 to 1:1) as an orange foam: HPLC Rt_{c}32 2.28 min; ESIMS [M+H]⁺=200, [M−H]⁻=198.

### c) 4-Amino-3-bromomethyl-5-fluoro-benzoic acid methyl ester

A solution of 4-amino-3-fluoro-5-hydroxymethyl-benzoic acid methyl ester (0.450 g, 2.26 mmol), CBr₄ (1.12 g, 3.39 mmol) and PPh₃ (0.889 g, 3.39 mmol) are suspended in THF (20 mL) at 25° C. and stirred at 25° C. for 1 h. The reaction mixture is directly used as such for the next step.

### d) 4-Amino-3-fluoro-5-methoxymethyl-benzoic acid methyl ester

To the previous mixture, MeOH is added until disappearance of the precipitate and the reaction mixture is refluxed for 30 min. After removal of the solvents the title compound is obtained after flash-chromatography on silica gel (cyclohexane-EtOAc 1:0 to 4:1) as a yellow solid: HPLC Rt_{c}=4.89 min; ESIMS [M+H]⁺=214, [M−H]⁻=212.

### e) 3-Fluoro-5-methoxymethyl-4-nitro-benzoic acid methyl ester

To a solution of 50% H₂O₂ (0.67 mL, 10.9 mmol) in CH₂Cl₂ at 0° C. is added dropwise trifluoroacetic acid anhydride (1.77 mL, 12.8 mmol). The solution is warmed to 25° C. and 4-amino-3-fluoro-5-methoxymethyl-benzoic acid methyl ester (0.35 g, 1.64 mmol) dissolved in CH₂Cl₂ (5 mL) is added dropwise. The reaction mixture is refluxed for 1 h at 45° C. and quenched at 0° C. with aq. saturated Na₂SO₃. The organic layer is extracted with EtOAc, washed with water and brine, dried over Na₂SO₄, filtered and concentrated. The title compound is obtained after flash-chromatography on silica gel (hexane-EtOAc 1:0 to 1:1) to yield the desired compound as a yellow solid: HPLC Rt_{c}=4.59 min; ESIMS [M+H]⁺=260.

### f) (3-Fluoro-5-methoxymethyl-4-nitro-phenyl)-methanol

The title compound is prepared in analogous manner as described for example 15f) from 3-fluoro-5-methoxymethyl-4-nitro-benzoic acid methyl ester (example 30e): HPLC Rt_{c}=3.13 min; ESIMS [M+NH₄]⁺=233.

### g) 5-Bromomethyl-1-fluoro-3-methoxymethyl-2-nitro-benzene

To a solution of PBr₃ (0.328 mL, 3.49 mmol) in Et₂O (5 mL) is added dropwise at 0° C. (3-fluoro-5-methoxymethyl-4-nitro-phenyl)-methanol (0.30 g, 1.39 mmol) dissolved in Et₂O (2 mL). The reaction is stirred overnight at 25° C., cooled to 0° C. and quenched with MeOH. After 10 min stirring, the reaction mixture is diluted with saturated aq. NaHCO₃ (100 mL) and extracted with EtOAc, washed with brine, dried over Na₂SO₄, filtered and concentrated. The title compound is obtained after flash-chromatography on silica gel (hexane-EtOAc 1:0 to 4:1) as a yellow syrup. HPLC Rt_{c}=4.91 min.

### h) 5-tert-Butoxycarbonylamino-3-(3-fluoro-5-methoxymethyl-4-nitro-benzyl)-4-oxo-tetrahydro-thiopyran-3-carboxylic acid allyl ester

The title compound is prepared in analogous manner as described for example 1c) from 5-bromomethyl-1-fluoro-3-methoxymethyl-2-nitro-benzene and 5-tert-butoxycarbonylamino-4-oxo-tetrahydro-thiopyran-3-carboxylic acid allyl ester (example 1b): HPLC Rt_{c}=5.91 min; ESIMS [M+H]⁺=529.

### i) [(3R,5S)-5-(3-Fluoro-5-methoxymethyl-4-nitro-benzyl)-4-oxo-tetrahydro-thiopyran-3-yl]-carbamic acid tert-butyl ester

The title compound is prepared in analogous manner as described for example 1d) from 5-tert-butoxycarbonylamino-3-(3-fluoro-5-methoxymethyl-4-nitro-benzyl)-4-oxo-tetrahydro-thio-pyran-3-carboxylic acid allyl ester: HPLC Rt_{c}=5.65 min; ESIMS [M+NH₄]⁺=445, [M−H]⁺=426.

### j) [(3R*,4R*,5S*)-5-(3-Fluoro-5-methoxymethyl-4-nitro-benzyl)-4-hydroxy-tetrahydro-thiopyran-3-yl]-carbamic acid tert-butyl ester and [(3R*,4S*,5S*)-5-(3-Fluoro-5-methoxymethyl-4-nitro-benzyl)-4-hydroxy-tetrahydro-thiopyran-3-yl]-carbamic acid tert-butyl ester

The title compound is prepared in analogous manner as described for example 1e) from [(3R,5S)-5-(3-fluoro-5-methoxymethyl-4-nitro-benzyl)-4-oxo-tetrahydro-thiopyran-3-yl]-carbamic acid tert-butyl ester: HPLC Rt_{c}=5.01 min; ESIMS [M+H-Boc]⁺=330.

### k) [(3R*,4S*,5S*)-5-(3-Fluoro-5-methoxymethyl-4-nitro-benzyl)-4-hydroxy-1,1-dioxo-hexahydro-1lambda*6*-thiopyran-3-yl]-carbamic acid tert-butyl ester

The title compound is prepared in analogous manner as described for example 1f) from [(3R,5S)-5-(3-fluoro-5-methoxymethyl-4-nitro-benzyl)-4-oxo-tetrahydro-thiopyran-3-yl]-carbamic acid tert-butyl ester: HPLC Rt_{c}=4.20 min; ESIMS [M+NH₄]⁺=480.

### l) (3R*,4S*,5S*)-3-Amino-5-(3-fluoro-5-methoxymethyl-4-nitro-benzyl)-1,1-dioxo-hexahydro-1lambda*6*-thiopyran-4-ol

To a solution of [(3R*,4S*,5S*)-5-(3-fluoro-5-methoxymethyl-4-nitro-benzyl)-4-hydroxy-1,1-dioxo-hexahydro-1lambda*6*-thiopyran-3-yl]-carbamic acid tert-butyl ester (0.20 g, 0.432 mmol) in CH₂Cl₂ (20 mL) is added 4N HCl in dioxane (3.24 mL, 13.0 mmol). The solution is stirred for 1 h at 25° C. and evaporated. After basification with 1N aq. NaOH the title compound is extracted with EtOAc. The combined organic layers are washed with water and brine, dried over Na₂SO₄, filtered and concentrated: HPLC Rt_{c}=2.57 min; ESIMS [M+H]⁺=363.

### m) (3R*,4S*,5S*)-3-(3-tert-Butyl-benzylamino)-5-(3-fluoro-5-methoxymethyl-4-nitro-benzyl)-1,1-dioxo-hexahydro-1lambda*6*-thiopyran-4-ol

To a solution of (3R*,4S*,5S*)-3-amino-5-(3-fluoro-5-methoxymethyl-4-nitro-benzyl)-1,1-dioxo-hexahydro-1lambda*6*-thiopyran-4-ol (0.14 g, 0.386 mmol) in MeOH—CH₂Cl₂ 1:1 (20 mL) is added NaOAc (0.095 g, 1.16 mmol). After 5 min, 3-tert-butyl-benzaldehyde (0.188 g, 1.16 mmol) is added and the reaction mixture is stirred at 25° C. for 16 h. NaBH₃CN (0.049 g, 0.773 mmol) is added and stirring for is continued for 1 h. The reaction mixture is filtered and concentrated. The title compound is obtained after purification by flash-chromatography on silica gel (cyclohexane-EtOAc 1:0 to 1:1) as a white foam: HPLC Rt_{c}=4.29 min; ESIMS [M+H]⁺=509.

### n) (3S*,4S*,5R*)-3-(4-Amino-3-fluoro-5-methoxymethyl-benzyl)-5-(3-tert-butyl-benzylamino)-1,1-dioxo-hexahydro-1lambda*6*-thiopyran-4-ol trifluoroacetate

To a solution of (3R*,4S*,5S*)-3-(3-tert-butyl-benzylamino)-5-(3-fluoro-5-methoxymethyl-4-nitro-benzyl)-1,1-dioxo-hexahydro-1lambda*6*-thiopyran-4-ol (0.16 g, 0.315 mmol) in MeOH (10 mL) is added NiCl₂-6H₂O (0.075 g, 0.315 mmol) and at 0-5° C. NaBH₄ (0.048 g, 1.26 mmol) in small portions over a period of 15 min. After stirring for 20 min at 0-5° C. the reaction is quenched by addition of H₂O (0.5 mL). The reaction mixture is filtered through a plug of Celite and evaporated. The residues is taken up in EtOAc and washed NaHCO₃ solution and brine, dried over MgSO₄ and evaporated. The title compound is obtained after purification by prep. HPLC as a white foam: HPLC Rt_{c}=3.65 min; ESIMS [M+H]⁺=479, [M−H]⁻=477.

### Examples 27a -27b

The compounds listed in Table 5 can be prepared by a procedure analogous to that used in example 27.

TABLE 5

| Example | Compound of the formula 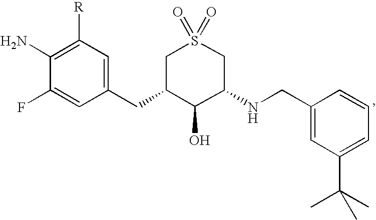 in which R | HPLCRt [min]Method | ESIMS |
|---|---|---|---|
| 27a | 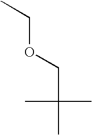 | 3.93C | [M + H]⁺ = 492 |
| 27b | 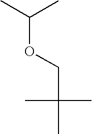 | 4.15C | [M + H]⁺ = 507 |

### Example 28

### (3S*,4S*,5R*)-3-(4-Amino-3-ethoxy-5-fluoro-benzyl)-5-(3-tert-butyl-benzyl-amino)-1,1-dioxo-hexahydro-1lambda*6*-thiopyran-4-ol dihydrochloride

### a) 3,5-Difluoro-4-nitro-benzoic acid methyl ester

Thionylchloride (1.0 mol, 134 mL) is added to MeOH (400 mL) at −10° C. to −20° C. over a period of 0.5 h. To this solution is added 3,5-difluoro-4-nitro-benzonitrile (33.5 g, 180 mmol) and the reaction mixture is allowed to warm to 25° C. and stirred overnight at 25° C. Afterwards the temperature is slowly increased to 50° C. over 2 h and the evolving gas is trapped in a gas washer. Finally the reaction mixture is heated at reflux for 2 h. and finally to reflux. The cooled reaction mixture was filtered and concentrated. The crude product is dissolved in EtOAc and washed with cold NaHCO₃ solution and brine, dried over MsSO₄, filtered and concentrated. The residue is crystallized from EtOAc-hexane to yield the title compound as an orange solid: TLC (hexane-EtOAc 3:1) Rf=0.38; HPLC Rt_{A}=1.74 min; ¹H-NMR (400 MHz, CDCl₃): δ 7.76 (d, 2H), 3.98 (s, 3H).

### b) (3,5-Difluoro-4-nitro-phenyl)-methanol

To a solution of 3,5-difluoro-4-nitro-benzoic acid methyl ester (10.95 g, 50 mmol) in THF (250 mL) is added at 0-5° C. under argon a 1M DIBAL solution in hexane (165 mL, 165 mmol) within 1.5 h. The reaction mixture is stirred for 2.5 h at 0-5° C. before it is added to 200 mL cold 1M aqueous potassium tartrate solution under ice-cooling. After stirring the reaction mixture for 0.5 h at 25° C., the aqueous phase is extracted with EtOAc. Combined organic extracts are washed with brine, dried over MgSO₄, filtered and evaporated to provide the title compound as a yellow solid: TLC (hexane-EtOAc 1.1) Rf=0.35; HPLC Rt_{A}=1.31 min; ¹H-NMR (400 MHz, CDCl₃): δ 7.12 (d, 2H), 4.77 (s, 2H).

### c) 5-Bromomethyl-1,3-difluoro-2-nitro-benzene

To a solution of PBr₃ (7.04 mL, 73 mmol) in Et₂O (200 mL) is added under Argon at 0° C. a solution of (3,5-difluoro-4-nitro-phenyl)-methanol (9.3 g, 48.7 mmol) in Et₂O (200 mL). The reaction mixture is allowed to warm to 25° C. and stirred for 24 h at 25° C. After the addition of MeOH (5 mL) at 0° C., the reaction mixture is poured onto cold NaHCO₃ solution and the product is extracted with EtOAc. Combined organic extracts are washed with NaHCO₃-solution and brine, dried over MgSO₄, filtered and concentrated to provide the title compound after filtration through a plug of silicagel with hexane-EtOAc 3:1 as a yellow solid: TLC (hexane-EtOAc 3:1) Rf=0.40; HPLC Rt_{A}=2.01 min; ¹H-NMR (400 MHz, CDCl₃): δ 7.14 (d, 2H), 4.40 (s, 2H).

### d) 5-tert-Butoxycarbonylamino-3-(3,5-difluoro-4-nitro-benzyl)-4-oxo-tetrahydro-thio-pyran-3-carboxylic acid allyl ester

To a solution of 5-bromomethyl-1,3-difluoro-2-nitro-benzene (11.88 g, 46 mmol) in acetone (400 mL) is added 5-tert-butoxycarbonylamino-4-oxo-tetrahydro-thiopyran-3-carboxylic acid allyl ester (example 1b)) (14.02 g, 44 mmol) and pulverized K₂CO₃ (18.4 g, 132 mmol). The reaction mixture is stirred for 2.5 h at 25-30° C., filtered and evaporated. The residual oil is taken up in EtOAc, washed with brine, dried over MgSO₄, decolorized with charcoal, filtered and evaporated to provide the title compound as a orange oil suitable for use in the next step: TLC (hexane-EtOAc 3:1) Rf=0.25; HPLC Rt_{A}=2.36 min; ESIMS [M+NH3+H]⁺=504.

### e) [(3R*,5S*)-5-(3,5-Difluoro-4-nitro-benzyl)-4-oxo-tetrahydro-thiopyran-3-yl]-carbamic acid tert-butyl ester

To a degassed solution of 5-tert-butoxycarbonylamino-3-(3,5-difluoro-4-nitro-benzyl)-4-oxo-tetrahydro-thiopyran-3-carboxylic acid allyl ester (30.4 g, 62.4 mmol) in THF (300 mL) is added under Argon 5,5-dimethyl-cyclohexane-1,3-dione (11.6 g, 81.2 mmol) and Pd(PPh₃)₄ (0.76 g, 0.62 mmol) and the reaction mixture is stirred for 3 h at 25° C. The reaction mixture is poured into NaH₂PO₄ solution, concentrated and extracted with EtOAc. Combined organic extracts are washed with brine, dried over MgSO₄, filtered and evaporated. The crystallized product containing the(3R*,5S*)-diastereoisomer is filtered off and dried. The mother liquor containing a larger amount of the (3S*,5S*)-diastereoisomer is dissolved in THF and equilibrated with a catalytic amount of DBU to the (3R*,5S*)-diastereoisomer for 16 h at 25° C. After removal of the THF the (3R*,5S*)-diastereoisomer is crystallized from Et₂O-hexane to provide more of the title compound as white crystals: TLC (hexane-EtOAc 3:1) Rf=0.23; HPLC Rt_{A}=2.23 min; ESIMS [M+NH₃+H]⁺=420; ¹H-NMR (400 MHz, CDCl₃): δ 6.96 (d, 2H), 5.65 (d, 1H), 4.55 (m, 1H), 3.40 (m, 1H), 3.25 (dd, 1H), 3.14 (m, 1H), 2.86 (m, 1H), 2.6-2.75 (m, 3H), 1.44 (s, 9H).

### f) [(3R*,4S*,5S*)-5-(3,5-Difluoro-4-nitro-benzyl)-4-hydroxy-tetrahydro-thiopyran-3-yl]-carbamic acid tert-butyl ester

To a suspension of LiAlH₄ (2.137 g, 53.5 mmol) in anhydrous THF (200 mL) is added under Argon a solution of [(3R*,5S*)-5-(3,5-difluoro-4-nitro-benzyl)-4-oxo-tetrahydro-thiopyran-3-yl]-carbamic acid tert-butyl ester (19.56 g, 48.6 mmol) in anhydrous THF (300 mL) below −70° C. over a period of 2 h. After stirring for 5 h at −78° C. the reaction was quenched with 4.2 mL H₂O at 0° C., 4.2 mL 4 N NaOH and after stirring for 30 min an additional 12.6 mL H₂O is added. After addition of MgSO₄, the reaction mixture is filtered over Celite, and the colorless filtrate is evaporated. The title compound is obtained after two crystallizations from THF-EtOAc-diisopropylether as a pure diastereoisomer: TLC (hexane-EtOAc 1:1) Rf=0.38; HPLC Rt_{A}=2.05 min; ESIMS [M+H-isobutylene]⁺=349; ¹H-NMR (400 MHz, CDCl₃): δ6.92 (d, 2H), 5.55 (d, 1H), 4.34 (m, 1H), 3.54 (m, 1H), 3.22 (dd, 1H), 2.91 (m, 1H), 2.75 (m, 1H), 2.46 (dd, 1H), 2.36 (dd, 1H), 2.26 (m, 2H), 2.03 (m, 1H), 1.36 (s, 9H).

### g) [(3R*,4S*,5S*)-5-(3,5-Difluoro-4-nitro-benzyl)-4-hydroxy-1,1-dioxo-hexahydro-1lambda*6*-thiopyran-3-yl]-carbamic acid tert-butyl ester

To a solution of [(3R*,4S*,5S*)-5-(3,5-difluoro-4-nitro-benzyl)-4-hydroxy-tetrahydro-thiopyran-3-yl]-carbamic acid tert-butyl ester (2.05 g, 4.9 mmol) in THF-water 1:1 (60 mL) is added Oxone (6.52 g, 10.3 mmol). After stirring the reaction mixture for 2 h at 40° C., NaOAc (2 g) and sodium metabisulfite (2 g) are added. The reaction mixture is stirred for 0.5 h, basified with saturated K₂CO₃-solution and the product is extracted with EtOAc. Combined organic extracts are washed with brine, dried over MgSO₄, filtered and concentrated to provide the title compound after crystallization from THF-hexane as yellow crystals: TLC (hexane-THF 1:1) Rf=0.23; HPLC Rt_{A}=1.76 min; ESIMS [M+NH3+H]⁺=454; ¹H-NMR (600 MHz, DMSO-d₆): δ 7.32 (d, 2H), 6.91 (d, 1H), 3.73 (m, 1H), 3.0-3.2 (m, 5H), 2.94 (m, 1H), 2.74 (dd, 1H), 2.18 (m, 1H), 1.38 (s, 9H).

### h) (3R*,4S*,5S*)-3-Amino-5-(3,5-difluoro-4-nitro-benzyl)-1,1-dioxo-hexahydro-1lambda*6*-thiopyran-4-ol hydrochloride

To [(3R*,4S*,5S*)-5-(3,5-aifluoro-4-nitro-benzyl )-4-hydroxy-1,1-dioxo-hexahydro-1lambda*6*-thiopyran-3-yl]-carbamic acid tert-butyl ester (0.873 g, 2 mmol) is added 4N HCl in dioxane (5 mL) and the reaction mixture is stirred for 3 h at 40° C. After evaporation the residue is stirred with Et₂O, filtered and dried to provide the title compound as a beige solid: TLC (CH₂Cl₂-MeOH—AcOH—H₂O 180:20:2:1) Rf=0.13; HPLC Rt_{A}=1.22 min; ESIMS [M+H]⁺=337; ¹H-NMR (600 MHz, DMSO-d₆): δ 8.34 (s, 3H), 7.37 (d, 2H), 6.14 (d, 1H), 3.3-3.5 (m, 4H), 3.31 (d, 1H), 3.22 (d, 1H), 3.12 (m, 1H), 2.74 (dd, 1H), 2.26 (m, 1H).

### i) (3R*,4S*,5S*)-3-(3-tert-Butyl-benzylamino)-5-(3,5-difluoro-4-nitro-benzyl)-1,1-dioxo-hexahydro-1lambda*6*-thiopyran-4-ol

The title compound is prepared in analogous manner as described for example 1h) from (3R*,4S*,5S*)-3-amino-5-(3,5-difluoro-4-nitro-benzyl)-1,1-dioxo-hexahydro-1lambda*6*-thio-pyran-4-ol hydrochloride to yield the title compound as a white solid: TLC (EtOAc) Rf=0.27; HPLC Rt_{A}=1.90 min; ESIMS [M+H]⁺=483; ¹H-NMR (400 MHz, CD₃OD): δ 7.41 (m, 1H), 7.1-7.35 (m, 5H), 3.88 (d, 1H), 3.72 (d, 1H), 3.42 (m, 1H), 3.26 (dd, 1H), 3.21 (d, 1H), 2.9-3.1 (m, 3H), 2.85 (m, 1H), 2.72 (dd, 1H), 2.31(m, 1H), 1.34 (s, 9H).

### j) (3R*,4S*,5S*)-3-(3-tert-Butyl-benzylamino)-5-(3-ethoxy-5-fluoro-4-nitro-benzyl)-1,1-dioxo-hexahydro-1lambda*6*-thiopyran-4-ol

To a suspension of (3R*,4S*,5S*)-3-(3-tert-butyl-benzylamino)-5-(3,5-difluoro-4-nitro-benzyl)-1,1-dioxo-hexahydro-1lambda*6*-thiopyran-4-ol (0.30 g, 0.61 mmol) in THF (3 mL) is added EtOH (1.5 mL) and pulverized KOH (0.038 g, 0.65 mmol) and the resulting reaction mixture is heated in the microwave for 20 min at 90° C. The solvent is removed under reduced pressure and the title compound is obtained as a yellow foam suitable for use in the next step: TLC (EtOAc) Rf=0.18; HPLC Rt_{A}=1.98 min; ESIMS [M+H]⁺=509; ¹H-NMR (400 MHz, CDCl₃): δ 7.3 (m, 3H), 7.12 (d, 1H), 6.64 (m, 2H), 4.11 (m, 3H), 3.90 (dd, 1H), 3.76 (dd, 1H), 3.41 (m, 1H), 2.6-3.2 (m, 6H), 2.41 (m, 1H), 1.41 (t, 3H), 1.29 (s, 9H).

### k) (3S*,4S*,5R*)-3-(4-Amino-3-ethoxy-5-fluoro-benzyl)-5-(3-tert-butyl-benzylamino)-1,1-dioxo-hexahydro-1lambda*6*-thiopyran-4-ol dihydrochloride

The title compound is prepared in analogous manner as described for example 1i) from (3R*,4S*,5S*)-3-(3-tert-butyl-benzylamino)-5-(3-ethoxy-5-fluoro-4-nitro-benzyl)-1,1-dioxo-hexahydro-1lambda*6*-thiopyran-4-ol to yield the title compound as a white hydrochloride salt after crystallization from ACN-MeOH-Et₂O: TLC (CH₂Cl₂-MeOH 19:1) Rf=0.31; HPLC Rt_{A}=1.56 min; ESIMS [M+H]⁺=479; ¹H-NMR (600 MHz, DMSO-d₆): δ7.68 (s, 1H), 7.43 1H), 7.39 (d, 1H), 7.36 (t, 1H), 6.59 (m, 2H), 4.23 (m, 2H), 4.05 (m, 2H), 3.87 (dm, 1H), 3.7 (m, 2H), 3.15 (m, 2H), 3.03 (dd, 1H), 2.79 (dm, 1H), 2.40 (dd, 1H), 2.03 (m, 1H), 1.33 (t, 3H), 1.28 (s, 9H).

### Examples 28a -28k

The compounds listed in Table 6 can be prepared by a procedure analogous to that used in example 28.

TABLE 6

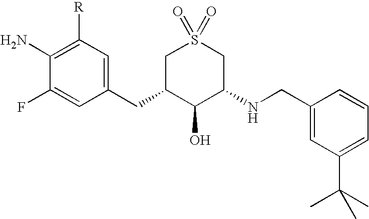

| | | HPLC | HPLC | ESIMS |
|---|---|---|---|---|
| Example | R | Method | Rt [min] | [M + H]⁺ |
| 28a | OCH₂CH₂CH₃ | A | 1.73 | 493 |
| 28b | OCH₂CH₂CH₂CH₃ | A | 1.83 | 507 |
| 28c | OCH(CH₃)₂ | A | 1.67 | 493 |
| 28d | OCH₂CH₂CH₂F | A | 1.62 | 511 |
| 28e | OCH₂CH₂F | A | 1.64 | 497 |
| 28f | OCH₂CH₂CH₃ | A | 1.81 | 547 |
| 28g | 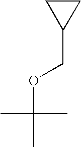 | A | 1.71 | 505 |
| 28h | 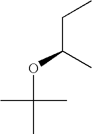 | A | 1.60 | 507 |
| 28i | 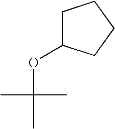 | A | 1.77 | 519 |
| 28j | 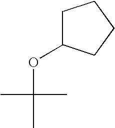 | A | 1.61 | 521 |
| 28k | 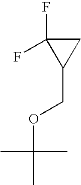 | A | 1.80 | 541 |

### Example 29

### (3S,4S,5R)-3-[4-Amino-3-fluoro-5-(2,2,2-trifluoro-ethoxy)-benzyl]-5-(3-tert-butyl-benzylamino)-1,1-dioxo-hexahydro-1lambda*6*-thiopyran-4-ol dihydrochloride

### a) [(3R,4S,5S)-5-(3,5-Difluoro-4-nitro-benzyl)-4-hydroxy-tetrahydro-thiopyran-3-yl]-carbamic acid tert-butyl ester

The racemate [(3R*,4S*,5S*)-5-(3,5-difluoro-4-nitro-benzyl)-4-hydroxy-tetrahydro-thiopyran-3-yl]-carbamic acid tert-butyl ester (example 28f)) is separated by preparative HPLC on Chiralpak AD-I (5×20 cm) with hexane-AcOEt-iPrOH 80:15:5 to yield the title compound with >99% ee (peak 1) and the (3S,4R,5R)-diastereoisomer with >98% ee as light yellow crystalline solid.

### b) [(3R,4S,5S)-5-(3,5-Difluoro-4-nitro-benzyl)-4-hydroxy-1,1-dioxo-hexahydro-1lambda*6*-thiopyran-3-yl]-carbamic acid tert-butyl ester

The title compound is prepared in analogous manner as described for example 28g) from [(3R,4S,5S)-5-(3,5-difluoro-4-nitro-benzyl )-4-hydroxy-tetrahydro-thiopyran-3-yl]-carbamic acid tert-butyl ester: TLC (hexane-THF 1:1) Rf=0.29; HPLC Rt_{A}=1.78 min; ESIMS [M+NH3+H]⁺454; ¹H-NMR (600 MHz, DMSO-d₆): δ 7.32 (d, 2H), 6.91 (d, 1H), 3.73 (m, 1H), 3.0-3.2 (m, 5H), 2.94 (m, 1H), 2.74 (dd, 1H), 2.18 (m, 1H), 1.38 (s, 9H).

### c) {(3R,4S,5S)-5-[3-Fluoro-4-nitro-5-(2,2,2-trifluoro-ethoxy)-benzyl]-4-hydroxy-1,1-dioxo-hexahydro-1lambda*6*-thiopyran-3-yl}-carbamic acid tert-butyl ester

The title compound is prepared in analogous manner as described for example 28j) from [(3R,4S,5S)-5-(3,5-difluoro-4-nitro-benzyl )-4-hydroxy-tetrahydro-thiopyran-3-yl]-carbamic acid tert-butyl ester and 3,33-trifluoroethanol to yield the title compound after crystallization from THF-Et₂O-hexane as light yellow crystals: TLC (hexane-AcOEt 1:1) Rf=0.10; HPLC Rt_{A}=2.05 min; ESIMS [M+NH3+H]⁺=534.

### d) (3R,4S,5S)-3-Amino-5-[3-fluoro-4-nitro-5-(2,2,2-trifluoro-ethoxy)-benzyl]-1,1-dioxo-hexahydro-1lambda*6*-thiopyran-4-ol hydrochloride

The title compound is prepared in analogous manner as described for example 28h) from {(3R,4S,5S)-5-[3-fluoro-4-nitro-5-(2,2,2-trifluoro-ethoxy)-benzyl]-4-hydroxy-1,1-dioxo-hexa-hydro-1lambda*6*-thiopyran-3-yl}-carbamic acid tert-butyl ester to yield the title compound after evaporation as a light yellow solid: TLC (CH₂Cl₂-MeOH—AcOH—H₂O 180:20:2:1) Rf=0.25; HPLC Rt_{A}=1.58 min; ESIMS [M+H]⁺=417.

### e) (3R,4S,5S)-3-(3-tert-Butyl-benzylamino)-5-[3-fluoro-4-nitro-5-(2,2,2-trifluoro-ethoxy)-benzyl]-1,1-dioxo-hexahydro-1lambda*6*-thiopyran-4-ol

The title compound is prepared in analogous manner as described for example 1h) from (3R,4S,5S)-3-amino-5-[3-fluoro-4-nitro-5-(2,2,2-trifluoro-ethoxy)-benzyl]-1,1-dioxo-hexa-hydro-1lambda*6*-thiopyran-4-ol hydrochloride to yield the title compound after purification by flash-chromatography on silicagel (hexane-EtOAc 1:2 to EtOAc) as a light yellow foam: TLC (EtOAc) Rf=0.31; HPLC Rt_{A}=2.08 min; ESIMS [M+H]⁺=563; ¹H-NMR (400 MHz, CDCl₃): δ 7.3 (m, 3H), 7.14 (d, 1H), 6.92 (d, 1H), 6.75 (s, 1H), 4.46 (m, 2H), 4.15 (s, 1H), 3.92 (d, 1H), 3.75 (d, 1H), 3.41 (m, 1H), 2.6-3.2 (m, 7H), 2.41 (m, 1H), 1.33 (s, 9H).

### f) (3S,4S,5R)-3-[4-Amino-3-fluoro-5-(2,2,2-trifluoro-ethoxy)-benzyl]-5-(3-tert-butyl-benzylamino)-1,1-dioxo-hexahydro-1lambda*6*-thiopyran-4-ol dihydrochloride

The title compound is prepared in analogous manner as described for example 1i) from (3R,4S,5S)-3-(3-tert-butyl-benzylamino)-5-[3-fluoro-4-nitro-5-(2,2,2-trifluoro-ethoxy)-benzyl]-1,1-dioxo-hexahydro-1lambda*6*-thiopyran-4-ol and is obtained as a white solid: TLC (CH₂Cl₂-MeOH 19:1) Rf=0.43; HPLC Rt_{A}=1.87 min; ESIMS [M+H]⁺=533; ¹H-NMR of free base (400 MHz, CDCl₃): δ7.3 (m, 3H), 7.12 (d, 1H), 6.58 (d, 1H), 6.43 (s, 1H), 4.36 (m, 2H), 4.06 (s, 1H), 3.89 (d, 1H), 3.75 (d, 1H), 3.40 (dt, 1H), 2.6-3.2 (m, 7H), 2.32 (m, 1H), 1.35 (s, 9H)

### Examples 30a -30i

The compounds listed in Table 7 can be prepared by a procedure analogous to that used in example 29.

TABLE 7

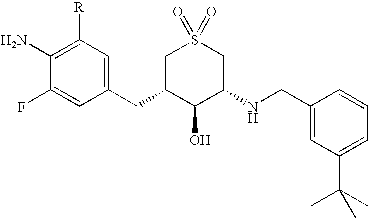

| | | HPLC | HPLC | ESIMS |
|---|---|---|---|---|
| Example | R | Method | Rt [min] | [M + H]⁺ |
| 30a | 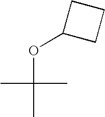 | A | 1.72 | 505 |
| 30b | 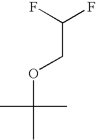 | A | 1.78 | 515 |
| 30c | 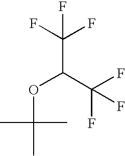 | A | 2.04 | 601 |
| 30d | 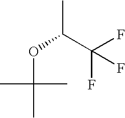 | A | 1.90 | 547 |
| 30e | 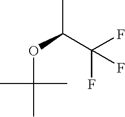 | A | 1.92 | 547 |
| 30f | 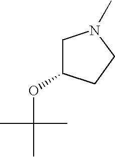 | A | 1.50 | 534 |
| 30g | 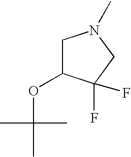 | A | 1.46 | 570 |
| 30h | 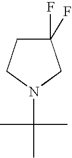 | A | 1.48 | 504 |
| 30i | 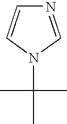 | A | 1.46 | 501 |

### Example 31

### (3S*,4S*,5R*)-3-(4-Amino-3-ethoxy-5-fluoro-benzyl)-5-(5-tert-butyl-2-fluoro-benzylamino)-1,1-dioxo-hexahydro-1lambda*6*-thiopyran-4-ol dihydrochloride

### a) [(3R*,4S*,5S*)-5-(3-Ethoxy-5-fluoro-4-nitro-benzyl)-4-hydroxy-1,1-dioxo-hexahydro-1lambda*6*-thiopyran-3-yl]-carbamic acid tert-butyl ester

To a suspension of [(3R*,4S*,5S*)-5-(3,5-difluoro-4-nitro-benzyl)-4-hydroxy-1,1-dioxo-hexa-hydro-1lambda*6*-thiopyran-3-yl]-carbamic acid tert-butyl ester (example 28g)) (1.7 g, 3.8 mmol) in THF (15 mL) is added EtOH (4 mL) and pulverized KOH (0.229 g, 4.0 mmol) and the resulting reaction mixture is heated in the microwave for 30 min at 90° C. and 10 min at 95° C. The solvent is removed under reduced pressure and the title compound is obtained as a yellow solid suitable for use in the next step: TLC (EtOAc) Rf=0.14; HPLC Rt_{A}=1.91 min; ESIMS [M+H]⁺=480; ¹H-NMR (400 MHz, CDCl₃+5% CD₃OD): 6.62 (m, 2H), 4.14 (m, 3H), 3.93 (m, 1H), 2.3-3.4 (m, 7H), 1.41 (s, 9H), 1.39 (t, 3H).

### b) (3R*,4S*,5S*)-3-Amino-5-(3-ethoxy-5-fluoro-4-nitro-benzyl)-1,1-dioxo-hexahydro-1lambda*6*-thiopyran-4-ol hydrochloride

The title compound is prepared in analogous manner as described for example 1g) from [(3R*,4S*,5S*)-5-(3-ethoxy-5-fluoro-4-nitro-benzyl)-4-hydroxy-1,1-dioxo-hexahydro-1lambda*6*-thiopyran-3-yl]-carbamic acid tert-butyl ester to yield the title compound as a light yellow hydrochloride salt after crystallization from iPrOH: TLC (CH₂Cl₂-MeOH—AcOH—H₂O 180:20:2:1) Rf=0.18; HPLC Rt_{A}=1.46 min; ESIMS [M+H]⁺=363; ¹H-NMR (400 MHz, CD₃OD): δ 6.95 (s, 1H), 6.84 (d, 1H), 4.22 (q, 2H), 2.8-3.6 (m, 7H), 2.64 (dd, 1H), 2.39 (m, 1H), 1.38 (t, 3H).

### c) (3R*,4S*,5S*)-3-(5-tert-Butyl-2-fluoro-benzylamino)-5-(3-ethoxy-5-fluoro-4-nitro-benzyl)-1,1-dioxo-hexahydro-1lambda*6*-thiopyran-4-ol

The title compound is prepared in analogous manner as described for example 1h) from (3R*,4S*,5S*)-3-amino-5-(3-ethoxy-5-fluoro-4-nitro-benzyl)-1,1-dioxo-hexahydro-1lambda*6*-thiopyran-4-ol hydrochloride and 5-tert-butyl-2-fluoro-benzaldehyde to yield the title compound after purification by flash-chromatography on silicagel (hexane-THF 2:1 THF) as a light yellow foam: TLC (toluene-THF 1:1) Rf=0.32; HPLC Rt_{A}=1.99 min; ESIMS [M+H]⁺=527.

### d) (3S*,4S*,5R*)-3-(4-Amino-3-ethoxy-5-fluoro-benzyl)-5-(5-tert-butyl-2-fluoro-benzyl-amino)-1,1-dioxo-hexahydro-1lambda*6*-thiopyran-4-ol dihydrochloride

The title compound is prepared in analogous manner as described for example 1i) from (3R*,4S*,5S*)-3-(5-tert-butyl-2-fluoro-benzylamino)-5-(3-ethoxy-5-fluoro-4-nitro-benzyl)-1,1-dioxo-hexahydro-1lambda*6*-thiopyran-4-ol and is obtained as a white solid: TLC (toluene-THF 1:1) Rf=0.27 HPLC Rt_{A}=1.58 min; ESIMS [M+H]⁺=497; ¹H-NMR (600 MHz, DMSO-d₆): δ 9.98 (s, 1H), 9.29 (s, 1H), 7.80 (m, 1H), 7.48 (m, 1H), 7.20 (t, 1H), 6.68 (m, 2H), 6.25 (s, 1H), 4.3 (s, 2H), 4.09 (m, 2H), 3.74 (m, 3H), 3.36 (m, 1H), 3.16 (dd, 1H), 3.06 (dd, 1H), 2.85 (d, 1H), 2.46 (d, 1H), 2.12 (m, 1H), 1.35 (t, 3H), 1.29 (s, 9H).

### e) 5-tert-Butyl-2-fluoro-benzaldehyde

The title compound is prepared in analogous manner as described for the des-fluoro derivative in Organic & Biomolecular Chemistry (2007), 5(23), 3778, starting from 2-bromo-4-tert-butyl-1-fluoro-benzene and is obtained as a light yellow oil: TLC (hexane-EtOAc 10:1) Rf=0.48; HPLC Rt_{A}=2.10 min; ¹H-NMR (400 MHz, CDCl₃): δ 10.35 (s, 1H), 7.86 (dd, 1H), 7.63 (m, 1H), 7.09 (t, 1H), 1.32 (s, 9H).

### Examples 31a -31o

The compounds listed in Table 8 can be prepared by a procedure analogous to that used in example 31.

TABLE 8

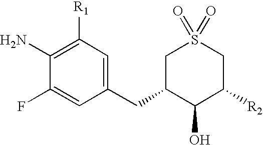

| | | | HPLC | HPLC | ESIMS |
|---|---|---|---|---|---|
| Example | R₁ | R₂ | Method | Rt [min] | [M + H]⁺ |
| 31a | 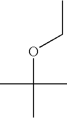 | 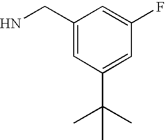 | A | 1.62 | 497 |
| 31b | 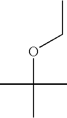 | 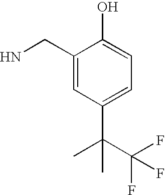 | A | 1.54 | 549 |
| 31c | 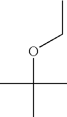 | 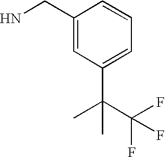 | A | 1.60 | 533 |
| 31d | 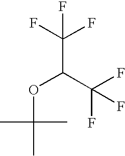 | 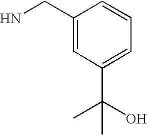 | A | 1.76 | 603 |
| 31e | 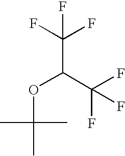 | 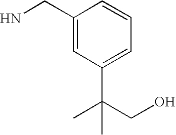 | A | 1.84 | 617 |
| 31f | 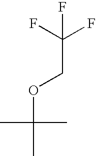 | 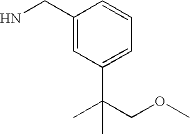 | C | 3.80 | 563 |
| 31g | 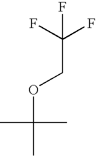 | 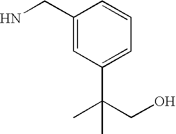 | C | 3.36 | 549 |
| 31h | 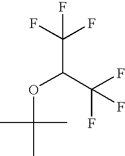 | 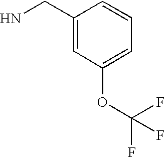 | H | 1.08 | 629 |
| 31i | 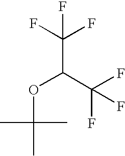 | 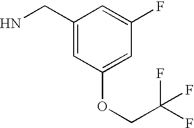 | I | 1.24 | 661 |
| 31j | 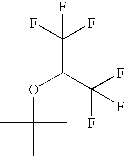 | 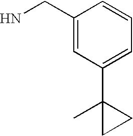 | H | 1.07 | 599 |
| 31k | 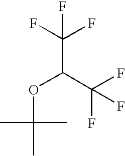 | 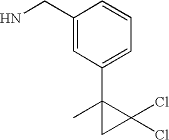 | H | 1.17 | 667, 669 |
| 31l | 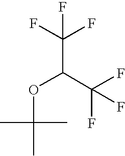 | 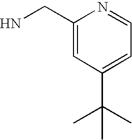 | H | 1.09 | 602 |
| 31m | 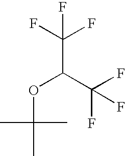 | 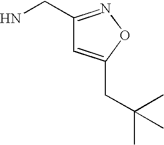 | H | 1.19 | 606 |
| 31n | 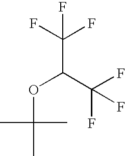 | 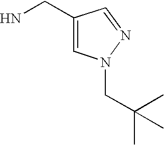 | H | 1.04 | 605 |
| 31o | 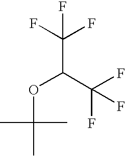 | 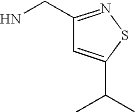 | J | 2.71 | 594 |

### 3-tert-Butyl-5-fluoro-benzaldehyde (Example 31 a) can be Synthesized using the Following Procedure

### a) 2-Bromo-4-tert-butyl-6-fluoro-phenylamine

To a solution of 4-tert-butyl-2-fluoro-phenylamine (1.037 g, 6.2 mmol) in ACN-water 2:1 (40 mL) is added NaBr (0.646 g, 6.2 mmol) and oxone (3.86 g, 6.2 mmol) in small portions over a period of 0.5 h at 25° C. The reaction mixture is stirred for 1.5 h at 25° C. before it is added to a 10% solution of Na₂S₂O₃ solution. After basification with NaHCO₃ the product is extracted with EtOAc. Combined organic layers are washed with brine, dried over MgSO₄, filtered and evaporated. The title compound is obtained after purification by chromatography (CombiFlash, 40 g silica gel, hexane-EtOAc 10:1 to EtOAc) as a brownish oil: TLC (hexane-EtOAc 10:1) Rf=0.42; HPLC Rt_{A}=2.24 min; ¹H-NMR (400 MHz, CDCl₃): δ 7.20 (s, 1H), 6.98 (d, 1H), 3.96 (s, 2H), 1.25 (s, 9H).

### b) 1-Bromo-3-tert-butyl-5-fluoro-benzene

To a solution of tert-butylnitrit (0.792 g, 1.5 mmol) in DMF (10 mL) is slowly added a solution of 2-bromo-4-tert-butyl-6-fluoro-phenylamine (1.23 g, 5.0 mmol) dissolved in DMF (10 mL).

After stirring for 4 h at 60° C. bortrifluoride etherate 0.70 mL, 5.0 mmol) is added at 25° C. and the reaction mixture is stirred for 0.5 h at 25° C. The reaction mixture is poured onto ice-water and extracted with Et₂O. Combined organic layers are washed with brine, dried over MgSO₄, filtered and evaporated. The title compound is obtained after purification by chromatography (CombiFlash, 40 g silica gel, hexane to hexane-EtOAc 1:1) as a colorless oil: TLC (hexane) Rf=0.51; HPLC Rt_{A}=2.53 min; ¹H-NMR (400 MHz, CDCl₃): δ 7.27 (s, 1H), 7.06 (d, 1H), 7.01 (d, 1H), 1.27 (s, 9H).

### c) 3-tert-Butyl-5-fluoro-benzaldehyde

To a solution of 1-bromo-3-tert-butyl-5-fluoro-benzene (1.04 g, 4.5 mmol) in anhydrous THF is added under Argon at −78° C. 2.5 M nBuLi in hexane (1.9 mL, 4.7 mmol) and after stirring for 0.5 h at −78° C. DMF (0.70 mL, 9 mmol) is added. After stirring for 1.5 h at −78° C. the reaction mixture is added to 0.5 N aqueous HCl and extracted with Et₂O. Combined organic layers are washed with brine, dried over MgSO₄, filtered and evaporated. The title compound is obtained as a light yellow oil and is used a s such for the next transformation: TLC (hexane-EtOAc 10:1) Rf=0.36; HPLC Rt_{A}=2.08 min; 1H-NMR (400 MHz, CDCl3): δ 9.98 (s, 1H), 7.68 (s, 1H), 7.36 (m, 1H), 1.25 (s, 9H).

### 2-Hydroxy-5-(2,2,2-trifluoro-1,1-dimethyl-ethyl)benzaldehyde (Example 31b) can be Synthesized using the Following Procedure

To a mixture of 2-methoxy-5-(2,2,2-trifluoro-1,1-dimethyl-ethyl)benzaldehyde (6.7 g, 27.2 mmol) is added dropwise an 1M CH₂Cl₂ solution of boron tribromide (130 mL, 130 mmol) keeping the temperature below 30° C. The mixture is stirred at room temperature for 21 h. The reaction is quenched with ice water and extracted with EtOAc. The combined organic phases are washed with water and brine, dried over sodium sulfate, filtered and concentrated. The crude product is purified by column chromatography on silica gel (hexane-EtOAc 19:1) to furnish title compound as a colorless oil: HPLC Rt_{E}=7.43 min; ESIMS [M+H]⁺=231; ¹H-NMR (400 MHz, CDCl₃): δ 10.99 (s, 1H), 9.91 (s, 1H), 7.66 (dd, 2H), 7.0 (d, 1H), 1.59 (m, 6H), ¹⁹F-NMR (400 MHz, CDCl₃): δ−77.0.

### 3-(2,2,2-Trifluoro-1,1-dimethyl-ethyl)benzaldehyde (example 31c) can be Synthesized Using the Following Procedure

### a) 2-trifluoromethanesulfonyloxy-5-(2,2,2-trifluoro-1,1-dimethyl-ethyl)benzaldehyde

To an ice cold solution of 2-hydroxy-5-(2,2,2-trifluoro-1,1-dimethyl-ethyl)benzaldehyde (7.07 g, 30.4 mmol) and pyridine (4.9 mL, 61 mmol) in CH₂Cl₂ (50 mL) is added dropwise triflic anhydride (7.084 mL, 45.7 mmol) keeping the temperature below 10° C. and the mixture is stirred at 0° C. for 45 min. The reaction mixture is quenched with NaHCO₃ and ice and the organic layer is separated. The aqueous layer is extracted with Et₂O and the combined organic layers are washed with water, brine, dried over sodium sulfate, filtered and concentrated to give the title compound after purification by column chromatography on silica gel (hexane-acetone 19:1) as a white solid: HPLC Rt_{E}=7.75 min; ESIMS [M+H]⁺=365; ¹H-NMR (400 MHz, CDCl₃): δ 10.28 (s, 1H), 8.10 (d, 1H), 7.85 (dd, 1H), 7.42 (d, 1H), 1.63 (m, 6H), ¹⁹F-NMR (400 MHz, CDCl₃): δ−73.2, −76.6.

### b) 3-(2,2,2-trifluoro-1,1-dimethyl-ethyl)benzaldehyde

A mixture of 2-trifluoromethanesulfonyloxy-5-(2,2,2-trifluoro-1,1-dimethyl-ethyl)benzaldehyde (8.24 g, 22.6 mmol), 10% Pd/C (0.82 g) and diethylamine (2.8 mL, 27.1 mmol) in MeOH (50 mL) is stirred at 25° C. under hydrogen pressure from 10 to 3.7 bar within 25 min. The reaction mixture is filtered over Celite and the filtrate is concentrated. The crude product is purified by column chromatography on silica gel (hexane-EtOAc 95:5) to provide the title compound as a light yellow oil: HPLC Rt_{E}=7.39 min, ¹H-NMR (400 MHz, CDCl₃): δ 10.04 (s, 1H), 8.01 (s, 1H), 7.85 (dd, 1H), 7.83 (dd, 1H), 7.55 (t, 1H) 1.63 (m, 6H), ¹⁹F-NMR (400 MHz, CDCl₃): δ−76.6.

### 3-(2-Methoxy-1,1-dimethyl-ethyl)-benzaldehyde (example 31f) can be Synthesized Using the Following Procedure

### a) 2-(3-[1,3]Dioxolan-2-yl-phenyl)-2-methyl-propionic acid methyl ester

To a solution of dicyclohexylamine (3.95 mL, 19.8 mmol) in toluene at −20° C. under argon is added dropwise a 1.6 N solution of nBuLi in hexane (12.4 mL, 19.8 mmol). After 15 min at 0° C., methyl isobutyrate (1.75 g, 17.2 mmol) is added dropwise to the reaction mixture, which is allowed to warm to 25° C. and stirred for 15 min at 25° C. Then, 2-(3-bromophenyl)-1,3-dioxolane (2 mL, 13.2 mmol), Pd₂(dba)₃ (0.152 g, 0.264 mmol) and P(tBu)₃ (0.02 eq, 63 ul) are added and the reaction mixture is stirred for 1 h. The reaction mixture is quenched with 1 N HCl in Et₂O to precipitate the dicyclohexylamine as HCl salt. The reaction mixture is filtered and concentrated. The title compound is obtained after purification by flash-chromatography on silica gel (cyclohexane-EtOAc 1:0 to 4:1): HPLC Rt_{c}=3.46 min; ESIMS [M+H]⁺=251. ¹H-NMR (400 MHz, CDCl₃): δ 7.4 (s, 1H), 7.25 (d, 3H), 5.8 (s, 1H), 4.1 (t, 2H), 4.0 (t, 2H), 3.8 (s, 3H), 1.55 (s, 6H).

### b) 2-(3-[1,3]Dioxolan-2-yl-phenyl)-2-methyl-propan-1-ol

To a solution of LiAlH₄ (0.042 g, 1.10 mmol) in Et₂O (1.1 ml) is added dropwise a solution of 2-(3-[1,3]dioxolan-2-yl-phenyl)-2-methyl-propionic acid methyl ester (0.250 g, 1 mmol) dissolved in Et₂O (6 ml). The reaction mixture is stirred for 30 min at 25° C. The reaction mixture is quenched with saturated aq. potassium sodium tartrate and was filtered through a pad of Celite. The organic layer is washed with brine, dried over Na₂SO₄, filtered and concentrated. The rrude title compound is used as such in the next reaction. LC-MS Rt_{c}=2.76 min; ESIMS [M+H]⁺=223.

### c) 2-[3-(2-Methoxy-1,1-dimethyl-ethyl)-phenyl]-[1,3]dioxolane

To a solution of 2-(3-[1,3]dioxolan-2-yl-phenyl)-2-methyl-propan-1-ol (0.250 mg, 1 mmol) in THF (10 mL) at 25° C. under argon is added NaH (0.121 mg, 3.04 mmol). After 10 min stirring at 25° C., MeI (0.193 mL, 3.04 mmol) is added and solution is stirred at 80° C. for 1 h. The reaction mixture is extracted with EtOAc. The organic layers are washed with water and brine, dried over Na₂SO₄, filtered and concentrated. The title compound is obtained after purification by flash-chromatography on silica gel (cyclohexane-EtOAc 1:0 to 4:1): LC-MS Rt_{c}=4.76 min; ESIMS [M+H]⁺=237. ¹H-NMR (400 MHz, CDCl₃): δ 7.5 (s, 1H), 7.4 (m, 1H), 7.25 (d, 2H), 5.8 (s, 1H), 4.15 (m, 2H), 4.0 (m, 2H), 3.4 (s, 2H), 3.3 (s, 3H), 1.55 (s, 6H).

### d) 3-(2-Methoxy-1,1-dimethyl-ethyl)-benzaldehyde

To a solution of 2-[3-(2-methoxy-1,1-dimethyl-ethyl)-phenyl]-[1,3]dioxolane (2.125 g, 9 mmol) in THF (40 mL) is added 4 N aq. HCl (11.2 mL, 45 mmol). The reaction mixture is stirred at 75° C. for 30 min. The reaction mixture is extracted with EtOAc, washed with brine, dried over Na₂SO₄, filtered and concentrated. The title compound obtained as a light yellow oil is used as such in next reaction. LC-MS Rt_{c}=3.44 min. ¹H-NMR (400 MHz, CDCl₃): δ 10.0 (s, 1H), 7.9 (s, 1H), 7.7(dd, 2H), 7. 5 (t, 1H), 3.4 (s, 2H), 3.3 (s, 3H), 1.35 (s, 6H).

### 3-Fluoro-5-(2,2,2-trifluoroethoxy)-benzaldehyde (example 31 i) can be Synthesized Using the Following Procedure

### a) 1-Bromo-3-fluoro-5-(2,2,2-trifluoroethoxy)-benzene

To a solution of 1-bromo-3,5-difluoro-benzene (4.83 g, 25 mmol) and 2,2,2,-trifluoroethanol (2.72 mL, 37.5 mmol) in DMSO (5 mL) is added a solution of potassium tert. butoxide (3.18 g, 27.5 mmol) in DMSO (30 mL) while maintaining the temperature of the reaction mixture below 25° C., stirring is continued at room temperature for 1 h. The reaction mixture is quenched with ice, diluted with water and extracted with toluene. The combined organic layers are washed with water dried over Na₂SO₄, filtered and concentrated to give the title compound as a colorless oil: TLC (cyclohexane-EtOAc 50:50) Rf=0.69; ESIMS [M+H]⁺=273; ¹H-NMR (400 MHz, DMSO-d₆): δ 7.21 (d, 1H), 7.18 (s, 1H), 7.05 (d, 1H), 4.83 (q, 2H).

### b) 3-Fluoro-5-(2,2,2-trifluoroethoxy)-benzaldehyde

To a solution of 1-bromo-3-fluoro-5-(2,2,2-trifluoroethoxy)-benzene (6.83 g, 25 mmol) in Et₂O (50 mL) is added dropwise at −78° C. 1.1M n-BuLi (22.7 mL, 25 mmol) the mixture is stirred for 15 min. DMF (2.13 mL, 27.5 mmol) is added at −78° C. after stirring for 5 min the reaction mixture is quenched with 1M aq. HCl and the mixture is allowed to warm to room temperature. The organic layer is separated and the aqueous layer is extracted with Et₂O. The combined organic layers are washed with water, dried over Na₂SO₄, filtered and concentrated to give the title compound after purification by column chromatography on silica gel (cyclohexane to cyclohexane-EtOAc 50:50) as a yellowish oil: TLC (cyclohexane-EtOAc 80:20) Rf=0.46; ESIMS [M−H]⁻=222; ¹H-NMR (400 MHz, DMSO-d₆): δ 9.92 (s, 1H), 7.45 (s, 1H), 7.40-7.35 (m, 2H), 4.90 (q, 2H).

### 3-(1-Methyl-cyclopropyl)benzaldehyde (example 31j)

The title compound is prepared following a procedure described in J. Am. Chem. Soc. 2007, 127, 12440-12441, starting from 2-(3-isopropenyl-phenyl)-[1,3]dioxolane and is obtained as a light yellow oil: TLC (cyclohexane-EtOAc 80:20) Rf=0.52; HPLC Rtₗ=1.35 min; ¹H-NMR (400 MHz, DMSO-d₆): δ 9.98 (s, 1H), 7.74 (s, 1H), 7.68 (d, 1H), 7.55-7.47 (m, 2H), 1.40 (s, 3H), 0.89 (t, 2H), 0.81 (, 2H).

### 3-(2,2-Dichloro-1-methyl-cyclopropyl)benzaldehyde (example 31k) can be Synthesized Using the Following Procedure

### a) 2-[3-(2,2-Dichloro-1-methyl-cyclopropyl)-phenyl]-[1,3]dioxolane

To a solution of 2-(3-isopropenyl-phenyl)-[1,3]dioxolane (3.8 g, 20 mmol) and CHCl₃ (8.19 mL, 100 mmol) in 50% aq. NaOH (100 mL) and CH₂Cl₂ (100 mL) is added benzyltriethylammoniumchloride (93 mg, 0.4 mmol) and the mixture is vigorously stirred at room temperature for 2 h. The reaction mixture is quenched with ice, diluted with water and CH₂Cl₂ and the organic layer is separated. The aqueous layer is extracted with CH₂Cl₂ and the combined organic layers are dried over Na₂SO₄, filtered and concentrated to give the title compound after purification by column chromatography on silica gel (cyclohexane to cyclohexane-EtOAc 80:20) as a yellow oil: TLC (cyclohexane-EtOAc 80:20) Rf=0.45; HPLC Rt_{H}=1.38 min; ESIMS [M+H]⁺=273; ¹H-NMR (400 MHz, DMSO-d₆): δ 7.39-7.32 (m, 4H), 5.71 (s, 1H), 4.08-4.00 (m, 2H), 3.97-3.89 (m, 2H), 2.13 (d, 1H), 1.77 (d, 1H), 1.60 (s, 3H).

### b) 3-(2,2-Dichloro-1-methyl-cyclopropyl)benzaldehyde

To a solution of 2-[3-(2,2-dichloro-1-methyl-cyclopropyl)-phenyl]-[1,3]dioxolane (1.15 g, 4.2 mmol) are added 1M aq. HCl (30 mL) and conc. H₂SO₄ (50 μL) and the mixture is stirred at room temperature for 2 h. The reaction mixture is extracted with Et₂O, the Et₂O extract is washed with 2M aq. NH₃ and dried over sodium sulfate, filtered and concentrated to provide the title compound as a yellow oil: TLC (cyclohexane-EtOAc 80:20) Rf=0.41; HPLC Rt_{H}=1.32 min; ¹H-NMR (400 MHz, DMSO-d₆): δ 10.02 (s, 1H), 7.89 (s, 1H), 7.82 (d, 1H), 7.71 (d, 1H), 7.60 (t, 1H), 2.24 (d, 1H), 1.84 (d, 1H), 1.65 (s, 3H).

### 5-Isopropyl-isothiazole-3-carbaldehyde (example 31o) can be Synthesized Using the Following Procedure

### a) 3-Furan-2-yl-5-isopropyl-isothiazole

To a solution of 1-amino-1-furan-2-yl-4-methyl-pent-1-en-3-one (8.15 g, 45.5 mmol) in THF (283 mL) is added phosphor pentasulfide (5.05 g, 22.74 mmol) and the mixture is stirred 36 h at 25° C. The reaction mixture is evaporated, taken up in diethyl ether (150 mL) and 150 mL of 30% hydrogen peroxide is added dropwise. After stirring vigorously for 10 min activated charcoal is added and the mixture is filtered through Celite. The filtrate is washed with brine, sodium hydrogen sulfite and brine. The crude product is purified on silica gel (hexane to hexane-tBuOMe 40:1) to yield the title compound as a yellowish liquid: TLC (hexane/EtOAc=3:1) Rf=0.70; ESIMS [M+H]⁺=194; ¹H-NMR (400 MHz, CDCl₃): δ 7.45 (s, 1H), 7.23 (s, 1H), 6.84 (s, 1H), 6.45 (m, 1H), 3.25 (heptet, 1H, J=7), 1.40 (d, 6H, J=7).

### b) 5-Isopropyl-isothiazole-3-carboxylic acid

To a suspension of 3-furan-2-yl-5-isopropyl-isothiazole (5.94 g, 30.7 mmol) in acetone (50 mL) and water (100 mL) is added KMnO₄ (9.70 g, 61.4 mmol) portion wise and the mixture is stirred. The reaction is slightly exothermic and gas develops. After 1.5 h 150 ml 2N NaOH is added and the mixture is briefly heated to 50° C. The mixture is filtered over Celite and the filtrated is washed with tBuOMe. The aqueous phase is acidified with conc. HCl and extracted with EtOAc. The organic phase is dried with MgSO₄ and concentrated to provide the title compound as a brown oil, pure enough for further transformation: HPLC Rt_{J}=1.99 min; ESIMS [M+H]⁺=172; ¹H-NMR (400 MHz, CDCl₃): δ 7.61 (s, 1H), 3.27 (heptet, 1H, J=7), 1.40 (d, 6H, J=7).

### c) (5-Isopropyl-isothiazol-3-yl)-methanol

To a solution of 5-isopropyl-isothiazole-3-carboxylic acid (0.5 g, 2.92 mmol) in THF (4 mL) is added under argon BH₃-Me₂S complex (0.38 mL, 3.8 mmol) at 25° C. The reaction mixture is stirred for 16 h at 25° C. and quenched by the slow addition of MeOH. The reaction mixture is evaporated several times with MeOH. The residual oil is dissolved in tBuOMe, washed with 1 N aq. HCl, 1N aq. NaOH and brine, dried over MgSO₄, filtered and concentrated to yield the title compound as a yellow oil, pure enough for the next transformation: HPLC Rt_{J}=1.93 min; ESIMS [M+H]⁺=158; ¹H-NMR (400 MHz, CDCl₃): δ 6.87 (s, 1H), 4.68 (br, 2H), 3.23 (heptet, 1H, J=7), 1.40 (d, 6H, J=7).

### d) 5-Isopropyl-isothiazole-3-carbaldehyde

To a solution of (5-isopropyl-isothiazol-3-yl)-methanol (0.1 g, 0.584 mmol) in EtOAc (2 mL) is added activated MnO₂ (508 mg, 5.84 mmol) and stirred overnight. The mixture is filtered over Celite and concentrated to yield the title compound as a colorless oil pure enough for further transformation: TLC (hexane-EtOAc=9:1) Rf=0.40; ¹H-NMR (400 MHz, CDCl₃): δ 9.99 (s, 1H), 7.48 (s, 1H), 3.26 (heptet, 1H, J=7), 1.40 (d, 6H, J=7).

### Example 32

### (3S*,4S*,5R*)-3-(4-Amino-3-ethoxy-5-fluoro-benzyl)-5-[(S*)-1-(3-tert-butyl-phenyl)-ethylamino]-1,1-dioxo-hexahydro-1lambda*6*-thiopyran-4-ol dihydrochloride and (3S*,4S*,5R*)-3-(4-Amino-3-ethoxy-5-fluoro-benzyl)-5-[(R*)-1-(3-tert-butyl-phenyl)-ethylamino]-1,1-dioxo-hexahydro-1lambda*6*-thiopyran-4-ol dihydrochloride

### a) (3R*,4S*,5S*)-3-[(S*)-1- and (3R*,4S*,5S*)-3-[(R*)-1-(3-tert-Butyl-phenyl)-ethylamino]-5-(3-ethoxy-5-fluoro-4-nitro-benzyl)-1,1-dioxo-hexahydro-1lambda*6*-thiopyran-4-ol

To a solution of (3R*,4S*,5S*)-3-amino-5-(3-ethoxy-5-fluoro-4-nitro-benzyl)-1,1-dioxo-hexa-hydro-1lambda*6*-thiopyran-4-ol (example 31b) (0.20 g, 0.54 mmol) in isopentylalcohol (5 mL) is added 1-(3-tert-butyl-phenyl)-ethanone (0.29 g, 1.62 mmol) and the reaction mixture is heated at 150° C. for 18 h. To the cooled reaction mixture is added MeOH (5 mL) and NaBH₃CN (0.076 g, 1.08 mmol) and a few drops of AcOH. After stirring for 0.5 h at 25° C., the solution is added to 1N aqueous HCl, stirred for 10 min, basified with solid NaHCO₃ and extracted with EtOAc. Combined organic layers are washed with brine, dried over MgSO₄, filtered and evaporated. The diastereoisomers of the title compound are obtained after purification by chromatography (CombiFlash, 12 g silica gel, hexane-EtOAc 10:1 to EtOAc) as light yellow oils. Diastereoisomer I: TLC (CH₂Cl₂-MeOH 19:1) Rf=0.55; HPLC Rt_{A}=2.05 min; ESIMS [M+H]⁺=523 and Diastereoisomer II: TLC (CH₂Cl₂-MeOH 19:1) Rf=0.40; HPLC Rt_{A}=2.03 min; ESIMS [M+H]⁺=523.

### b) (3R*,4S*,5S*)-3-(4-Amino-3-ethoxy-5-fluoro-benzyl)-5-[(S*)-1-(3-tert-butyl-phenyl)-ethylamino]-1,1-dioxo-hexahydro-1lambda*6*-thiopyran-4-ol dihydrochloride and (3R*,4S*,5S*)-3-(4-Amino-3-ethoxy-5-fluoro-benzyl)-5-[(R*)-1-(3-tert-butyl-phenyl)-ethylamino]-1,1-dioxo-hexahydro-1lambda*6*-thiopyran-4-ol dihydrochloride

The title compounds are prepared in analogous manner as described for example 1i) from the corresponding diastereoisomers of example 32a) and are purified by preparative HPLC (Sun Five C₁₈ OBD 5 μm, 100×30, 5-100% ACN in water+0.1% TFA gradient, 25 min) to yield the hydrochloride salts as white amorphous solids: TLC (hexane-THF 1:1) Rf=0.41 and 0.27; HPLC Rt_{A}=1.62 min and 1.65 min; ESIMS [M+H]⁺=493.

### Example 33

### (1S,3S,4S,5R)-3-[4-Amino-3-fluoro-5-(2,2,2-trifluoro-ethoxy)-benzyl]-5-(3-tert-butyl-benzylamino)-1-oxo-tetrahydro-thiopyran-4-ol (trans-sulfoxide)

### a) [(1S,3R,4S,5S)-5-(3,5-Difluoro-4-nitro-benzyl)-4-hydroxy-1-oxo-tetrahydro-thiopyran-3-yl]-carbamic acid tert-butyl ester

To a solution of [(3R,4S,5S)-5-(3,5-difluoro-4-nitro-benzyl)-4-hydroxy-tetrahydro-thiopyran-3-yl]-carbamic acid tert-butyl ester (example 29a)) (2.0 g, 4.9 mmol) in THF-water 2:1 (150 mL) is added at 0-5° C. oxone in small portions (2.85 g, 4.4 mmol) over a period of 1 h. The oxidation is stopped with sodium metabisulfite (0.5 g) and after stirring for 30 min extracted with EtOAc. Combined organic layers are washed with saturated NaHCO₃-solution and brine, dried over MgSO₄, filtered and concentrated. The crude product is recrystallized from THF-EtOAc-hexane to give the title compound as white crystals: TLC (CH₂Cl₂-MeOH 19:1) Rf=0.21; HPLC Rt_{A}=1.60 min; ESIMS [M+H-isobutylene]⁺=365; ¹H-NMR (400 MHz, CDCl₃+2% CD₃OD): δ 6.97 (d, 2H), 5.76 (s, 1H), 3.76 (m, 1H), 3.42 (m, 1H), 3.34 (m, 1H), 3.23 (dd, 1H), 3.08 (dd, 1H), 2.89 (m, 1H), 2.74 (m, 1H), 2.53 (dd, 1H), 2.05 (m, 1H), 1.42 (t, 9H).

### b) (1S,3R,4S,5S)-3-Amino-5-(3,5-difluoro-4-nitro-benzyl)-1-oxo-tetrahydro-thiopyran-4ol

To a solution of [(1S,3R,4S,5S)-5-(3,5-difluoro-4-nitro-benzyl)-4-hydroxy-1-oxo-tetrahydro-thiopyran-3-yl]-carbamic acid tert-butyl ester (1.55 g, 3.65 mmol) in CH₂Cl₂ (20 mL) is added TFA (3 mL) and the reaction mixture is stirred for 2 h at 25° C. The solvents are removed under reduced pressure and the residual foam is basified with 20% aqueous K₂CO₃ solution and the product extracted with EtOAc. Combined organic layers are washed with brine, dried over MgSO₄, filtered and evaporated to provide the title compound as a white amorphous solid: TLC (CH₂Cl₂-MeOH—AcOH—H₂O 180:20:2:1) Rf=0.04; HPLC Rt_{A}=1.04 min; ESIMS [M+H]+=321.

### c) (1S,3R,4S,5S)-3-(3-tert-Butyl-benzylamino)-5-(3,5-difluoro-4-nitro-benzyl)-1-oxo-tetrahydro-thiopyran-4-ol

The title compound is prepared in an analogous manner as described for example 1h), starting from (1S,3R,4S,5S)-3-amino-5-(3,5-difluoro-4-nitro-benzyl )-1-oxo-tetrahydro-thio-pyran-4-ol and 3-tert-butyl-benzaldehyde to yield a light yellow foam: TLC (CH₂Cl₂-MeOH 19:1) Rf=0.30; HPLC Rt_{A}=1.78 min; ESIMS [M+H]⁺=467; ¹H-NMR (400 MHz, CDCl₃): δ 7.32 (m, 3H), 7.13 (d, 1H), 6.95 (d, 2H), 3.97 (s, 1H), 3.93 (d, 1H), 3.75 (d, 1H), 3.71 (dt, 1H), 3.15 (m, 2H), 2.96 (dd, 1H), 2. 82 (dd, 1H), 2.62 (ddd, 1H), 2.41 (m, 2H), 1.97 (m, 1H), 1.34 (s, 9H).

### d) (1S,3R,4S,5S)-3-(3-tert-Butyl-benzylamino)-5-[3-fluoro-4-nitro-5-(2,2,2-trifluoro-ethoxy)-benzyl]-1-oxo-tetrahydro-thiopyran-4-ol

The title compound is prepared in an analogous manner as described for example 28j), starting from (1S,3R,4S,5S)-3-(3-tert-butyl-benzylamino)-5-(3,5-difluoro-4-nitro-benzyl)-1-oxo-tetrahydro-thiopyran-4-ol and 3,3,3-trifluoroethanol to provide the title compound as a light yellow foam: TLC (CH₂Cl₂-MeOH—AcOH—H₂O 180:20:2:1) Rf=0.47; HPLC Rt_{A}=1.96 min; ESIMS [M+H]⁺=547.

### e) (1S,3S,4S,5R)-3-[4-Amino-3-fluoro-5-(2,2,2-trifluoro-ethoxy)-benzyl]-5-(3-tert-butyl-benzylamino)-1-oxo-tetrahydro-thiopyran-4-ol (trans-sulfoxide)

A solution of (1S,3R,4S,5S)-3-(3-tert-butyl-benzylamino)-5-[3-fluoro-4-nitro-5-(2,2,2-trifluoro-ethoxy)-benzyl]-1-oxo-tetrahydro-thiopyran-4-ol (0.090 g, 0.163 mmol) is hydrogenated (1 atm H₂) in MeOH (6 mL) over 10% Pd—C (30 mg) for 4 h at 45° C. The catalyst is filtered off over Celite and after evaporation of the solvent the residual foam is purified by preparative HPLC (Nucleodur C₁₈, 250×19 mm, 30-100% ACN in water+0.1% TFA gradient, 30 min) to provide the title compound as a light yellow foam: TLC (CH₂Cl₂-MeOH—AcOH—H₂O 180:20:2:1) Rf=0.53; HPLC Rt_{A}=1.64 min; ESIMS [M+H]⁺=517; ¹H-NMR (400 MHz, CDCl₃): δ 7.32 (m, 3H), 7.13 (d, 1H), 6.58 (d, 2H), 6.62 (s, 2H), 4.36 (m, 2H), 3.91 (d, 1H), 3.76 (m, 3H), 3.39 (m, 1H), 3.0-3.2 (m, 3H), 2.90 (m, 1H), 2.5-2.7 (m, 3H), 2.32 (m, 1H), 1.33 (s, 9H).

### Examples 33a -33i

The compounds listed in Table 9 can be prepared by a procedure analogous to that used in example 33.

TABLE 9

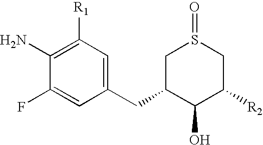

| | | | HPLC | HPLC | ESIMS |
|---|---|---|---|---|---|
| Example | R₁ | R₂ | Method | Rt [min] | [M + H]⁺ |
| 33a | 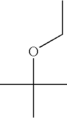 | 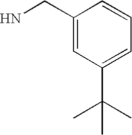 | A | 1.45 | 463 |
| 33b | 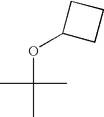 | 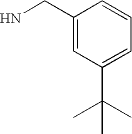 | A | 1.62 | 489 |
| 33c | 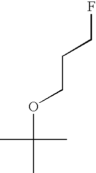 | 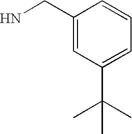 | A | 1.51 | 495 |
| 33d | 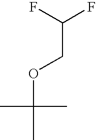 | 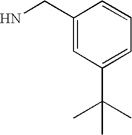 | A | 1.58 | 499 |
| 33e | 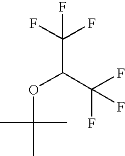 | 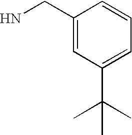 | A | 1.93 | 585 |
| 33f | 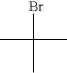 | 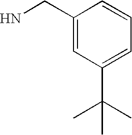 | D | 3.52 | 498/500 |
| 33g | 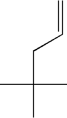 | 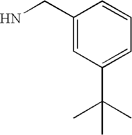 | D | 3.54 | 459 |
| 33h | 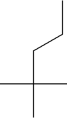 | 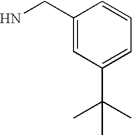 | D | 3.46 | 461 |
| 33i | 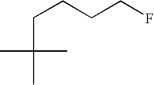 | 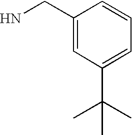 | C | 3.59 | 493 |

### Example 34

### 3S,4S,5R)-3-[4-Amino-3-fluoro-5-(2,2,2-trifluoro-ethoxy)-benzyl]-5-(3-tert-butyl-benzylamino)-1-oxo-hexahydro-1lambda*4*-thiopyran-4-ol di hydrochloride (cis-sulfoxide)

### a) {(3R,4S,5S)-5-[3-Fluoro-4-nitro-5-(2,2,2-trifluoro-ethoxy)-benzyl]-4-hydroxy-tetrahydro-thiopyran-3-yl}-carbamic acid tert-butyl ester

To a suspension of [(3R,4S,5S)-5-(3,5-difluoro-4-nitro-benzyl)-4-hydroxy-tetrahydro-thio-pyran-3-yl]-carbamic acid tert-butyl ester (example 29a) (5 g, 12.12 mmol) in THF (50 mL) is added 3,3,3-trifluoroethanol (17.6 mL, 142 mmol) and pulverized KOH (0.694 g, 12.12 mmol) and the reaction mixture is heated at reflux for 4 h. The solvent are removed under reduced pressure and the residual oil is dissolved in EtOAc, washed with brine, dried over MgSO₄, filtered and evaporated. The residue is crystallized from Et₂O-hexane to provide the title compound as light yellow crystals: TLC (hexane-EtOAc 1:1) Rf 0.27; HPLC Rt_{A}=2.28 min; ESIMS [M+H-isobutylene]⁺=429; ¹H-NMR (600 MHz, DMSO-d₆): δ 7.10 (d, 2H), 6.76 (d, 1H), 5.08 (s, 1H), 5.01 (m, 2H), 3.38 (m, 1H), 3.19 (dd, 1H), 2.92 (t, 1H), 2.55 (d, 1H), 2.48 (m, 1H), 2.37 (dt, 1H), 2.31 (d, 1H), 2.25 (dd, 1H), 1.95 (m, 1H), 1.37 (s, 9H).

### b) {(1R,3R,4S,5S)-5-[3-Fluoro-4-nitro-5-(2,2,2-trifluoro-ethoxy)-benzyl]-4-hydroxy-1-oxo-tetrahydro-thiopyran-3-yl}-carbamic acid tert-butyl ester and {(1S,3R,4S,5S)-5-[3-Fluoro-4-nitro-5-(2,2,2-trifluoro-ethoxy)-benzyl]-4-hydroxy-1-oxo-tetrahydro-thiopyran-3-yl}-carbamic acid tert-butyl ester

To a solution of {(3R,4S,5S)-5-[3-fluoro-4-nitro-5-(2,2,2-trifluoro-ethoxy)-benzyl]-4-hydroxy-tetrahydro-thiopyran-3-yl}-carbamic acid tert-butyl ester (1.774 g, 3.6 mmol) in THF-AcOH (2:1 (20 mL) is added 50% aqueous hydrogen peroxide (0.88 mL, 14.4 mmol) and the reaction mixture is stirred for 16 h at 25° C. Excess peroxide is destroyed with 10% aqueous NaS₂O₃ solution and the product is extracted with EtOAc after stirring for 0.5 h at 25° C. Combined organic layers are washed with brine, 20% aqueous K₂CO₃ solution and brine, dried over MgSO₄, filtered and evaporated. The disastereomeric sulfoxides are separated by flash-chromatography on silica gel (hexane-EtOAc-MeOH 50:50:5 to 0:20:1) to give the (1R,3R,4S,5S)-diastereomer as a colorless foam: TLC (EtOAc-MeOH 19:1) Rf 0.44; HPLC Rt_{A}=1.90 min; ESIMS [M+NH₃+H]⁺=518; ¹H-NMR (600 MHz, DMSO-d₆): δ 7.13 (s, 1H), 7.11 (d, 1H), 6.81 (d, 1H), 5.13 (d, 1H), 5.01 (m, 2H), 4.01 (m, 1H), 3.08 (m, 2H), 2.90 (d, 1H), 2.83 (d, 1H), 2.79 (m, 1H), 2.55 (m, 1H), 2.47 (dd, 1H), 2.33 (dd, 1H), 1.38 (s, 9H), and the (1S,3R,4S,5S)-diastereomer also as a colorless foam: TLC (EtOAc-MeOH 19:1) Rf 0.41; HPLC Rt_{A}=1.88 min; ESIMS [M+NH₃+H]⁺=518; ¹H-NMR (600 MHz, DMSO-d₆): δ 7.17 (s, 1H), 7.15 (d, 1H), 6.91 (d, 1H), 5.20 (d, 1H), 5.03 (m, 2H), 3.46 (m, 1H), 3.30 (d, 2H), 3.14 (m, 2H), 2.68 (dd, 1H), 2.60 (t, 1H), 2.27 (t, 1H), 1.94 (m, 1H), 1.41 (s, 9H).

### c) (1S,3R,4S,5S)-3-Amino-5-[3-fluoro-4-nitro-5-(2,2,2-trifluoro-ethoxy)-benzyl]-1-oxo-tetrahydro-thiopyran-4-ol

A solution of {(1R,3R,4S,5S)-5-[3-fluoro-4-nitro-5-(2,2,2-trifluoro-ethoxy)-benzyl]-4-hydroxy-1-oxo-tetrahydro-thiopyran-3-yl}-carbamic acid tert-butyl ester (0.92 g, 1.8 mmol) in CH₂Cl₂-TFA 6:1 (10 mL) is stirred for 2 h at 25° C. The reaction mixture is evaporated and the residue is basified with 20% aqueous K₂CO₃ solution and extracted with EtOAc. Combined organic layers are washed with brine, dried over MgSO₄, filtered and evaporated to provide the title compound as a beige amorphous solid; TLC (CH₂Cl₂-MeOH—AcOH—H₂O 180:20:2:1) Rf=0.14; HPLC Rt_{A}=1.49 min; ESIMS [M+H]⁺=401; ¹H-NMR (600 MHz, DMSO-d₆): δ 7.16 (s, 1H), 7.11 (d, 1H), 5.47 (s, 1H), 5.02 (d, 1H), 4.30 (brs, 2H), 3.28 (dd, 2H), 3.12 (dd, 1H), 3.02 (m, 2H), 2.83 (d, 1H), 2.72 (dd, 1H), 2.51 (m, 2H), 2.38 (dd, 1H).

### d) (1S,3R,4S,5S)-3-(3-tert-Butyl-benzylamino)-5-[3-fluoro-4-nitro-5-(2,2,2-trifluoro-ethoxy)-benzyl]-1-oxo-tetrahydro-thiopyran-4-ol

The title compound is prepared in an analogous manner as described for example 1h), starting from (1S,3R,4S,5S)-3-amino-5-[3-fluoro-4-nitro-5-(2,2,2-trifluoro-ethoxy)-benzyl]-1-oxo-tetrahydro-thiopyran-4-ol and 3-tert-butyl-benzaldehyde and is obtained after purification by flash-chromatography (hexane-EtOAc-MeOH 50:50:3 to 0:20:1) as a light yellow foam: TLC (AcOEt-MeOH 19:1) Rf=0.16; HPLC Rt_{A}=1.92 min; ESIMS [M+H]⁺=547; ¹H-NMR (400 MHz, CDCl₃): δ 7.32 (m, 3H), 7.14 (d, 1H), 6.83 (d, 1H), 6.79 (s, 1H), 4.45 (m, 2H), 4.23 (s, 1H), 3.92 (d, 1H), 3.75 (d, 1H), 3.54 (m, 2H), 3.42 (dt, 1H), 3.06 (m, 2H), 2.90 (m, 3H), 2.10 (m, 2H), 1.33 (s, 9H).

### e) (1S,3S,4S,5R)-3-[4-Amino-3-fluoro-5-(2,2,2-trifluoro-ethoxy)-benzyl]-5-(3-tert-butyl-benzylamino)-1-oxo-tetrahydro-thiopyran-4-ol di hydrochloride (cis-sulfoxide)

The title compound is prepared in an analogous manner as described for example 33e), starting from (1S,3R,4S,5S)-3-(3-tert-butyl-benzylamino)-5-[3-fluoro-4-nitro-5-(2,2,2-trifluoro-ethoxy)-benzyl]-1-oxo-tetrahydro-thiopyran-4-ol and the hydrochloride salt is obtained as a white powder: TLC (CH₂Cl₂-MeOH—AcOH—H₂O 180:20:2:1) Rf=0.56; HPLC Rt_{A}=1.66 min; ESIMS [M+H]⁺=517; ¹H-NMR (400 MHz, CD₃OD): δ 9.60 (s, 1H), 8.99 (s, 1H), 7.63 (s, 1H), 7.42 (d, 1H), 7.36 (m, 2H), 6.69 (s, 1H), 6.67 (d, 1H), 4.75 (m, 2H), 4.25 (m, 1H), 4.18 (m, 1H), 3.55 (m, 3H), 3.01 (d, 1H), 2.90 (t, 1H), 2.82 (dd, 1H), 2.50 (m, 1H), 2.42 (m, 2H), 1.28 (s, 9H).

### Examples 34a -34h

The compounds listed in Table 10 can be prepared by a procedure analogous to that used in example 34.

TABLE 10

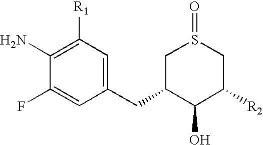

| | | | HPLC | HPLC | ESIMS |
|---|---|---|---|---|---|
| Example | R₁ | R₂ | Method | Rt [min] | [M + H]⁺ |
| 34a | 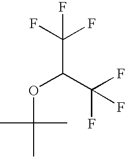 | 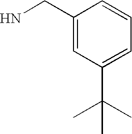 | A | 1.90 | 585 |
| 34b | 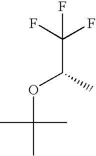 | 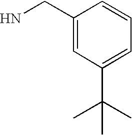 | A | 1.81 | 531 |
| 34c | 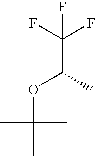 | 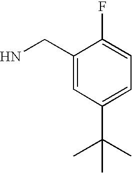 | A | 1.77 | 549 |
| 34d | 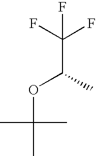 | 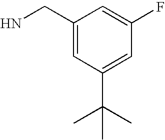 | A | 1.78 | 549 |
| 34e | 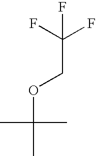 | 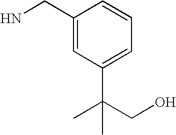 | A | 1.40 | 533 |
| 34f | 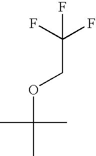 | 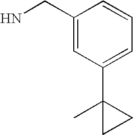 | I | 0.99 | 515 |
| 34g | 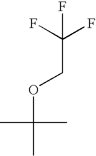 | 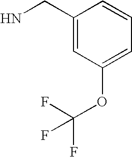 | I | 0.97 | 545 |
| 34h | 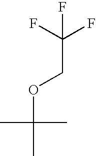 | 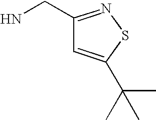 | J | 1.74 | 524 |

1. A compound of the formula 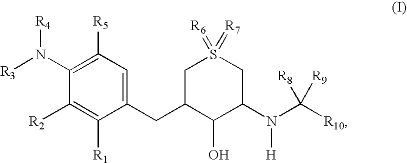
in which
R₁ is hydrogen; halogen; or (C₁₋₈)alkyl;
R₂ is hydrogen; halogen; (C₁₋₈)alkyl; halogen-(C₁₋₈)alkyl; (C₁₋₈)alkoxy; or halogen-(C₁₋₈)alkoxy;
either
R₃ is hydrogen; and
R₄ is hydrogen; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
or
R₃ is halogen-(C₁₋₈)alkyl; hydroxy-(C₁₋₈)alkyl; (C₁₋₈)alkoxy-(C₁₋₈)alkyl; formyl; (C₁₋₈)alkylcarbony; (C₃₋₈)cycloalkylcarbonyl; (C₃₋₈)cycloalkyl-(C₁₋₈)alkylcarbonyl; halogen-(C₁₋₈) alkylcarbonyl; (C₁₋₈)alkoxycarbonyl; halogen-(C₁₋₈)alkoxycarbonyl; or an aryl-(C₁₋₈)alkyl group, which aryl-(C₁₋₈)alkyl group is optionally ring-substituted by 1 to 4 substituents independenly selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₃₋₈)cycloalkyl, (C₁₋₈)alkoxy and halogen-(C₁₋₈)alkoxy; and
R₄ is hydrogen; (C₁₋₈)alkyl; (C₁₋₈)alkoxy-(C₁₋₈)alkyl; (C₁₋₈)alkylcarbonyloxy-(C₁₋₈)alkyl; formyl; (C₁₋₈)alkylcarbonyl; or (C₁₋₈)alkoxycarbonyl;
R₅ is hydrogen; halogen; (C₁₋₈)alkyl; halogen-(C₁₋₈)alkyl; (C₂₋₈)alkenyl; (C₃₋₈)cycloalkyl-(C₂₋₈)alkenyl; halogen-(C₂₋₈)alkenyl; (C₁₋₈)alkoxy; halogen-(C₁₋₈)alkoxy; (C₁₋₈)alkoxy-(C₁₋₈)alkyl; halogen-(C₁₋₈)alkoxy-(C₁₋₈)alkyl; (C₁₋₈)alkoxy-(C₁₋₈)alkoxy-(C₁₋₈)alkyl; halogen-(C₁₋₈)alkoxy-(C₁₋₈)alkoxy-(C₁₋₈)alkyl; formyl; (C₁₋₈)alkylcarbonyl; (C₃₋₈)cycloalkylcarbonyl; (C₃₋₈)cycloalkyl-(C₁₋₈)alkylcarbonyl; halogen-(C₁₋₈)alkylcarbonyl; (C₁₋₈)alkoxycarbonyl; halogen-(C₁₋₈)alkoxycarbonyl; or a (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group, which (C₃₋₈)cycloalkyl, (C₃₋₈)cycloalkyl-(C₁₋₈)alkyl, (C₃₋₈)cycloalkyl-(C₁₋₈)alkoxy, (C₃₋₈)cycloalkoxy, aryl, aryloxy, heteroaryl, heteroaryloxy, non-aromatic heterocyclyl or non-aromatic heterocyclyloxy group is optionally ring-substituted by 1 to 4 substituents independently selected from the group, consisting of halogen, (C₁₋₈)alkyl, halogen-(C₁₋₈)alkyl, (C₁₋₈)alkoxy-(C₁₋₈)alkyl, halogen-(C₁₋₈)alkoxy-(C₁₋₈)alkyl, (C₁₋₈)cycloalkyl, (C₁₋₈)alkoxy and halogen-(C₁₋₈)alkoxy;
either
R₆ is absent; and
R₇ is absent;
or
R₆ is oxo; and
R₇ is absent;
or
R₆ is oxo; and
R₇ is oxo; imino; (C₁₋₈)alkylimino; benzylimino; formylimino; or (C₁₋₈)alkylcarbonylimino;
either
R₈ is hydrogen; (C₁₋₈)alkyl; halogen-(C₁₋₈)alkyl; hydroxy-(C₁₋₈)alkyl; (C₁₋₈)alkoxy-(C₁₋₈)alkyl; or a (C₃₋₈)cycloalkyl group, which (C₃₋₈)cycloalkyl group is optionally substituted by 1 to 4 substituents independently selected from the group, consisting of halogen and (C₁₋₈) alkyl; and
R₉ is hydrogen; (C₁₋₈)alkyl; halogen-(C₁₋₈)alkyl; hydroxy-(C₁₋₈)alkyl; (C₁₋₈)alkoxy-(C₁₋₈)alkyl; or a (C₃₋₈)cycloalkyl group, which (C₃₋₈)cycloalkyl group is optionally substituted by 1 to 4 substituents independently selected from the group, consisting of halogen and (C₁₋₈)alkyl;
or
R₈ and R₉, taken together, complete, together with the carbon atom, to which they are attached, a (C₃₋₈)cycloalkylidene moiety, in which (C₃₋₈)cycloalkylidene moiety 1 of its —CH₂— ring members can be replaced with —O—; and
R₁₀ is an aryl or heteroaryl group, which aryl or heteroaryl group is optionally mono-, di-, tri- or tetra-substituted by substituents independently selected from the group, consisting of halogen, hydroxy, (C₁₋₈)alkyl, halogen-(C₁₋₈)alkyl, hydroxy-(C₁₋₈)alkyl, hydroxy-(C₁₋₈)alkyl substituted by halogen, (C₁₋₈)alkoxy-(C₁₋₈)alkyl, halogen-(C₁₋₈)alkoxy-(C₁₋₈)alkyl, cyano-(C₁₋₈)alkyl, (C₁₋₈)alkoxy, halogen-(C₁₋₈)alkoxy, a heteroaryl group, which heteroaryl group is optionally substituted by 1 to 4 substituents independently selected from the group, consisting of halogen, (C₁₋₈)alkyl and halogen-(C₁₋₈)alkyl, and a (C₃₋₈)cycloalkyl group, in which (C₃₋₈)cycloalkyl group 1 of its —CH₂— ring members can be replaced with —O—, and which (C₃₋₈)cycloalkyl group, in which 1 of its —CH₂— ring members is optionally replaced with —O—, is optionally substituted by 1 to 4 substituents independently selected from the group, consisting of halogen, (C₁₋₈)alkyl and halogen-(C₁₋₈)alkyl, in free form or in salt form.

2. A process for the preparation of a compound as defined in claim 1 of the formula I, in free form or in salt form, comprising the steps of
a) for the preparation of a compound of the formula I, in free form or in salt form, in which R₃ is hydrogen and R₄ is hydrogen, treatment of a compound of the formula 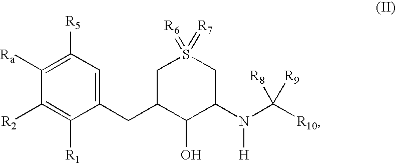
in which Rₐ is azido or nitro and all of the other variables are as defined for the formula I, in free form or in salt form, with a reducing agent, in order to convert Rₐ into amino, or
b) for the preparation of a compound of the formula I, in free form or in salt form, in which R₈ is hydrogen, treatment of a compound of the formula 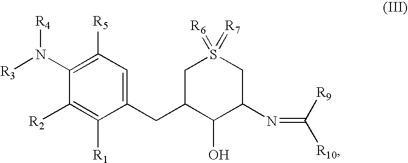
in which all of the variables are as defined for the formula I, in free form or in salt form, with a reducing agent, in order to convert the moiety —N═C(R₉)R₁₀ into the moiety —N(H)—C(H)(R₉)R₁₀.
in each case optionally followed by reduction, oxidation or other functionalisation of the resulting compound and/or by cleavage of any protecting group(s) optionally present,
and of recovering the so obtainable compound of the formula I in free form or in salt form.

3-4. (canceled)

5. A pharmaceutical composition, comprising:
a the compound as defined in claim 1, in free form or in pharmaceutically acceptable salt form, as active ingredient and
a pharmaceutical carrier or diluent.

6-7. (canceled)

8. A method for the treatment of neurological or vascular disorders related to beta-amyloid generation and/or aggregation in a subject in need of such treatment, comprising:
administering to such subject a therapeutically effective amount of the compound as defined in claim 1, in free form or in pharmaceutically acceptable salt form.

9. A combination, comprising:
a therapeutically effective amount of the compound as defined in claim 1, in free form or in pharmaceutically acceptable salt form, and
a second drug substance, for simultaneous or sequential administration.
